(19) United States
(12) Patent Application Publication
(10) Pub. No.: US 2026/0100268 A1
(43) Pub. Date: Apr. 9, 2026

(54) SYSTEMS AND METHODS FOR EMS ENCOUNTER RECORDS

(71) Applicant: ZOLL Medical Corporation, Chelmsford, MA (US)

(72) Inventors: Corissa J. Bowman, Golden, CO (US); Stephen A. Frye, Broomfield, CO (US); Keenan S. Early, Denver, CO (US); Frederick W. Forester, Encinitas, CA (US); Eric H. Strand, Marshfield, MA (US); Adam C. Mihlfried, Pittsburgh, PA (US); Gordon P. Nall, San Diego, CA (US); Jared K. Williams, Broomfield, CO (US); Burton Daniel Nayman, Raleigh, NC (US); Peter G. Goutmann, Gibsonia, PA (US)

(73) Assignee: ZOLL Medical Corporation, Chelmsford, MA (US)

(21) Appl. No.: 18/864,115

(22) PCT Filed: May 9, 2023
(86) PCT No.: PCT/US2023/021430

§ 371 Date: Nov. 8, 2024

Related U.S. Application Data
(60) Provisional application No. 63/339,833, filed on May 9, 2022.

(51) Int. Cl. G16H 40/20; G16H 10/65

(52) U.S. Cl. CPC G16H 40/20; G16H 10/65

(57) ABSTRACT

A smartphone is provided for documenting an emergency medical services (EMS) call within an electronic patient care record (ePCR). The smartphone includes a memory storing an ePCR including data fields; a touchscreen; and a processor coupled to the memory and the touchscreen. The processor is configured to provide a first data entry screen through the touchscreen, the first data entry screen including user interface controls configured to receive ePCR data field entries through touchscreen gestures, receive the ePCR data field entries through the user interface controls, store each ePCR data field entry in a respective data field of the data fields, identify at least one second data entry screen based on a data field entry of the ePCR data field entries, and provide the second data entry screen through the touchscreen.

### CROSS REFERENCE TO RELATED APPLICATIONS

This application claims priority under 35 U.S.C. § 119(e) to U.S. Provisional Application Ser. No. 63/339,833, titled “SYSTEMS AND METHODS FOR EMS ENCOUNTER RECORDS,” filed May 9, 2022, which is hereby incorporated herein by reference in its entirety.

### BACKGROUND

The present disclosure is directed to systems and methods for emergency medical services (EMS) encounter recording. These systems and methods are crafted to provide efficient and accurate contemporaneous records within the constraints of an EMS environment.

EMS agencies create and use an electronic patient care record (ePCR) for each patient encounter. Even if a particular patient has been treated during multiple encounters with one or more EMS agencies, there will be a newly generated and separate ePCR for each encounter with each agency. This stands in contrast to a patient medical record generated by a physician where the record follows the patient and includes information about multiple encounters with the physician for that same patient. The ePCR contains a complete and time-stamped record of medical observations, interventions and treatments, and transport for the patient during a patient encounter. Due to the intricacies of medical care along with governmental reporting guidelines, the ePCR is typically a complex and lengthy document.

Software applications exist that interact with EMS personnel to complete ePCRs. These software applications include user interface screens with controls to receive input from EMS personnel regarding the patient encounter. This input specifies values of data fields that document the complete encounter record described above.

### SUMMARY

In an example, a smartphone device for documenting an emergency medical services (EMS) call within an electronic patient care record (ePCR) is provided. The smartphone device includes a memory storing an ePCR including a plurality of data fields; a touchscreen display; and at least one processor coupled to the memory and the touchscreen display. The at least one processor is and configured to provide a first data entry screen of a plurality of data entry screens through the touchscreen display, the plurality of data entry screens including a plurality of user interface controls configured to receive a plurality of ePCR data field entries through touchscreen gestures, receive the plurality of ePCR data field entries from an EMS caregiver through the plurality of user interface controls, store each ePCR data field entry of the plurality of ePCR data field entries in a respective data field of the plurality of data fields, identify at least one second data entry screen of the plurality of data entry screens based on at least one data field entry of the plurality of ePCR data field entries, and provide the at least one second data entry screen through the touchscreen display.

Examples of the smartphone device can incorporate one or more of the following features. In the smartphone device, the first data entry screen may be a patient demographics importation screen and the at least one second data entry screen may include a patient demographics editing screen. The memory may store shift information specifying at least one base location of the EMS caregiver; and the at least one processor may be configured to receive gesture input through the touchscreen display requesting to access trip information, and provide, in response to the gesture input, a trip information screen through the touchscreen display, the trip information screen including a textual representation of at least a portion of the shift information. The shift information may further specify a vehicle, a staffing level, and a shift number; and the trip information screen may include textual representations of the vehicle, the staffing level, and the shift number. The smartphone device may include a network interface. The at least one processor may be configured to receive dispatch information from a computer aided dispatch (CAD) system through the network interface. The trip information screen may include a textual representation of at least a portion of the dispatch information. The dispatch information may include one or more of dispatch priority, type of service, and an indication of whether the EMS call was scheduled; and the trip information screen may include textual representations of the dispatch priority, the type of service, and the indication of whether the EMS call was scheduled.

In the smartphone device, the at least one processor may be configured to receive gesture input via the touchscreen display specifying a change to the dispatch information; and communicate the change to the dispatch information to the CAD system via the network interface. The at least one processor may be configured to receive billing information via the network interface; provide a billing screen through the touchscreen display, the billing screen including a textual representation of at least a portion of the billing information; receive gesture input via the touchscreen display specifying a change to the at least the portion of the billing information; and communicate the change to the at least the portion of the billing information to a billing system via the network interface. The at least one processor may be configured to communicate, via the network interface, ePCR data stored in one or more data fields of the plurality of data fields to a data analytics system; and receive, from the data analytics system via the network interface, information based on the ePCR data. The PCR data may be demographic data; and the information may supplement the demographic data. The ePCR data may be demographic data; and the information may correct the demographic data. The ePCR data may include one or more of insurance data or demographic data; and the information includes one or more of insurance verification or insurance identification information. The ePCR data may specify one or more of an insurance carrier, group name, group number, policy identifier, policy holder, and policy priority. The ePCR data may include one or more of insurance data or demographic data; and the information may specify previous claims data. The network interface may include one or more of a cellular interface, a WiFi interface, or a proximal interface. The smartphone may include a global positioning system chipset. The the dispatch information may include destination information. The at least one processor may be configured to determine a route based on the destination information.

In the smartphone device, the at least one processor may be configured to provide a times entry screen including a plurality of time and date controls, the plurality of time and date controls including at least one current timestamp control; receive gesture input through the touchscreen display selecting the at least one current timestamp control; and store, in response to reception of the gesture input, a current timestamp in at least one data field of the plurality of data fields associated with the at least one current timestamp control. The at least one processor may be configured to provide a chart selection screen including an add chart control; receive gesture input through the touchscreen display selecting the add chart control; and allocate, in response to reception of the gesture input, space for an additional ePCR in the memory.

In the smartphone device, the at least one processor may be configured to provide a quick action (QA) screen associated with a particular patient condition through the touchscreen display including a plurality of QA controls associated with treatments for the particular patient condition; receive gesture input through the touchscreen display selecting a QA control of the plurality of QA controls; and store, in response to reception of the gesture input, ePCR data in one or more data fields of the plurality of data fields, the ePCR data specifying a current timestamp and performance of an action associated with the QA control. The the QA screen may be a cardiac condition QA screen. The QA control may be a cardiac condition QA control. The the action may be administration of the cardiac treatment to a patient. The QA screen may be a trauma QA screen. The QA control may be a trauma QA control. The action may be administration of the trauma treatment to a patient. The QA screen may be a respiratory distress QA screen. The QA control may be a respiratory distress QA control. The action may be administration of the respiratory distress treatment to a patient. The QA screen may be a sepsis QA screen. The QA control may be a sepsis QA control. The action may be administration of the sepsis treatment to a patient.

In the smartphone device, the at least one processor may be configured to toggle a timer associated with the QA control in response to reception of the gesture input. The at least one processor may be configured to provide at least one highlight to the QA control in response to expiration of the timer. The at least one highlight may include a red header. The expiration may be configurable. The action may be part of a medical response protocol and the expiration may be set based on the medical response protocol.

The smartphone device may further include a camera. In the smartphone device, the at least one processor may be configured to provide an attachments screen including a scan control; receive gesture input through the touchscreen display selecting the scan control; control the device to acquire one or more images through the scan control; and store, in the memory, the one or more images as attachments to the ePCR. The scan control may include a patient identification control. The one or more images may include an image of a fiducial of patient identification documentation. The at least one processor may be configured to extract demographic data from the fiducial. The at least one processor may be configured to provide a demographics importation screen including an item control; receive gesture input through the touchscreen display selecting the item control; and toggle, in response to reception of the gesture input, selection of the item control. The demographics importation screen may include a save control. The at least one processor may be configured to receive gesture input through the touchscreen display selecting the save control, and store, in response to reception of the gesture input, demographic data associated with the item control in one or more data fields of the plurality of data fields. The EMS caregiver may be a member of a second EMS team providing patient care subsequent to a first EMS team. The ePCR may be a second ePCR. The at least one processor may be configured to receive data specifying that the first EMS team was assigned to the EMS call, the data including a driver's license number; receive ePCR data generated by the first EMS team in a first ePCR prior to arrival of the second EMS team; and provide the ePCR data from the first ePCR at the smartphone device. The ePCR data may include one or more of insurance information, EMS treatment information, medication information, or allergy information.

In the smartphone device, to receive the data specifying that the first EMS team was assigned to the EMS call may include to acquire an image of a fiducial. The at least one processor may be configured to receive user input authorizing receipt of the ePCR data prior to receipt of the ePCR data. The smartphone device may be configured to provide the first ePCR data within at least one user interface control configured to indicate that the ePCR data was generated prior to arrival of the second EMS team. The smartphone device may be configured to receive at least one new ePCR data field entry; and provide, within a common screen, the at least one user interface control and at least one other user interface control displaying the at least one new ePCR data field entry.

In the smartphone device, the at least one processor may be configured to provide a medical history screen including a plurality of history item controls; receive gesture input through the touchscreen display to toggle selection of a history item control of the plurality of history item controls; store, if the history item control is toggled on, ePCR data in one or more data fields of the plurality of data fields, the ePCR data specifying a medical condition associated with the history item control; and delete, if the history item control is toggled off, ePCR data from one or more data fields of the plurality of data fields, the ePCR data specifying the medical condition associated with the history item control. The at least one processor may be configured to provide a share screen including a submit control; receive gesture input through the touchscreen display to select the submit control; and upload, in response to reception of the gesture input, the ePCR to a remote server. The at least one processor may be configured to receive, from the remote server, a uniform resource locator (URL) endpoint through which the ePCR is accessible; encode the URL in a fiducial; and provide the fiducial through the touchscreen display. In the smartphone device, to upload may include to communicate the ePCR using HL7.

In the smartphone device, the EMS caregiver may be a member of a second EMS team providing patient care subsequent to a first EMS team. The ePCR may be a second ePCR. The at least one processor may be configured to receive data specifying that the first EMS team was assigned to the EMS call; receive ePCR data generated by the first EMS team prior to arrival of the second EMS team; and provide the ePCR data from the first ePCR in a visual representation of the second ePCR. The ePCR data may include one or more of insurance information, EMS treatment information, medication information, or allergy information. The data specifying that the first EMS team was assigned to the EMS call may include a reference code of a patient. The reference code may be a driver's license number. In the smartphone device, to receive the data specifying that the first EMS team was assigned to the EMS call may include to acquire an image of a fiducial. The at least one processor may be configured to receive user input authorizing receipt of the ePCR data prior to receipt of the ePCR data. The smartphone device may be configured to provide the ePCR data within at least one user interface control configured to indicate that the ePCR data was generated prior to arrival of the second EMS team. The smartphone device may be configured to receive at least one new ePCR data field entry; and provide, within a common screen, the at least one user interface control and at least one other user interface control displaying the at least one new ePCR data field entry. In the smartphone device, the received ePCR data may be from a first ePCR distinct from the second ePCR and associated with the first EMS team; and the new ePCR data field entry is associated with the second ePCR distinct from the first ePCR. The second ePCR may be associated with the second EMS team and the first ePCR may be associated with the first EMS team. Each of the first ePCR and the second ePCR may be specific to a combination of patient and EMS team. The smartphone device may be configured to receive additional ePCR data generated after the patient is transferred to a healthcare provider other than the first EMS team and the second EMS team; and provide, within a chronology screen, ePCR data generated by the first EMS team, the second EMS team, and the healthcare provider, the ePCR data including an outcome of the EMS call.

In the smartphone device, the at least one processor may be configured to receive a swipe gesture through a control of the plurality of user interface controls; and alter the control to include one or more of a delete control and a more control. The touchscreen display has a diagonal length of less than 19.5 cm. The smartphone device may further include a network interface. In the smartphone device, the at least one processor may be configured to receive, via the network interface, data field values from a plurality of mobile devices including two or more data field values for a same data field in the ePCR, and execute a charting data synchronization service to identify an adjudication scheme associated with the same data field, and apply the adjudication scheme to determine an adjudicated value from the two or more data field values for the same data field in the ePCR. The data field values may be received during a first time period in which the network interface lacked connectivity to a remote server. The charting data synchronization service may be configured to store, in a local data store, the data field values during the first time period, monitor network connectivity to the remote server, identify a resumption or initiation of the network connectivity to the remote server, and send the data field values to the remote server during a second time period in which the network interface has connectivity to the remote server. Two or more data field values for the same data field in the ePCR may include data field values received at two or more mobile devices during the first time period in which the two or more mobile devices lacked network connectivity to the remote server. The two or more data field values for the same data field in the ePCR may include a first data field value received at a first mobile device during the first time period and a second data field value received at a second mobile device during the second time period. The adjudication scheme may be configured to identify an adjudicated value from the first data field value and the second data field value. The charting data synchronization service may be configured to maintain a queue configured to store data field values, maintain a list of subscribers to the data field values including an instance of the charting data synchronization service hosted on the remote server, maintain a list of publishers of the data field values, the list of publishers including a charting application, read the data field values stored in the memory, enqueue the data field values to the queue during the first time period, dequeue the data field values from the queue during the second time period, and send the data field values to the list of subscribers during the second time period.

In the smartphone device, the at least one processor may be configured to provide a medications screen including one or more medication control groups associated with one or more medications; receive gesture input through the touchscreen display specifying medication information regarding the one or more medications; and store, in response to reception of the gesture input, ePCR data in one or more data fields of the plurality of data fields, the ePCR data specifying the medication information. The one or more medication control groups may include one or more name controls and the medication information may include one or more names of the one or more medications. The one or more medication control groups may include one or more dose controls and the medication information may include one or more doses of the one or more medications. The one or more medication control groups may include one or more dose controls and the medication information may include one or more doses of the one or more medications. The one or more medication control groups may include one or more dose unit controls and the medication information may include one or more dose units of the one or more medications. The one or more medication control groups may include one or more route controls and the medication information may include one or more routes of the one or more medications. The one or more medication control groups may include one or more frequency controls and the medication information may include one or more frequencies of the one or more medications.

In the smartphone device, the at least one processor may be configured to provide a chronology screen through the touchscreen display including a plurality of time entry control groups associated with a plurality of timeline entries, each of the plurality of time entry control groups including at least one time entry control and at least one source control; and provide, via the at least one source control, an indication of a source of a corresponding timeline entry of the plurality of timeline entries. The indication may be of a medical device. The medical device may include one or more of a patient monitor, a public access automated external defibrillator, a professional defibrillator/patient monitor, a ventilator, or an automated compression device. The indication may be of an EMS platform service. The EMS platform service may include one or more of a computer aided dispatch system, a navigation system, a billing system, a charting system, a case data store, a data analytics system, a hospital information system, or a medical data repository. The least one processor may be configured to provide, via the at least one time entry control, a visual representation of a timeline entry of the plurality of timeline entries. The visual representation may be of one or more of a call time, a dispatch time, a response time, an on-scene time, a time at which the EMS caregiver reached a patient, a time at which the patient's vitals were measured, a time at which medication was administered to the patient, a time at which patient transport to a hospital initiated, a time at which the patient was admitted to the hospital, a time at least the patient was discharged from the hospital.

In the smartphone device, each of the plurality of time entry control groups may include at least one owner control. The least one processor may be configured to provide, via the at least one owner control, an indication of the owner of a corresponding timeline entry of the plurality of timeline entries. The indication of the owner may be an indication of one or more of a basic life support (BLS) team, an advanced life support (ALS) team, or a hospital department. The plurality of time entry control groups may include a first time entry control group, a second time entry control group, and a third time entry control group. The first time entry control group may include a first owner control that indicates a first corresponding timeline entry of the plurality of timeline entries is owned by the BLS team. The second time entry control group may include a second owner control that indicates a second corresponding timeline entry of the plurality of timeline entries is owned by the ALS team. The third time entry control group may include a third owner control that indicates a third corresponding timeline entry of the plurality of timeline entries is owned by the hospital department.

In another example, an emergency medical services (EMS) charting system for documenting an EMS call is provided. The system includes a remote server including at least one processor including a charting data synchronization service and a memory storing an electronic patient care record (ePCR) for the EMS call, a plurality of mobile devices associated with an EMS call environment, each mobile device including a network interface configured to communicatively couple the mobile device to the remote server, and a memory storing data field values for one or more data fields of the ePCR, wherein the at least one processor is configured to receive, via the network interface, data field values from the plurality of mobile devices including two or more data field values for a same data field in the ePCR, and execute the charting data synchronization service to: identify an adjudication scheme associated with the same data field, and apply the adjudication scheme to determine an adjudicated value from the two or more data field values for the same data field in the ePCR.

Examples of the EMS charting system can incorporate one or more of the following features. In the EMS charting system, the remote server may be one or more of a cloud server or a mobile server. The data field values may be received by at least one mobile device during a first time period in which the at least one mobile device lacked connectivity to the remote server; and the charting data synchronization service may be configured to store, in a local data store, the data field values during the first time period, monitor network connectivity to the remote server for the at least one mobile device, identify a resumption or initiation of the network connectivity to the remote server for the at least one mobile device, and send the data field values to the remote server during a second time period in which the at least one mobile device has network connectivity to the remote server. The two or more data field values for the same data field in the ePCR may include data field values may be received at two or more mobile devices during the first time period in which the two or more mobile devices lacked network connectivity to the remote server. The two or more data field values for the same data field in the ePCR may include a first data field value received at a first mobile device during the first time period and a second data field value received at a second mobile device during the second time period. The adjudication scheme may be configured to identify an adjudicated value from the first data field value and the second data field value. In the EMS charting system, each mobile device may include a charting application configured to provide a user interface to capture the data field values from a user, and store the data field values in the memory; and the charting data synchronization service is configured to maintain a queue configured to store data field values, maintain a list of subscribers to the data field values including an instance of the charting data synchronization service hosted on the remote server, maintain a list of publishers of the data field values, the list of publishers including the charting application, read the data field values stored in the memory, enqueue the data field values to the queue during the first time period, dequeue the data field values from the queue during the second time period, and send the data field values to the list of subscribers during the second time period.

In the EMS charting system, the adjudication scheme may be configured to apply a timestamp adjustment to data field values originating from different mobile devices. The adjudication scheme may be a first in wins method configured to identify a data field value associated with an earliest timestamp relative to timestamps associated with other data field values; and to apply the adjudication scheme may include to identify the adjudicated value as a data field value of the two or more data field values associated with the earliest timestamp. The adjudication scheme may be a last in wins method configured to identify a data field value associated with the latest timestamp relative to timestamps associated with other data field values; and to apply the adjudication scheme may include to identify the adjudicated value as a data field value of the two or more data field values associated with the latest timestamp. The adjudication scheme may be a majority vote wins method configured to identify a data field value of a majority of received data field values; and to apply the adjudication scheme may include to identify the adjudicated value as a data field value of a majority of the two or more data field values.

In the EMS charting system, the adjudication scheme may be an authoritative source wins method; and to apply the adjudication scheme may include to identify at least one role associated with the two or more data field values, identify an authoritative role from the at least one role, and identify the adjudicated value as the data field value that originated from the authoritative role. In the EMS charting system, the first data field value of the two or more data field values may originate from an emergency medical responder; the second data field value of the two or more data field values may originate from an emergency medical technician; the third data field value of the two or more data field values may originate from an advanced emergency medical technician; the fourth data field value of the two or more data field values may originated from a paramedic; and to apply the adjudication scheme may include to identify the fourth data field value as the adjudicated value.

In the EMS charting system, to apply the adjudication scheme may include to apply a machine learning model to features of the two or more data field values. To apply the adjudication scheme may include to generate a confidence metric. In the EMS charting system, the charting data synchronization service may be configured to determine if the confidence metric transgresses a threshold; and request user verification where the confidence metric does not transgress the threshold. The charting data synchronization service may be configured to store the adjudicated value within the same data field.

In the EMS charting system, the plurality of mobile devices may include at least one smartphone device including a touchscreen display. The one or more data fields of the ePCR may include a plurality of data fields and the at least one smartphone device may be configured to provide a first data entry screen of a plurality of data entry screens through the touchscreen display, the plurality of data entry screens including a plurality of user interface controls configured to receive a plurality of ePCR data field entries through touchscreen gestures; receive the plurality of ePCR data field entries from an EMS caregiver through the plurality of user interface controls; store each ePCR data field entry of the plurality of ePCR data field entries in a respective data field of the plurality of data fields; identify at least one second data entry screen of the plurality of data entry screens based on at least one data field entry of the plurality of ePCR data field entries; and provide the at least one second data entry screen through the touchscreen display. The one or more data fields of the ePCR may be configured to store one or more of transport information, medical information, and demographic information. In the EMS charting system, one or more of the remote server or the at least one smartphone device may be configured to receive data form a medical device. The network interface may include one or more of a cellular interface, a WiFi interface, or a proximal interface. The at least one smartphone device may be configured to provide a quick action (QA) screen through the touchscreen display including a plurality of QA controls; receive gesture input through the touchscreen display selecting a QA control of the plurality of QA controls; and store, in response to reception of the gesture input, ePCR data in one or more data fields of the plurality of data fields, the ePCR data specifying a current timestamp and performance of an action associated with the QA control. The QA screen may be a cardiac arrest QA screen. The QA control may be a cardioversion QA control. The action may be administration of cardioversion to a patient. The QA screen may be a trauma QA screen. The QA control may be a c-spine stabilize QA control. The action may be administration of c-spine stabilization to a patient. The at least one processor may be configured to toggle a timer associated with the QA control in response to reception of the gesture input. The at least one processor may be configured to provide at least one highlight to the QA control in response to expiration of the timer. The at least one highlight may include a red header. The expiration may be configurable. The action is part of a treatment protocol, and the expiration is set based on the treatment protocol.

In another example, an emergency medical services (EMS) charting system for documenting an EMS call is provided. The system includes a first mobile device configured to receive user input specifying first data to be stored in a first electronic patient care record (ePCR), store the first data in the first ePCR in response to receipt of the user input, and selectively forward the first data stored in the first ePCR to other mobile devices dispatched to the EMS call; and a second mobile device configured to receive other user input specifying second data to be stored in a second electronic patient care record (ePCR), store the second data in the second ePCR in response to receipt of the other user input, selectively forward the second data stored in the second ePCR to the other mobile devices dispatched to the EMS call, receive the first data from the first mobile device, display, via a user interface, the first data in a common screen with the second data, and indicate that the first data was generated prior to arrival of the second mobile device at the EMS call.

Examples of the EMS charting system can incorporate one or more of the following features. In the EMS charting system, the first mobile device and the second mobile device may each be one of a smartphone, a tablet computing device, a watch computing device or other wearable computing device, a heads-up glasses display, an augmented reality display device, a virtual reality display device, or a medical device display. The second mobile device may be associated with a member of a second EMS team and configured to receive data specifying that a first EMS team is assigned to the EMS call, the data including a reference code of a patient; and receive the first data generated by the first EMS team prior to arrival of the second EMS team. The first data may include one or more of insurance information, EMS treatment information, medication information, or allergy information. In the EMS charting system, to receive the data specifying that the first EMS team is assigned to the EMS call may include to acquire an image of a fiducial. The second mobile device may be configured to receive user input authorizing receipt of the first data prior to receipt of the first data. The reference code is a driver's license number. In the EMS charting system, the first mobile device may be configured to receive additional data generated after a patient is transferred to a healthcare provider other than the first EMS team and the second EMS team; and display, within a chronology screen, additional data generated by the first EMS team, the second EMS team, and the healthcare provider, the additional data including an outcome of the EMS call.

In the EMS charting system, each of the first ePCR and the second ePCR may be specific to a combination of patient and EMS team. The first mobile device may be configured to display a medications screen including one or more medication control groups associated with one or more medications; receive gesture input specifying medication information regarding the one or more medications; and store, in response to reception of the gesture input, data specifying the medication information. The one or more medication control groups may include one or more name controls, and the medication information may include one or more names of the one or more medications. The one or more medication control groups may include one or more dose controls, and the medication information may include one or more doses of the one or more medications. The one or more medication control groups may include one or more dose controls, and the medication information may include one or more doses of the one or more medications. The one or more medication control groups may include one or more dose unit controls, and the medication information may include one or more dose units of the one or more medications. The one or more medication control groups may include one or more route controls, and the medication information may include one or more routes of the one or more medications. The one or more medication control groups may include one or more frequency controls, and the medication information may include one or more frequencies of the one or more medications.

In the EMS charting system, the first mobile device may be configured to display a chronology screen including a plurality of time entry control groups associated with a plurality of timeline entries, each of the plurality of time entry control groups including at least one time entry control and at least one source control; and display, via the at least one source control, an indication of a source of a corresponding timeline entry of the plurality of timeline entries. The indication may be of a medical device. The medical device may include one or more of a patient monitor, defibrillator, ventilator, or automated compression device. The indication may be of an EMS platform service. The EMS platform service may include one or more of a computer aided dispatch system, a navigation system, a billing system, a charting system, a case data store, a data analytics system, a hospital information system, or a medical data repository. The at least one processor may be configured to provide, via the at least one time entry control, a visual representation of a timeline entry of the plurality of timeline entries. The visual representation may be of one or more of a call time, a dispatch time, a response time, an on-scene time, a time at which the EMS caregiver reached a patient, a time at which the patient's vitals were measured, a time at which medication was administered to the patient, a time at which patient transport to a hospital initiated, a time at which the patient was admitted to the hospital, or a time at least the patient was discharged from the hospital.

In the EMS charting system, each of the plurality of time entry control groups may include at least one owner control; and the first mobile device may be configured to display, via the at least one owner control, an indication of the owner of a corresponding timeline entry of the plurality of timeline entries. The indication of the owner may be an indication of one or more of a basic life support (BLS) team, an advanced life support (ALS) team, or a hospital department. The plurality of time entry control groups may include a first time entry control group, a second time entry control group, and a third time entry control group; the first time entry control group may include a first owner control that indicates a first corresponding timeline entry of the plurality of timeline entries is owned by the BLS team; the second time entry control group may include a second owner control that indicates a second corresponding timeline entry of the plurality of timeline entries is owned by the ALS team; and the third time entry control group may include a third owner control that indicates a third corresponding timeline entry of the plurality of timeline entries is owned by the hospital department.

In the EMS charting system, the first mobile device may be configured to communicate additional data stored in the first ePCR to a data analytics system; and receive further information regarding the additional data from the data analytics system. The additional data may be demographic data; and the further information may supplement the demographic data. The additional data may be demographic data; and the further information may correct the demographic data. The additional data may be insurance data; and the further information may indicate the insurance data is verified. The additional data may specify one or more of an insurance carrier, group name, group number, policy identifier, policy holder, and policy priority. The additional data may be insurance data; and the further information may specify previous claims data.

In the EMS charting system, the first mobile device may be configured to communicate additional data stored in the first ePCR to a computer aid dispatch (CAD) system; and receive further information regarding the additional data from the CAD system. The further information may include dispatch information. The dispatch information may include a patient reference code. The patient reference code may a driver's license number. The first mobile device may be configured to scan a fiducial to identify the driver's license number. The first mobile device may be configured to communicate additional data stored in the first ePCR to a charting system; and receive further information regarding the additional data from the charting system. The further information may include records of previous encounters with a patient. The records of previous encounters may include physiologic data of the patient. The physiologic data may include electrocardiogram data.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples disclosed herein and are incorporated in and constitute a part of this specification. However, the figures are not intended to limit the scope of the disclosure. The figures, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1A illustrates front views of a login screen, a shift startup screen, and a chart selection screen provided by a mobile EMS charting application in accordance with at least one example disclosed herein.

FIG. 1B illustrates front views of a new chart screen and a quick action (QA) screen provided by a mobile EMS charting application in accordance with at least one example disclosed herein.

FIG. 1C illustrates a front view of the new chart screen provided by a mobile EMS charting application in accordance with at least one example disclosed herein.

FIG. 1D illustrates front views of a trip information screen and a times entry screen provided by a mobile EMS charting application in accordance with at least one example disclosed herein.

FIG. 1E illustrates front views of an attachments screen, a scan screen, a driver's license importation screen, and a patient demographics screen provided by a mobile EMS charting application in accordance with at least one example disclosed herein.

FIG. 1F illustrates front views of a medications importation screen, a medical history screen, and a share screen provided by a mobile EMS charting application in accordance with at least one example disclosed herein.

FIG. 2A is a block diagram illustrating one example of an EMS charting system in accordance with at least one example disclosed herein.

FIG. 2B is a block diagram illustrating another example of an EMS charting system in accordance with at least one example disclosed herein.

FIG. 2C is a block diagram of a synchronization infrastructure in accordance with at least one example disclosed herein.

FIG. 3 is a flow diagram illustrating a portion of a process of documenting an EMS call within an ePCR in accordance with at least one example disclosed herein.

FIG. 4 illustrates a front view of a chart selection screen and portions thereof in accordance with at least one example disclosed herein.

FIG. 5 is a flow diagram illustrating another portion of a process of documenting an EMS call within an ePCR in accordance with at least one example disclosed herein.

FIGS. 6A and 6B illustrate front views of a new chart selection screen in accordance with at least one example disclosed herein.

FIG. 7 is a flow diagram illustrating another portion of a process of documenting an EMS call within an ePCR in accordance with at least one example disclosed herein.

FIG. 8 illustrates a front view of a chart edit screen in accordance with at least one example disclosed herein.

FIG. 9 is a flow diagram illustrating another portion of a process of documenting an EMS call within an ePCR in accordance with at least one example disclosed herein.

FIG. 10 illustrates a front view of a timeline edit screen in accordance with at least one example disclosed herein.

FIG. 11 is a flow diagram illustrating another portion of a process of documenting an EMS call within an ePCR in accordance with at least one example disclosed herein.

FIG. 12 illustrates a front view of a patient information screen in accordance with at least one example disclosed herein.

FIG. 13 is a flow diagram illustrating another portion of a process of documenting an EMS call within an ePCR in accordance with at least one example disclosed herein.

FIG. 14 illustrates a front view of a medical history screen in accordance with at least one example disclosed herein.

FIG. 15 is a flow diagram illustrating another portion of a process of documenting an EMS call within an ePCR in accordance with at least one example disclosed herein.

FIG. 16 illustrates a front view of an allergies screen in accordance with at least one example disclosed herein.

FIG. 17 is a flow diagram illustrating another portion of a process of documenting an EMS call within an ePCR in accordance with at least one example disclosed herein.

FIG. 18 illustrates a front view of a times entry screen in accordance with at least one example disclosed herein.

FIG. 19 is a flow diagram illustrating another portion of a process of documenting an EMS call within an ePCR in accordance with at least one example disclosed herein.

FIG. 20 illustrates a front view of an actions navigation screen in accordance with at least one example disclosed herein.

FIG. 21 is a flow diagram illustrating another portion of a process of documenting an EMS call within an ePCR in accordance with at least one example disclosed herein.

FIG. 22 illustrates a front view of a cardiac arrest QA screen and portions thereof in accordance with at least one example disclosed herein.

FIG. 23 is a flow diagram illustrating another portion of a process of documenting an EMS call within an ePCR in accordance with at least one example disclosed herein.

FIG. 24 illustrates a front view of a trauma QA screen in accordance with at least one example disclosed herein.

FIG. 25 is a flow diagram illustrating another portion of a process of documenting an EMS call within an ePCR in accordance with at least one example disclosed herein.

FIG. 26 illustrates a front view of an attachments screen in accordance with at least one example disclosed herein.

FIG. 27 is a flow diagram illustrating another portion of a process of documenting an EMS call within an ePCR in accordance with at least one example disclosed herein.

FIG. 28 illustrates a front view of a scan screen in accordance with at least one example disclosed herein.

FIG. 29 illustrates a front view of a demographics importation screen in accordance with at least one example disclosed herein.

FIG. 30 is a flow diagram illustrating another portion of a process of documenting an EMS call within an ePCR in accordance with at least one example disclosed herein.

FIG. 31 illustrates a front view of a signatures screen in accordance with at least one example disclosed herein.

FIG. 32 is a flow diagram illustrating another portion of a process of documenting an EMS call within an ePCR in accordance with at least one example disclosed herein.

FIG. 33 illustrates a front view of a share screen in accordance with at least one example disclosed herein.

FIG. 34 is a flow diagram illustrating a process of synchronizing ePCR data in accordance with at least one example disclosed herein.

FIG. 35 is a schematic diagram illustrating a mobile computing device in accordance with at least one example disclosed herein.

FIG. 36 is a schematic block diagram of examples of computing and medical device components with which at least one example disclosed herein may be implemented.

FIG. 37A is a schematic block diagram illustrating an example of a logical and physical architecture of an EMS digital assistant as part of an EMS SaaS platform in accordance with at least one example disclosed herein.

FIG. 37B is a schematic block diagram illustrating an example of a logical and physical architecture of a data analytics platform as part of an EMS SaaS platform in accordance with at least one example disclosed herein.

FIG. 37C is a schematic block diagram illustrating an example of a logical and physical architecture of an integration platform in accordance with at least one example disclosed herein.

FIG. 38 illustrates a front view of a chart review screen in accordance with at least one example disclosed herein.

FIG. 39 illustrates a front view of a chronology screen that depicts a synchronized patient encounter timeline in accordance with at least one example disclosed herein.

FIG. 40 illustrates a front view of a medications screen in accordance with at least one example disclosed herein.

FIG. 41 is a flow diagram illustrating another portion of a process of documenting an EMS call within an ePCR in accordance with at least one example disclosed herein.

FIG. 42 illustrates a front view of an insurance screen in accordance with at least one example disclosed herein.

FIG. 43 is a flow diagram illustrating another portion of a process of documenting an EMS call within an ePCR in accordance with at least one example disclosed herein.

### DETAILED DESCRIPTION

As summarized above, the systems and methods disclosed herein are directed to patient charting systems and methods crafted to operate efficiently and reliably in an EMS environment. Often in an emergency encounter, EMS caregivers interact with a critically ill patient for the first time and with no prior medical knowledge about the patient. The emergency encounter is usually in a non-medical environment like a home, office, or gym. In many cases, the encounter occurs in the chaotic environment of a fire scene, a car accident, or a mass casualty scene.

For example, consider an illustrative scenario of a crew of EMS caregivers in an ambulance being called upon to treat a patient suffering from an emergency medical condition (e.g., cardiac arrest, trauma, respiratory distress, drug overdose, etc.) and to transport the patient to a hospital. During the course of this emergency encounter, the EMS caregivers may be required to travel to a patient's scene, determine patient information, such as a mechanism of injury and a chief complaint, observe patient symptoms and conditions, measure patient physiological parameters (such as heart rate and other vital signs, electrocardiogram (ECG) traces, temperature, blood-oxygen data, and the like), administer treatments, interventions, and/or medications, and transport the patient from the scene to a medical facility.

To provide a complete and accurate record of each encounter that includes patient and encounter information, as described above, the EMS caregivers are tasked not only with providing medical care to patients but also recording and documenting detailed encounter information. For example, the EMS caregivers may document observations, examinations, and/or communications with the patient relevant to the patient's medical condition. This patient information can include, for instance, patient biographical information, past medical conditions, medications, allergies, vital signs, mental state, and the like. Other patient information recorded may include patient demographic information and billing/insurance information. In addition to patient information, the EMS caregivers may also be expected to record information regarding the encounter itself, such as the type of service requested, response mode, transport, and the like. This documentation may take the form of an ePCR. The ePCR includes data fields configured to store a comprehensive set of patient and encounter information according to a schema that controls the structure of the data provided to the digital record. The ePCR may include hundreds of mandatory data fields, although data field entries can include “not applicable” for data fields that do not apply to a particular call. In some examples, the schema may be a multi-agency standard that provides a compliance architecture to allow transfer of data and data interoperability between individual agency systems and enables entry of data in a centralized database. An example of such a standard is the National Emergency Medical Services Information Standard (NEMSIS) for emergency care medical record data collection. NEMSIS is an official EMS data collection standard for EMS agencies which allows transfer of data between systems and provides a national EMS repository for reporting and research. NEMSIS provides consistent definitions of data elements used in EMS and other pre-hospital care settings. The NEMSIS data collection via NEMSIS-compliant ePCRs may enable analysis of this data for evaluation of and evidence-based improvements in patient care across an array of EMS agencies. In particular, the NEMSIS-compliant ePCRs conform to a structured XML standard for the ePCR data. NEMSIS and the XML standard are examples only and other formats and/or content requirements are within the scope of this disclosure. For instance, the HL7®FHIR® (Health Level Seven Fast Healthcare Interoperability Resources) standard defines how healthcare information can be exchanged between different computer systems such as those servicing emergency care and those servicing hospitals. Other examples of standards include, but are not limited to, an HL7 version 2, version 3 or CDA standard, an Electronic Data Interchange (EDI) Healthcare including, 270, 271, 276, 277, 278, 820, 834, 835, 837P and 837I standard, SNOMED CT standard, diagnosis classification ICD standard, and procedure chart HCPCS and CPT standards.

In an implementation, the data fields within an ePCR may be organized into data set sections that cover various aspects of the emergency encounter. These data set sections may include, for example, data sets for airway, cardiac arrest, EMS crew, medical device, dispatch, patient disposition, patient examination, patient history, injury, laboratory results, and medications. In addition, in some implementations, an agency may include customized data set sections. As an example, a patient history section may include the data fields indicated below in Table 1. Examples of field values for the data fields are also provided in Table 1. The data field values may be associated with an ICD chart (e.g., International Classification of Diseases) for billing purposes.

TABLE 1

| Data field | Field value |
|---|---|
| Last name of Patient's Practitioner | Smith |
| First name of Patient's Practitioner | Chris |
| Advance Directives | none |
| Medication Allergies | Penicillin |
| Environmental/Food Allergies | Peanut |
| Medical/Surgical History | Type 2 diabetes |
| Medical History Obtained From | Patient's husband |
| Patient's Immunization | Flu |
| Immunization Year | Current year |
| Current Medications | Metformin |
| Current Medication Dose | 500 |
| Current Medication Dosage Unit | mg |
| Current Medication Administration | Oral |
| Alcohol/Drug Use | none |
| Pregnancy | yes |
| Last Oral Intake | none |

As another example of ePCR data, Table 2 below shows examples of data fields and data field values for a pre-scheduled dialysis transport.

TABLE 2

| Data field | Field value |
|---|---|
| Call Source | Phone call |
| Dispatch Center | Verifast EMS Services |
| Run Number | 47 |
| Incident Number | 56-87 |
| Dispatched Complaint | Palliative Care |
| Patient Acuity at Dispatch | Priority 4 (Non-Acute) |
| Changed Priority | Pre-Scheduled |
| Trauma call type | Medical and trauma |
| Call type | BLS |
| Response Mode | Pre-Scheduled |
| Additional Response Mode | No lights or Sirens |
| Pickup Zone | 16 |
| Response Delay | None |
| Type of Service | Interfacility transport |
| Patient Disposition | Treated & transported |

For maximum accuracy and to provide the most real-time benefit to caregivers, both pre-hospital and at a transport destination, the ePCR should be completed contemporaneously with, i.e., during, the ongoing encounter, with a completed document available upon transfer of the patient from EMS to a medical facility. However, this documentation competes for the attention of the responders with the responders need to attend to medical care of the patient. For example, entering this data during the encounter may divert the attention of the EMS caregiver away from the patient and reduce the amount of time the EMS caregiver can devote to patient care. This is particularly true if the documentation process relies on data entry via a graphical user interface (GUI) that requires lengthy hands-on data entry and manual navigation through a complex and hierarchical set of data entry screens. For example, data entry to a computing device, such as a tablet, laptop, or other mobile device processing the ePCR may require keyboard entries of information in an alphanumeric format and/or menu entries from lengthy and complex nested menu structures. In addition, a multi-layered and hierarchical nature may not conform to a natural medical workflow for the responders and thus may interrupt and disrupt this workflow. This aspect of ePCR screens can make it time consuming and difficult to enter patient and encounter information. In some implementations, the ePCR may include 50-1000 fields for which a data entry is required (e.g., required by laws of a state or another jurisdiction and/or required for adherence to a data collection standard). The voluminous number of required fields and layout of the GUI may cause users to skip or rush through these fields, particularly in the context of an emergency response. A dedicated documentarian is typically not available in a small team of EMS responders. However, skipped, inaccurate, and/or incomplete data entry may negatively affect patient care and patient outcomes. Furthermore, such reduction or inaccuracy may result in a reduction in the accuracy and completeness of information passed from an initial emergency care encounter to a subsequent hospital encounter. For example, to provide timely medical care, a responder may resort to recordation short-cuts, such as writing notes on scrap paper, backs of gloves, ECG tape, or other readily available handwriting stock and/or may delay documentation until the conclusion of an encounter. Post hoc completion of the ePCR increases inaccuracies and introduces delay into the overall continuity of care provided to the patient because this practice requires the EMS caregiver to remember what transpired during the encounter and, in some instances, what portions of the ePCR have and have not been completed.

As an additional matter, the computing device used for data entry may be too large, bulky, or simply inconvenient to carry and utilize in view of the many treatment devices and other essential medical equipment required (e.g., defibrillators, patient monitors, ventilation equipment, trauma kits, medications, gurneys, back boards, etc.). Furthermore in many situations, the computing device may lack network connectivity or be vulnerable to sporadic connectivity. For example, EMS care provided in a rural area, in a parking garage, in an urban canyon, or during transport may be subject to unavailable or unreliable network connectivity. This may be a problem if multiple responders are trying to complete different portions of the ePCR based on their caregiving role.

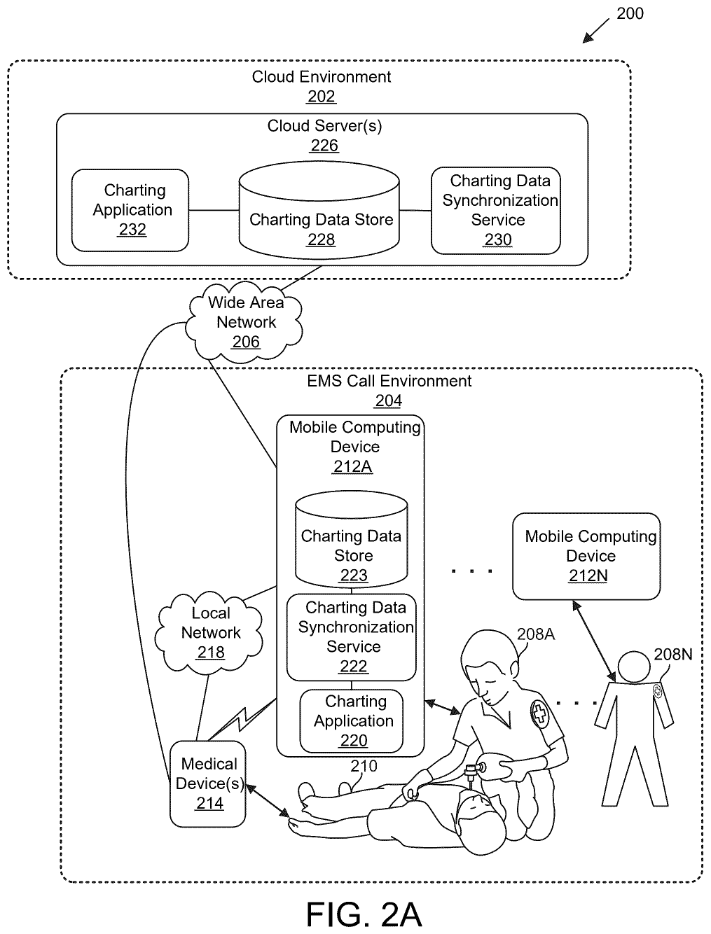

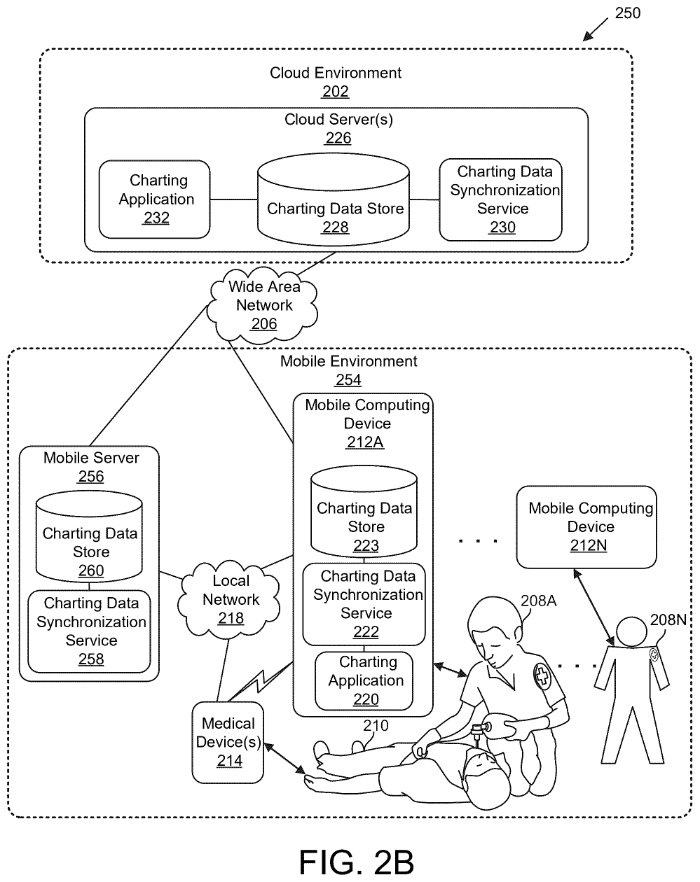

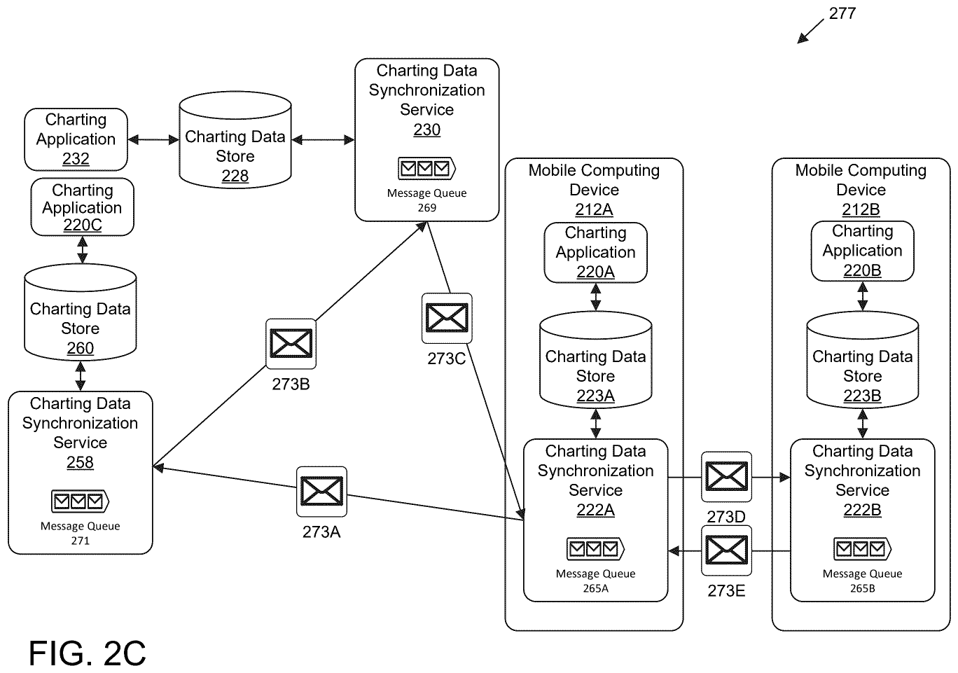

Thus, and in accordance with at least some examples disclosed herein, an EMS charting system is provided that minimizes disruptions to a medical workflow and is available without network connectivity. Furthermore, the EMS charting system disclosed herein is available on a smartphone device, thus relieving responders of the need to carry and tend to additional computing devices amongst other advantages. The EMS charting system addresses the issues articulated above, among others, through implementation of a unique combination of features. For example, in some implementations, the EMS charting system comprises a mobile computing device configured to host a mobile EMS charting application (e.g., the mobile EMS charting application 220, as shown in FIGS. 2A-2C). A particular instance of the mobile EMS charting application may be configured to communicate with other instances of the mobile EMS charting application hosted by other mobile computing devices and/or with data synchronization services hosted by edge and/or cloud servers. An example of a first instance of the mobile EMS charting application 200A communicating with a second instance of the mobile EMS charting application 200B is described further below with reference to FIG. 2C. Each mobile EMS charting application, when executed by its host mobile computing device, may implement a GUI configured to receive ePCR data, a local data store configured to house the ePCR data, and a data synchronization service configure to interoperate with data synchronization services hosted by other computing devices within the EMS charting system. In some examples, the data stores and synchronization services implemented within the EMS charting system enable seamless operation of the mobile EMS charting application even where the host mobile computing devices and/or servers experience degraded or lost network connections.

In some examples, the GUI implemented by the mobile EMS charting application includes a set of screens that are tailored to easily, quickly, conveniently, and accurately record ePCR data. These interface screens can be rendered, for example, via a touchscreen of a mobile computing device executing the mobile EMS charting application. In these examples, the mobile EMS charting application is configured to enable efficient data entry under dynamic field conditions, even when implemented on resource-constrained mobile computing devices, such as smartphones. For instance, in certain examples, the mobile EMS charting application renders the screens in a simplified and flattened topology relative to the topology of traditional patient charting interfaces which are designed to leverage the more expansive screen space afforded larger mobile computing devices, such as tablets or laptops. In various implementations, a portion or all of the EMS charting system features disclosed herein may be available on a mobile and/or display device other than a smartphone, such as a medical device display, a tablet computing device, a watch computing device or other wearable computing device, a heads-up glasses display, an augmented reality display device, and/or a virtual reality display device. Where one of these devices is a primary device associated with an EMS caregiver, the advantage of not carrying additional devices is realized. Additionally, where these other mobile and/or display devices may have limited display space, the advantages described herein regarding the simplified and flattened topology may enable these devices to provide a charting application without relying on more expansive screen space. Further, in some examples, the GUI comprises additional features, one-touch, touchscreen gesture data entry and navigation controls that enable efficient data entry under dynamic field conditions. In combination, the simplified screen topology and data entry and navigation features decrease the number of interactions between an EMS caregiver and a mobile computing device that are required to document a patient encounter. Moreover, since the mobile EMS charting application can implement the GUI on resource-constrained mobile computing devices, some examples of the EMS charting system utilize smartphones routinely carried by, and readily accessible to, EMS caregivers. This feature increases the availability of the mobile EMS charting application to the EMS caregivers. Some implementations can additionally control the smartphone's integrated camera to provide scanning capabilities to receive certain ePCR data (e.g., medical and/or demographic information). In certain examples, the EMS charting applications hosted by resource-constrained mobile computing devices are configured to transfer ePCR data to a tablet or laptop to enable EMS caregivers to complete an ePCR on the larger form factor device. In some examples, the mobile EMS charting application is configured to enable data entry from multiple smartphone devices associated with a patient encounter scene and synchronize that data entry in the absence of continuous network connectivity.

Examples of the EMS charting system described herein enable EMS caregivers to record ePCR data using resource-constrained mobile computing devices, such as a smartphone without an Internet connection. This technical advantage is critical in practice where the scene of an emergency may lack Internet connectivity (e.g., a rural highway, a parking garage, an individual residence, etc.). Additionally, given the currently ubiquitous nature of smartphones, an EMS caregiver may record information using a readily accessed and familiar device. Recordation of this information provides for a host of benefits including, for example, improved interventions by the EMS caregiver, improved continuity of patient care between the EMS caregiver and other EMS caregivers, improved compliance with medical protocols, more efficient operation of an EMS crew due to distributed and contemporaneous documentation, and more efficient operation of the employer of the EMS caregiver, to name a few.

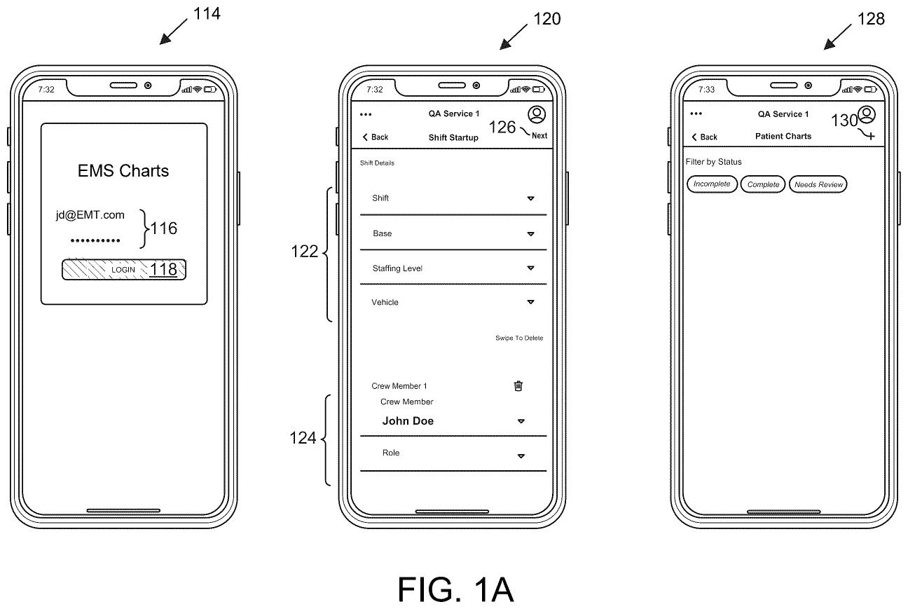

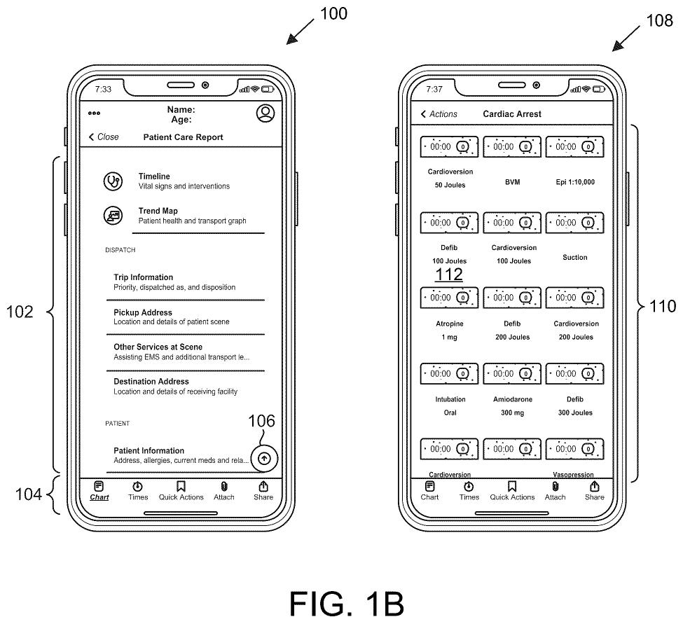

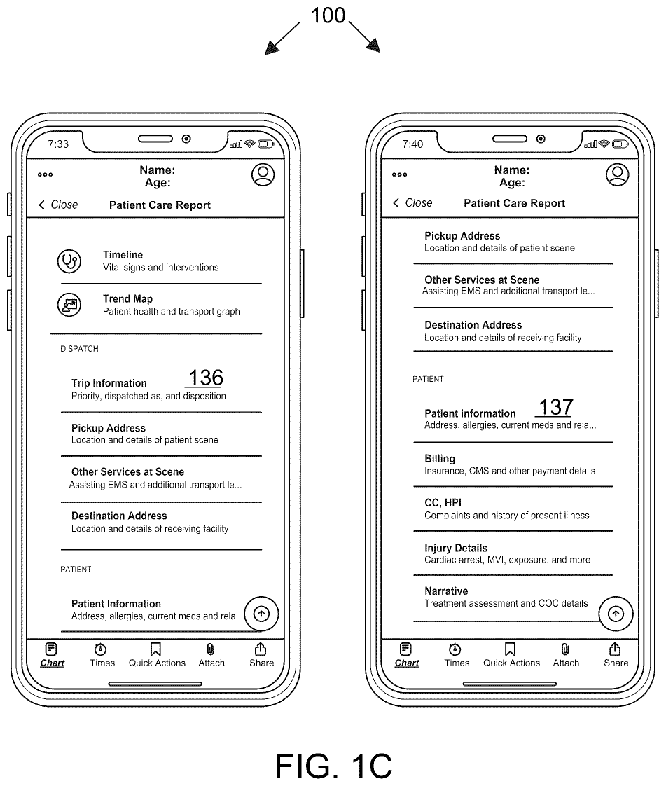

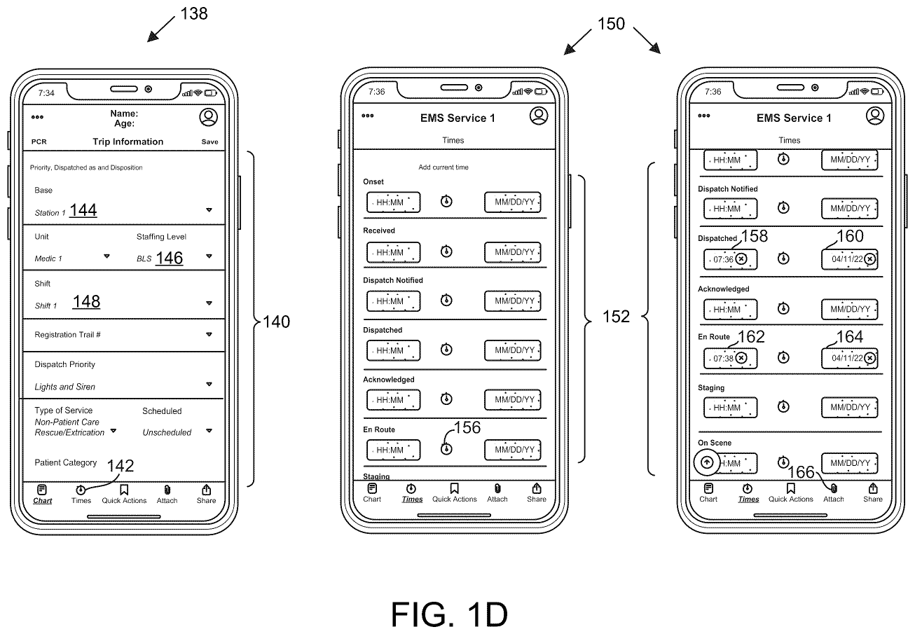

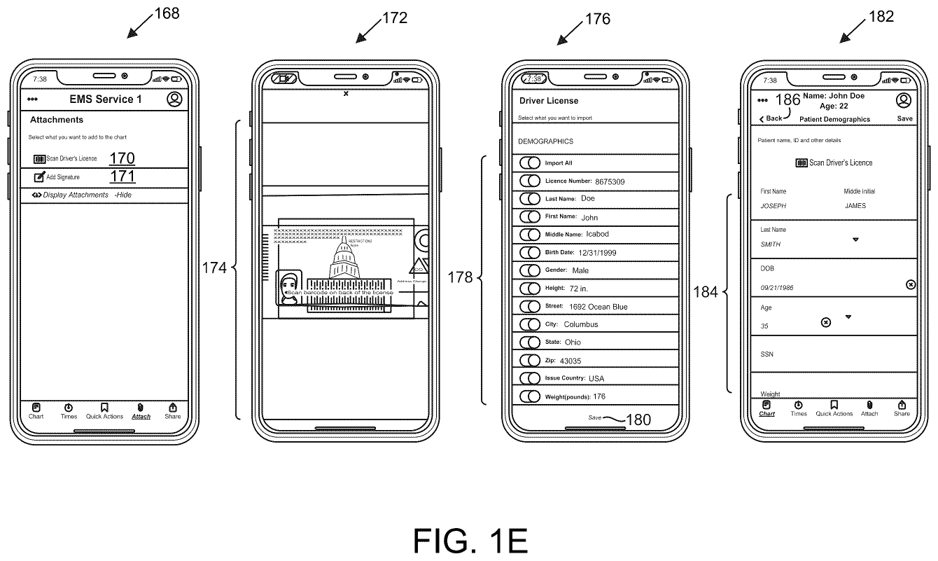

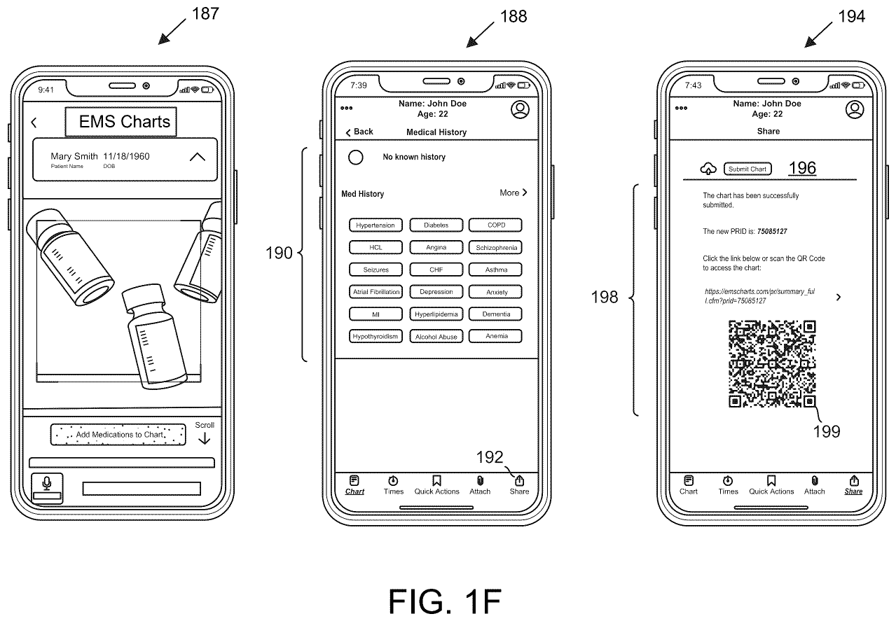

By way of introduction, FIGS. 1A-1F illustrate screens of a GUI implemented by the mobile EMS charting application in some examples. As can be seen in review of FIGS. 1A-1F, the design of the mobile EMS charting application reflects a mobile-first philosophy. In addition, the mobile EMS charting application leverages mobile device hardware and technology to collect and input data in an offline mode (e.g., when its host device is not connected to a network) and an online mode (when the host device is connected to a network). In at least some examples, the mobile EMS charting application is configured to tightly integrate, via the host device's web browsing capabilities, with a cloud-based mobile EMS charting application to receive configuration information from, and to submit ePCR data to, the cloud-based application. Additionally, the mobile EMS charting application streamlines and/or automates data collection at least through scanning and/or optical character recognition (OCR) capabilities, importing computer aided dispatch (CAD) data, and providing QA buttons, which are described further below. In some examples, the mobile EMS charting application also incorporates electronic signatures.

In certain examples, the mobile EMS charting application is configured to control its host device to render and interact with a user via the GUI screens illustrated in FIGS. 1A-1F. The screens illustrated in FIGS. 1A-1F support workflows routinely performed by users, such as EMS caregivers, in the field. FIG. 1A illustrates a login screen 114, a shift startup screen 120, and a chart selection screen 128.

According to one workflow, a user logs into the mobile EMS charting application at the start of a shift. The screen 114 supports this operation. As shown in FIG. 1A, the screen 114 includes security credential controls 116 and a login control 118. In this example, the mobile EMS charting application is configured to receive input specifying a user identifier and a password via the security controls 116 and to attempt authentication of a user associated with the user identifier and the password in response to receiving input selecting the login control 118. In some examples, to authenticate the user the mobile EMS charting application may interoperate with an identity provider that is local to the host device or implemented by an edge or cloud server in communication with the host device. Alternatively or additionally, the mobile EMS charting application may utilize one or more biometric sensors (e.g., a fingerprint scanner) embedded within the host device to authenticate the user.

Continuing with the example workflow, the user enters shift information including shift detail and crew member information. The shift startup screen 120 supports this operation. In some examples, the mobile EMS charting application is configured to control the host device to render the screen 120 if the attempt to authenticate the user is successful. As shown in FIG. 1A, the screen 120 includes shift detail controls 122, crew member controls 124, and a next screen control 126. The shift detail controls 122 include a shift control, a base control, a staffing level control, and a vehicle control. The crew member controls 124 include a crew member control and a role control. In this example, the mobile EMS charting application is configured to receive input specifying shift and crew member information via the controls 122 and 124. The mobile EMS charting application is further configured to store the shift and crew member information in a local data store. Further, in this example, the EMS charting device is configured to control the host device to render the chart selection screen 128 in response to receiving input selecting the next screen control 126.

Continuing with the example workflow, the user proceeds to enter ePCR data. The mobile EMS charting application controls the host device to render a new chart screen 100 in response to receiving input selecting the add control 130 illustrated in FIG. 1A. FIG. 1B illustrates the screen 100 and a quick action (QA) screen 108. The mobile EMS charting application 220 may provide QA screens for one or more encounter types. In some examples, the mobile EMS charting application is configured to provide QA screens with controls associated with actions that are most likely to be performed by the EMS caregiver to address the chief complaint of the EMS call according to protocol. It should be noted that the QA screens are customizable by EMS agency. This feature enables a medical director of the agency to configure and customize the QA screens to comply with agency specific protocols.

In the example shown, the QA screen 108 is directed to cardiac arrest as an example only and is not limiting of the disclosure. In certain examples, the mobile EMS charting application is configured to control its host device to render screens and interact with a user (e.g., an EMS caregiver) via the screens 100 and 108. The screen 100 includes several section controls 102, a navigation bar 104, and a top control 106. Each of the section controls 102 may be referred to herein individually as a section control 102. The mobile EMS charting application is configured to detect user interaction with the host device via the screen 100, and to respond thereto. Such user interaction with the host device may take the form of one or more touchscreen gestures. Such gesture input may include swipes, taps, pinches, shakes and other input generated by interaction of the human hand directly with a touchscreen or a device including the touchscreen. For instance, the mobile EMS charting application may be configured to detect a swipe (e.g., up or down) over the section controls 102 and to respond to the detected swipe by scrolling the section controls 102 in the direction of the swipe. The mobile EMS charting application may be further configured to detect selection (e.g., a touch or tap) of an individual section control 102 and to respond to the selection by opening a section of the ePCR associated with the individual section control 102. Similarly, the mobile EMS charting application may be configured to detect selection of a control within the navigation bar 104 and to respond to the selection by opening a screen associated with the selected control. Further, in some examples, the mobile EMS charting application is configured to detect selection of the top control 106 and to respond to the selection by scrolling the section controls 102 downward until the top of the screen 100 is displayed.

The QA screen 108 includes QA controls 110. Each of the QA controls 110 may be referred to herein individually as a QA control 110. The mobile EMS charting application may be configured to detect selection of an individual QA control 110 and to respond to the selection by recording ePCR data associated with the individual QA control 110. For instance, the mobile EMS charting application may record ePCR data indicating that the patient was defibrillated in response to selection of the defib control 112, as a specific example of an individual QA control 110. It should be noted that, in each instance described above, the mobile EMS charting application is configured to detect and respond to user interactions that may be performed with one hand only (e.g., the user's thumb). Such one-handed operation can be particularly beneficial during an EMS encounter as it frees the responder's other hand for medical tasks.

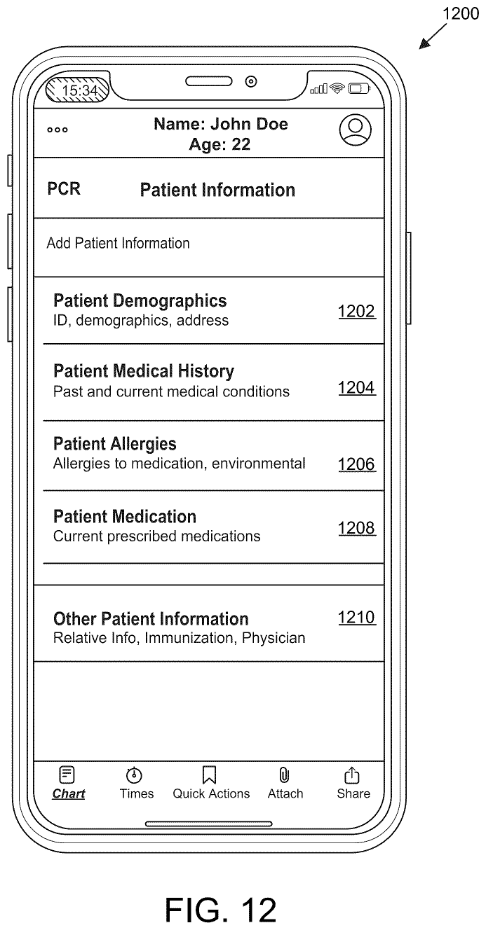

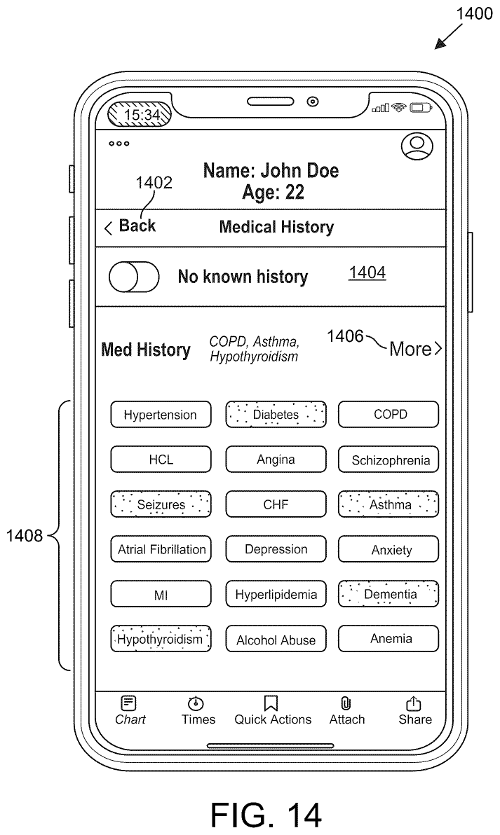

As shown in FIG. 1C, the new chart screen 100 includes a variety of controls that enable the user to navigate to various sections of the new ePCR. These section controls include the trip information control 136 and the patient information control 137. In this example, the mobile EMS charting application is configured to render a trip information screen in response to receiving input selecting the trip information control 136. Further, in this example, the mobile EMS charting application is configured to render a patient information screen in response to receiving input selecting the patient information control 137. This patient information screen includes controls configured to access screens associated with sections of an ePCR associated with patient demographic information, allergies, medications, and medical history. One example of a patient information screen is described further below with reference to FIG. 12. One example of a medical history screen accessible via the patient information screen is described further below with reference to FIG. 14.

Continuing with the example workflow, the user enters trip information within the dispatch section of the ePCR. FIG. 1D illustrates a trip information screen 138 that supports this operation. In some examples, the mobile EMS charting application controls the host device to render the screen 138 in response to receiving input selecting the trip information control 136 illustrated in FIG. 1C. As illustrated in FIG. 1D, the screen 138 includes trip information controls 140 and a time control 142 within a navigation bar. In this example, the trip information controls 140 include a base control 144, a staffing level control 146, and a shift control 148. In this example, the mobile EMS charting application is configured to populate the trip information controls (e.g., the controls 144, 146, and 148) with default values based on the information received via the shift screen 120 described above with reference to FIG. 1B. In this example, the mobile EMS charting application is configured to receive input specifying trip information via the trip information controls 140, and to store the trip information as ePCR data in a local data store. Also, in this example, the mobile EMS charting application is configured to render a times entry screen in response to receiving input selecting the time control 142. Additionally or alternatively, in some examples, at least a portion of the trip information displayed within the trip information controls may be pre-populated from dispatch information imported from a CAD. In such examples, the mobile EMS charting application may automatically pre-populate one or more data fields with the data imported from the CAD. In various implementations, the mobile EMS charting application may request confirmation of the pre-populated information from the user and/or may apply adjudication rules to give preference to pre-populated information, user entry information, or scanned information at the point of care.

Continuing with the example workflow, the user records the date and time that the user was dispatched and the date and time that the user commenced travel. FIG. 1D illustrates a times entry screen 150 that supports these operations. In some examples, the mobile EMS charting application controls the host device to render the screen 150 in response to receiving input selecting the time control 142. As illustrated in FIG. 1D, the screen 150 includes time and date controls 152 and an attach control 166 in a navigation bar. In this example, the time and date controls include a current timestamp control 156, a dispatched time control 158, a dispatched date control 160, an en route time control 162, and an en route date control 164. The mobile EMS charting application may be configured to receive input specifying dates and times of named events via the time and date controls 152 and to store the specified dates and times in association with the named events as ePCR data in a local data store. For instance, the mobile EMS charting application may be configured to receive input specifying a time in the recent past via the dispatched time control 158 and today's date via the dispatched date control 160. Additionally, the mobile EMS charting application may be configured to set the en route time control 162 and the en route date control 164 to the current time and date in response to receiving input selecting the current timestamp control 156. The current timestamp control 156 provides a timestamp to an event in response to a single touchscreen gesture. Also, in this example, the mobile EMS charting application is configured to render an attachment screen in response to receiving input selecting the attach control 166.

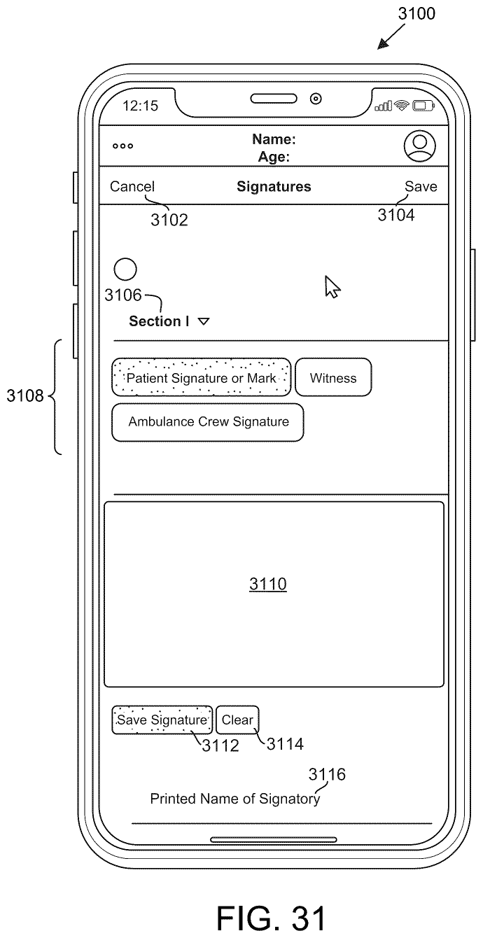

Continuing with the example workflow, the user arrives at the scene of the service call and collects information and signatures from the patient. FIG. 1E illustrates an attachments screen 168 and a scan screen 172 that collectively support some of these operations. The screen 168 enables the user to obtain the patient's signature. The screen 172 enables the user to obtain a driver's license image for the patient and scan the driver's license to acquire demographic information regarding the patient. In an implementation, the screen 172 may enable the user to obtain an image of and/or scan patient identification information other than a driver's license, such as, for example, a passport or other government issued identification document, a social security card, a national healthcare card or bracelet, an insurance coverage card or document, etc. In some examples, the mobile EMS charting application controls the host device to render the screen 168 in response to receiving input selecting the attach control 166 illustrated in FIG. 1D. As illustrated in FIG. 1E, the screen 168 includes a scan control 170 and a signature control 171. In this example, the mobile EMS charting application is configured to render a signature screen in response to receiving input selecting the signature control 171. The signature screen may include controls configured to acquire a touchscreen signature from the patient. One example of a signature screen 3100 is described further below with reference to FIG. 31. In addition, in this example, the mobile EMS charting application is configured to render the scan screen 172 in response to receiving input selecting the scan control 170. As shown in FIG. 1E, the scan screen 172 includes a viewfinder control 174. The mobile EMS charting application is configured to access a camera of the host device and control the host device to display images acquired via the camera in the viewfinder control 174. Additionally, in some examples, the mobile EMS charting application is configured to identify and decode a barcode or some other fiducial (e.g. a QR code) depicted in an acquired image. In these examples, the mobile EMS charting application is configured to store demographic information successfully decoded from a fiducial as ePCR data in a local data store. As shown on screen 187 in FIG. 1F, the EMS charting application may scan a bar code of a medication. Other information that can be acquired via scanning and optical character recognition, in various examples, includes freeform typed or handwritten text that specifies ePCR data (e.g., face sheets, pdfs, etc.). Other files that may be attached to the ePCR, in some examples, include image files, pdf files, medical device electronic case files, medical history and analysis, and files containing health and/or activity information collected by a smartphone app, such as the Health app available on iOS devices.

Continuing with the example workflow, the user selects patient demographic information to import into the ePCR. FIG. 1E illustrates a importation screen 176 for a driver's license or other patient identification information that supports this operation. In some examples, the mobile EMS charting application controls the host device to render the screen 176 in response to scanning and decoding a fiducial as illustrated in screens 168 and 172. As illustrated in FIG. 1E, the screen 176 includes demographic selection controls 178 and a save control 180. In this example, the mobile EMS charting application is configured to receive input toggling selections of demographic items via the selection controls 178. The selection controls 178 enable the user to specify which demographic items to import and which to omit due to error or based on other considerations. For example, if the decoded fiducial specifies an old address because the patient moved after the driver's license or other patient identification documentation was issued, the selection controls enable the user to prevent importation of the old address. Further, in this example, the mobile EMS charting application is configured to receive input selecting the save control 180 and, in response, to import demographic information associated with selected controls 178 and save the imported demographic information as ePCR data in a local data store.

Continuing with the example workflow, the user reviews the demographic information imported into the ePCR. FIG. 1E illustrates a patient demographics screen 182 that supports this operation. In some examples, the mobile EMS charting application controls the host device to render the screen 182 in response to receiving input selecting the save control 180. As illustrated in FIG. 1E, the screen 182 includes demographic item controls 184 and a back control 186. In this example, the mobile EMS charting application is configured to display patient demographic data and interact with the user to maintain the same via the controls 184. Further, in this example, the mobile EMS charting application is configured to receive input selecting the back control 186 and, in response, to render a chart screen (e.g., the chart selection screen 100 of FIG. 1C).

Continuing with the example workflow, the user enters the patient's medical history into the ePCR. FIG. 1F illustrates a medical history screen 188 that supports this operation. In some examples, the mobile EMS charting application controls the host device to render the screen 188 in response to receiving input selecting the back control 186 to access the screen 100 of FIG. 1C, receiving input selecting the patient information control 137 of FIG. 1C, and then receiving input selecting a medical history control. As illustrated in FIG. 1F, the screen 188 includes history item controls 190 and a share control 192 in a navigation bar. In this example, the mobile EMS charting application is configured to interact with the user to collect the patient's medical history via the controls 192. For instance, in the example shown, the mobile EMS charting application is configured to receive input toggling selection of the history item controls 190 and, in response thereto, store or delete associations between the patient and selected history items as ePCR data in a local data store. Further, in this example, the mobile EMS charting application is configured to receive input selecting the share control 192 and, in response, to render a share screen.

Continuing with the example workflow, in an implementation, the user of the mobile EMS charting application 220 may save the ePCR, either locally or on a remote server. The mobile EMS charting application 220 may associate the saved ePCR with an identifier and provide that identifier at the mobile device to enable the user of the mobile EMS charting application 220 to share the ePCR with a third party. For example, the mobile EMS charting application 220 may represent the identifier as a quick response (QR) code 199. The third party may be, for example, another caregiver within an EMS team or personnel at a receiving medical facility (e.g., a member of an emergency room staff). In an implementation, the user of the mobile EMS charting application 220 may share the QR code when handing the patient off to a second crew that assumes control of patient care. FIG. 1F illustrates a share screen 194 that supports this operation. In some examples, the mobile EMS charting application controls the host device to render the screen 194 in response to receiving input selecting the share control 192. As illustrated in FIG. 1F, the screen 194 includes a submit control 196 and share controls 198. In this example, the mobile EMS charting application is configured to receive input selecting the submit control 196 and, in response thereto, to upload the current ePCR stored in local storage to a cloud server for further processing. As described below with reference to FIGS. 2A and 2B, upload of the current ePCR may or may not involve transmission of the current ePCR to an edge server. Further, in this example, the mobile EMS charting application is configured to display information identifying the submitted ePCR via the share controls 198. As shown, this identification information can include a serial number and/or a uniform resource locator (URL) rendered as text or a fiducial (e.g., the quick response code 199).

### EMS Charting System

FIGS. 2A and 2B illustrate example EMS charting system implementations in accordance with some examples. As shown in FIG. 2A, the EMS charting system 200 includes a cloud environment 202, an EMS call environment 204, and a wide area network 206 through which computing devices in the cloud environment 202 and the EMS call environment 204 can communicate and interoperate with one another. In some examples, the cloud environment 202 includes one or more cloud server(s) 226 that host a cloud EMS charting application 232, and a charting data store 228. In an implementation, the cloud environment 202 may include a charting data synchronization service 230. In some examples, the EMS call environment 204 includes a single mobile computing device 212A provisioned with the mobile EMS charting application 220. In this case, one or more caregivers may document a single ePCR via a single mobile computing device. In other examples, the EMS call environment 204 includes a plurality of mobile computing devices 212A-212N provisioned with the mobile EMS charting application 220. In an implementation, multiple caregivers may utilize separate devices to access and populate a same ePCR. As described below, this multiple caregiver access and documentation may occur with or without connectivity to the cloud environment 202. The EMS call environment may further include one or more medical device(s) 214 that are configured to communicatively couple with one another directly or indirectly via, for example, a local network 218. The mobile computing devices 212A-212N are associated with and used by one or more EMS caregivers 208A-208N. In some examples, each EMS caregiver of the one or more EMS caregivers 208A-208N is associated with a single, respective mobile computing device of the mobile computing devices 212A-212N. In certain examples, the association between an EMS caregiver and a mobile computing device is established via the EMS caregiver successfully authenticating with the EMS charting system via the mobile computing device. Also as shown in FIG. 2A, each of the medical device(s) 214 is associated with a respective patient 210 and is configured to monitor and/or treat the patient 210. It should be appreciated that the number of patients within a given EMS call environment 204 may be more than one. The one or more caregivers 208A-208N may be associated with a single EMS agency crew assigned to the EMS call environment to provide a medical response for one or more patients 210. In some situations, the responders in a crew of two or more responders may have different roles. For example, a crew may include a paramedic, an emergency medical technician (EMT), and an emergency medical responder (EMR). The paramedic has a wider scope of practice than an EMT and may perform interventions such as delivery of medications, either orally or intravenously, or intubation. The EMT may manage delivery of CPR and the EMR may supervise loading the patient onto a gurney and may drive the ambulance. In an example, each of these crew members may utilize the mobile EMS charting application 220 to document the portion of the same ePCR for the patient encounter that is relevant to the tasks within their scope of practice.

In some examples, devices within the cloud environment 202 and the EMS call environment 204 (and the processes that these devices implement) can communicate with one another in real-time, thereby enabling rapid information exchange and consolidation while the patient 210 is being monitored and/or treated. Thus, for example, each of the mobile computing devices 212A-212N and/or the cloud server(s) 226 may receive and process data generated by the mobile computing devices 212A-212N or the medical device(s) 214 while the EMS call is happening, so that users (e.g., the EMS caregivers 208A-208N) of the mobile computing devices 212A-212N or of the cloud server(s) 226 are able to see events unfold in real-time. In such a scenario, the charting data synchronization service may orchestrate synchronization of data entries at each of multiple mobile devices 212A-212N.

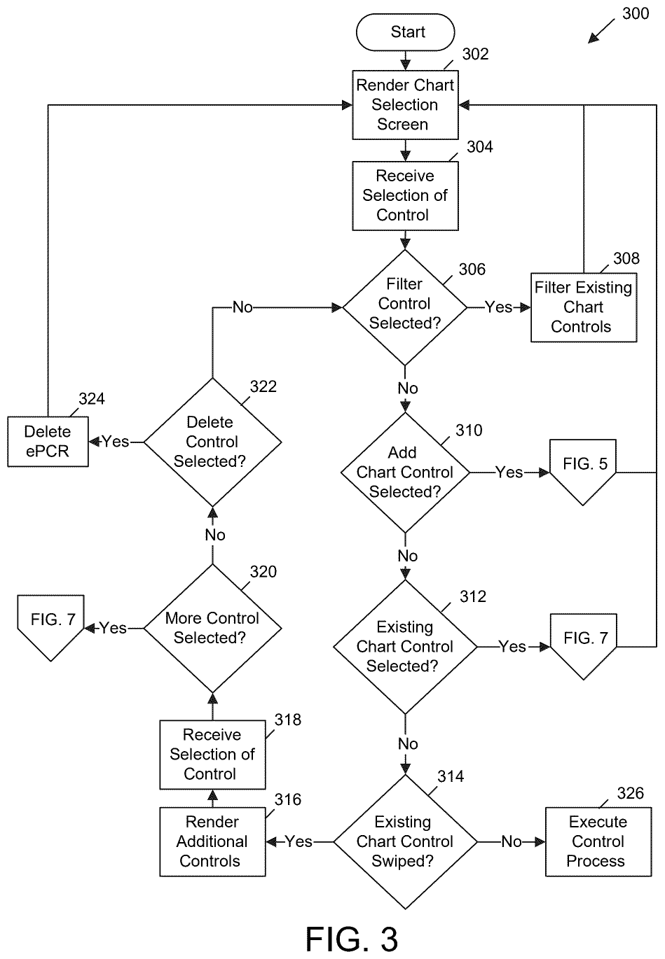

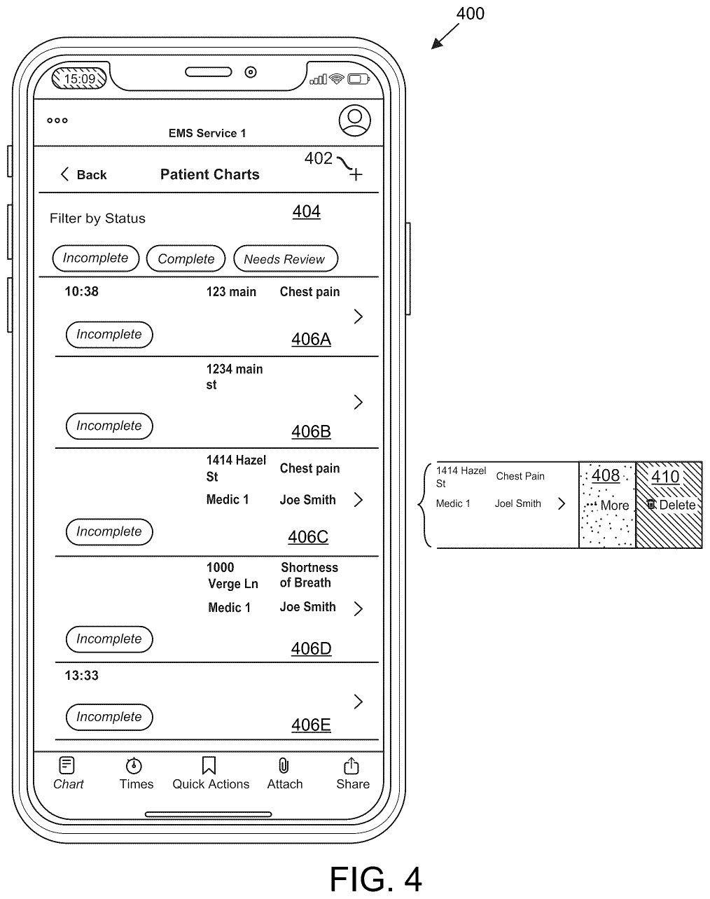

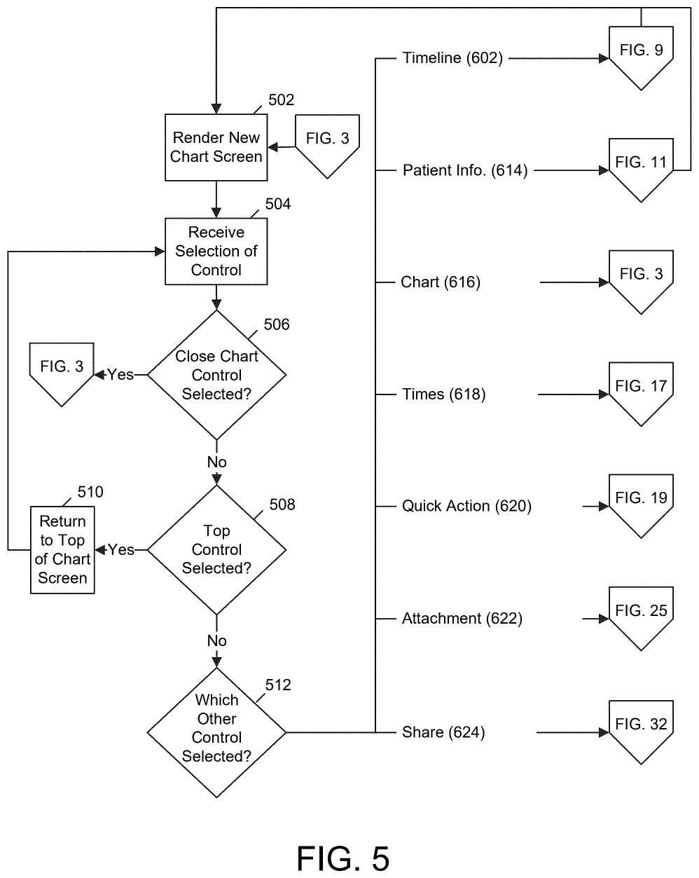

In some examples of the EMS call environment 204, each mobile EMS charting application 220 is configured to interact with a user (e.g., one of the EMS caregivers 208A-208N) to review, generate, and/or manipulate existing ePCR data through a streamlined GUI including a set of screens designed for small screen sizes (e.g., screens having a diagonal dimension of approximately 19.5 cm or less and/or displays associated with portable computing devices that are designed to be held and be controllable with a same and single hand). For example, a smartphone may include a display of a small display size. Screens provided by the mobile EMS charting application 220 include one or more user interface controls that are configured to receive input data and/or display output data. The mobile EMS charting application 220 is configured to receive input selecting the user interface controls and to execute, in response to receiving a selection of any given control, a process associated with the control. Many of the screens and controls described herein implement innovative features that increase the efficiency of the EMS caregivers 208A-208N in documenting emergency care. Examples of some of these screens are described further below with reference to FIGS. 4, 6A, 6B, 8, 10, 12, 14, 16, 18, 20, 22, 24, 26, 28, 29, 31, and 33. Example processes that each mobile EMS charting application 220 is programmed to execute in this configuration are described further below with reference to FIGS. 3, 5, 7, 9, 11, 13, 15, 17, 19, 21, 23, 25, 27, 30, and 32. In certain examples, the mobile EMS charting application 220 is implemented as a stand-alone, native app configured to run under the operating system of its host device. Alternatively or additionally, the mobile EMS charting application 220 can be served to its host device as a browser-enabled application from the cloud EMS charting application 232. In an implementation, one or more of the mobile computing devices 212A-212N may include a larger form factor display than that of, for example, a smartphone. For example, one or more of the devices 212A-212N may be a laptop computer or a tablet computer. In such an implementation, the cloud EMS charting application 232 is configured to interact with (e.g., receive input from or provide output to) a user to review, generate, and/or manipulate existing ePCR data through a GUI including a set of screens designed for larger display sizes (e.g., displays having a diagonal dimension of greater than approximately 19.5 cm and/or displays associated with portable computing devices that are not designed to be held and be controllable with a same and single hand).

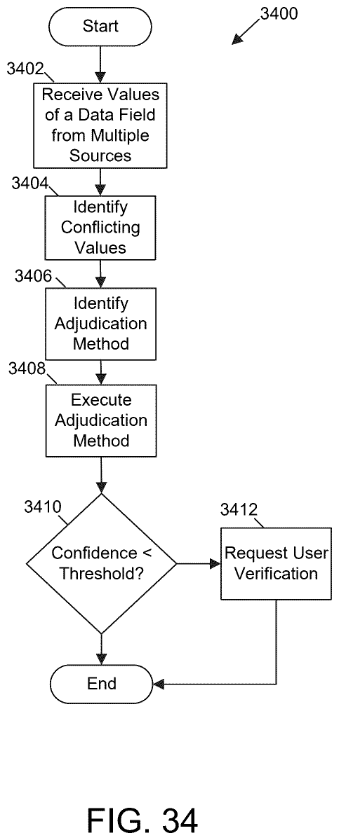

In some examples, the synchronization service 230 is configured to interoperate (e.g., via a set of application programming interface (API) calls) with other instances of the synchronization service (e.g., each synchronization service 222) to maintain a common view of the ePCR data stored within the EMS charting system 200 for all EMS charting applications (e.g., each EMS charting application 220) and the EMS charting application 232. In some examples, when processing these API calls, the synchronization service 230 transmits, receives, and processes synchronization messages that indicate each change made to ePCR data, a timestamp of the change, and an identifier of the originator of the change (e.g., an automated process, a user, etc.). Example processes that the synchronization service 230 is programmed to execute in this configuration are described further below with reference to FIG. 34. The charting data store 228 is configured to store ePCR data for manipulation by the EMS charting application 232, and/or the synchronization service 230. Each synchronization service 222 is configured to interoperate (e.g., via a set of API calls) with the synchronization service 230 and synchronization services 222 on other host devices to maintain a common view of the ePCR data stored within the EMS charting system 200 for all mobile EMS charting applications 220 and the cloud EMS charting application 232. In some examples, when processing these API calls, each synchronization service 222 transmits or receives, and processes, synchronization messages that indicate each change made to ePCR data, a timestamp of the change, and an identifier of the originator of the change (e.g., an automated process, a user, etc.). Example processes that each synchronization service 222 is programmed to execute in this configuration are described further below with reference to FIG. 34. Each charting data store 223 is configured to store ePCR data for manipulation by a mobile EMS charting application 220 and/or a synchronization service 222 hosted locally to (e.g., on the same mobile computing device as) the charting data store 223.

Each of the charting data stores 223 and/or the charting data store 228 may be organized according to a variety of physical and/or logical structures. In at least one example, the data stores 223 and/or 228 are implemented within a relational database having a highly normalized schema and accessible via a structured query language (SQL) engine, such as ORACLE or SQL-SERVER. In some examples, at least portions of the data stores 223 and 228 are implemented as flat operating system files including serialized, proprietary data structures. Thus, the data stores 223 and 228 as described herein are not limited to a particular implementation.

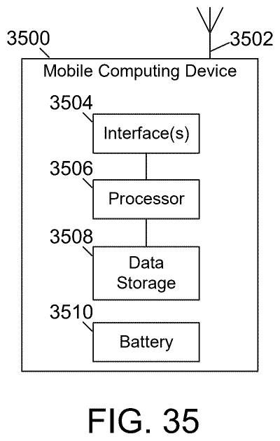

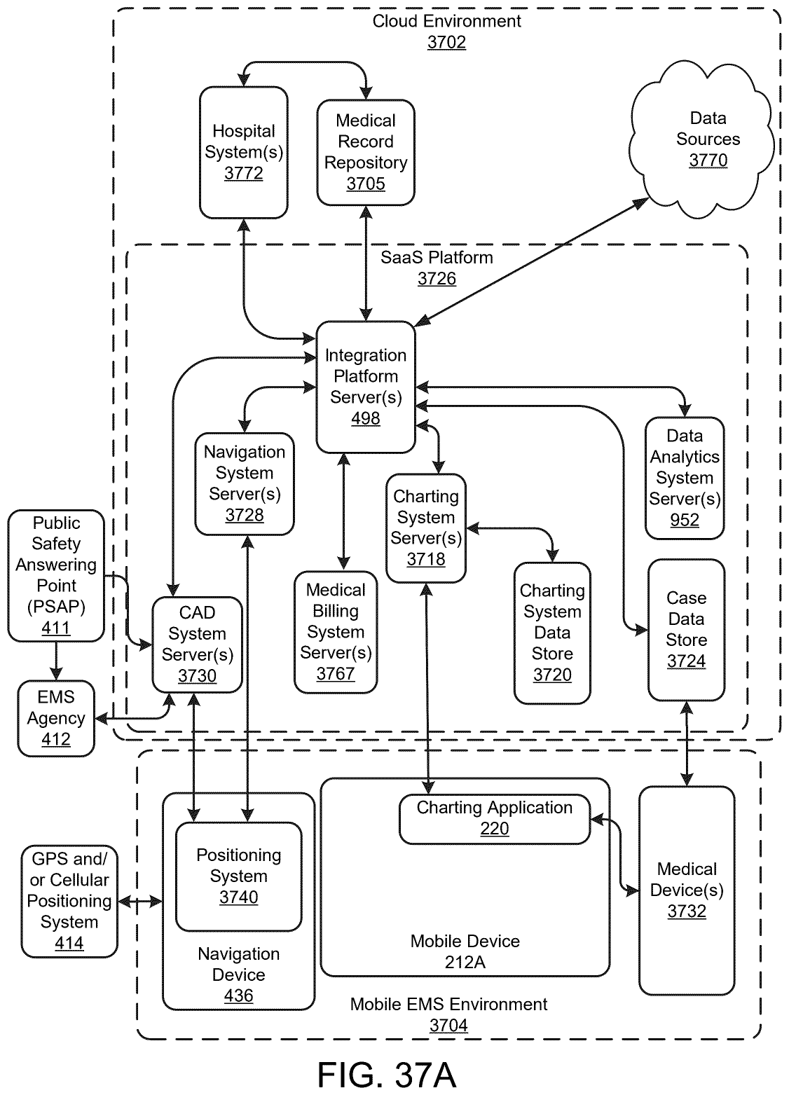

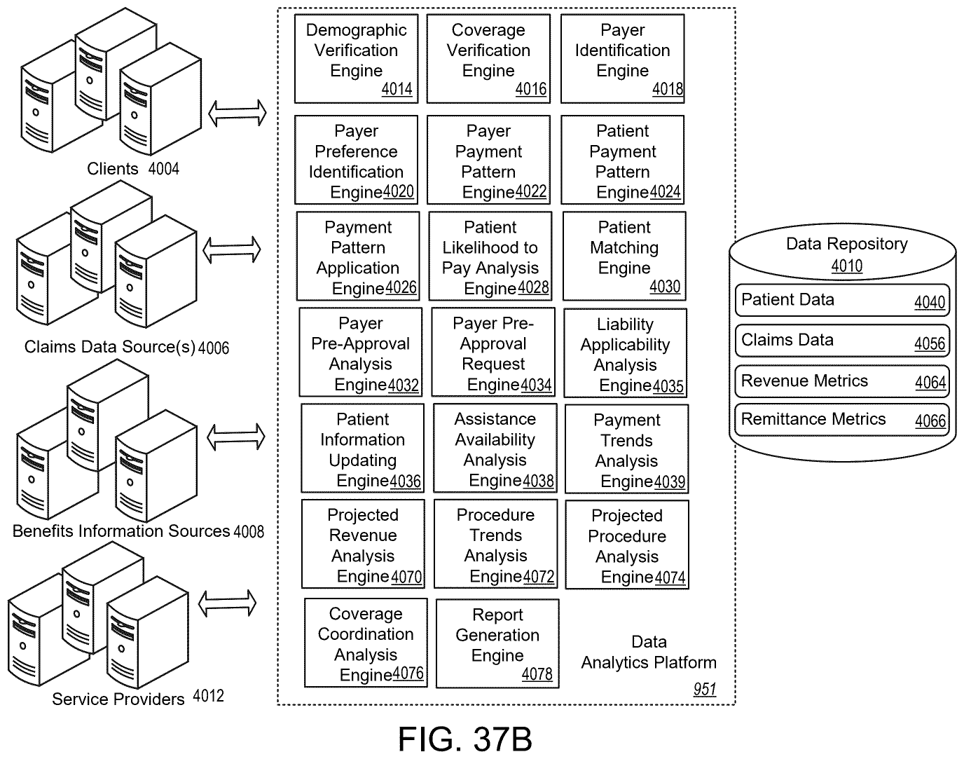

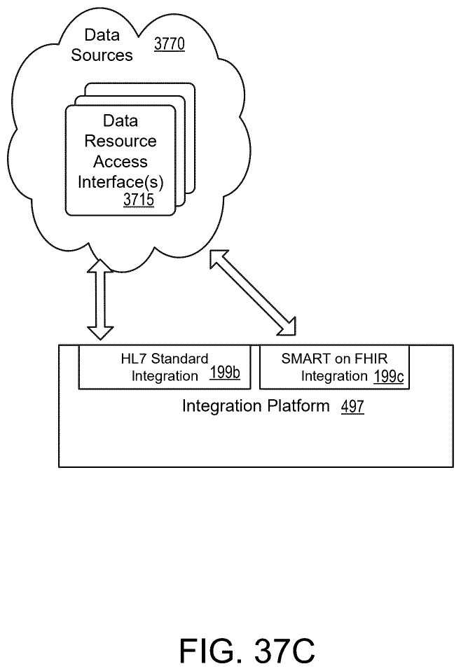

As shown in FIG. 2A, the clouds server(s) 226 can include one or more physical and/or virtual servers. The mobile computing devices 212A-212N can include any of a variety of portable computing platforms such as smartphones and/or tablet computers. Particular examples of the mobile computing devices 212A-212N are described further below with reference to FIG. 35. The medical device(s) 214 can include one or more of several types of medical devices, such as the medical devices described further below with reference to FIG. 37. For instance, the medical device(s) 214 can include a public access automated external defibrillator (AED), a professional defibrillator/patient monitor, a ventilator, a drug delivery device, a trauma kit, a mechanical compression device, etc. The public access AED may lack the capability to monitor physiological parameters such as pulse oximetery and capnography. In contrast, the professional defibrillator/patient monitor may include the capability to monitor physiological parameters such as pulse oximetry and capnography. Regardless of its particular configuration, as shown in FIG. 2A, the medical device(s) 214 may be configured to communicatively couple to the mobile computing device(s) 212A-212N to provide medical case file data to the ePCR during monitoring and/or treatment of the patient 210. In some examples, the medical device(s) 214 begin recording events upon power-up and, therefore, the ePCR data may include data generated before, during, and/or after the EMS caregivers 208A-208N monitor and/or treat the patient 210 with the medical device(s) 214. In an implementation, the medical device(s) 214 may provide data to the mobile EMS charting application 220 in real-time as the data is collected by the medical device, at various intervals during data collection, and/or at the conclusion of data collection from the patient by the medical device(s) 214. In an implementation, the medical device(s) 214 may be coupled to the wide area network 206 and may provide medical device data to the charting data store 228. The cloud EMS charting application 232 may retrieve the medical device data from the charting data store 228. The cloud EMS charting application 232 may append the medical device data for the encounter and/or may enter all or a portion of the medical device data into the ePCR created for the encounter.

In some examples illustrated by FIG. 2A, the medical device(s) 214 and the mobile computing devices 212A-212N include network interfaces configured to communicate with the network 218 via one or more standards supported by the network 218. Alternatively or additionally, in some examples, the network interfaces are configured to communicate directly via proximal connections. These proximal connections can be implemented via any of a variety of wired and/or wireless standards, such as Universal Serial Bus (USB), RFID, BLUETOOTH and/or NFC standards, among others. Additionally, the mobile computing devices 212A-212N may form a mesh network that connects with the network 206 through a subset of the mobile computing devices 212A-212N. This mesh network, or the other networks described herein, may implement peer to peer communications schemes.

In some examples illustrated by FIG. 2A, the network 206 may include any communication network through which devices in the cloud environment 202 and the EMS call environment 204 can exchange data. In some examples, the network 206 includes and supports wireless network and/or wired connections. For instance, in these examples, the network 206 can support one or more networking standards such as GSM, CMDA, USB, BLUETOOTH, CAN, ZIGBEE, Wireless Ethernet, Ethernet, and Transmission Control Protocol (TCP)/Internet Protocol (IP), among others. In at least one example, the network 206 includes both private networks, such as local area networks, public networks, such as the Internet, and cellular networks. It should be noted that, in some examples, the network 206 includes one or more intermediate devices involved in the routing of packets from one endpoint to another. However, in other examples, the network 206 involves only two endpoint devices that each have a network connection directly with the other.

As shown in FIG. 2A, the EMS caregivers 208A-208N are distinct individuals, However, the examples disclosed herein are not limited to use by a particular number or arrangement of EMS caregivers. For instance, in at least one example, a single EMS caregiver monitors and/or treats the patient 210 using the medical device(s) 214 and documents the EMS call using a single mobile computing device. Thus, the examples disclosed herein are not limited to a particular configuration of EMS caregivers and/or patients.

In some examples, the EMS charting system includes a mobile server within the EMS call environment. FIG. 2B illustrates one such example, an EMS charting system 250. As shown in FIG. 2B, the EMS charting system 250 includes the cloud environment 202 of FIG. 2A and a EMS call environment 254 that includes the features of the EMS call environment 204 of FIG. 2A and a mobile server 256. The mobile server 256 includes a charting data store 260. In an implementation, the mobile server 256 may include a charting data synchronization service 258. In these examples, the synchronization service 258 is configured to interoperate (e.g., via a set of API calls) with other synchronization service instances (e.g., each synchronization service 222 and the synchronization service 230) to maintain a common view of the ePCR data stored within the EMS charting system 200 for all mobile EMS charting applications (e.g., each mobile EMS charting application 220) and the cloud EMS charting application 232. In some examples, when processing these API calls, the synchronization service 258 transmits or receives, and processes synchronization messages that specify each change made to ePCR data, a timestamp of the change, and an identifier of the originator of the change (e.g., an automated process, a user, or another entity). Example processes that the synchronization service 258 is programmed to execute in this configuration are described further below with reference to FIG. 34. The charting data store 260 is configured to store ePCR data for manipulation by the synchronization service 258.

It should be noted that the API exposed by the synchronization services 222, 230, and 258 can be implemented using a variety of interoperability standards and architectural styles. For instance, in one example, the API is a web services interface implemented using a representational state transfer (REST) architectural style. In this example, the API communications are encoded in Hypertext Transfer Protocol (HTTP) along with JavaScript Object Notation and/or extensible markup language. In some examples, portions of the HTTP communications may be encrypted to increase security. Alternatively or additionally, in some examples, the API is implemented as a .NET web API that responds to HTTP posts to particular URLs with data descriptive of ePCR data stored in the charting data store 228, the charting data store 260, and/or each charting data store 223. Alternatively or additionally, in some examples, the API is implemented using simple file transfer protocol commands and/or a proprietary application protocol accessible via a TCP socket. Thus, the API as described herein is not limited to a particular implementation.

In certain examples, the synchronization service 222 is configured to maintain the charting data stores 223 in local storage for use by the mobile EMS charting application 220. In these examples, the synchronization service 222 is also configured to expose and implement an API through which ePCR data stored in the charting data stores 223 can be accessed by other processes, such as the mobile EMS charting application 220 or synchronization services (e.g. the synchronization service 230 and/or the synchronization service 258). In these examples, the synchronization service 222 can incorporate a service worker (or some other monitoring process executing on a thread distinct from a thread executing the mobile EMS charting application 220) configured to monitor and recognize a network connectivity status between the host device and the network 206 and/or the network 218. When the connection to the network 206 is active, the synchronization service 222 exchanges synchronization messages with the synchronization service 230. Likewise, when the connection to the network 218 is active, the synchronization service 222 exchanges synchronization messages with the synchronization service 258. However, when the connection to either the network 206 or the network 218 is inactive, the synchronization service 222 cashes synchronization messages addressed to the synchronization service associated with the inactive network and subsequently transmits the synchronization messages when the connection to the inactive network resumes.

FIG. 2C illustrates a synchronization infrastructure 277 implemented within an EMS charting system in at least one example. As shown in FIG. 2C, the synchronization infrastructure 277 includes the synchronization services 222A, 222B, 230, and 258 described above with reference to FIG. 2B. As is further shown in FIG. 2C, each of the synchronization services 222A, 222B, 230, and 258 is configured to implement a respective message queue 265A, 265B, 269, and 271. The message queues 265A, 265B, 269, and 271 are configured to store synchronization messages generated by manipulation of ePCR data within the respective charting data stores 223A, 223B, 228, and 260.

In some examples illustrated by FIG. 2C, the synchronization services 222A, 222B, 230, and 258 are configured to communicate synchronization messages 273A-273E to one another to maintain a common view of the ePCR data underlying the synchronization system. For instance, in one example, each of the synchronization services 222A, 222B, 230, and 258 subscribes to ePCR data channels published by the other synchronization services. Through these ePCR data channels, each synchronization service receives publications detailing changes made to charting data stores local to the other synchronization services. For instance, the synchronization service 222A may subscribe to ePCR data channels published by the synchronization services 222B, 230, and 258 if each of those services is implemented and available within the EMS charting system. These channels include all ePCR data or may be restricted to particular types/categories of ePCR data (e.g., clinical information), as described further below. Likewise, the synchronization service 222B may subscribe to ePCR data channels published by the synchronization services 222A, 230, and 258 if each of those services is implemented and available within the EMS charting system. Such subscription may involve a registration process that includes authentication and authorization of the synchronization services to ensure secure transfer of ePCR data.

The synchronization infrastructure 277 illustrated in FIG. 2C offers several benefits. For example, the synchronization services 222A, 222B, and 230 shield the mobile EMS charting applications 220A, 220B, 220C, and the cloud EMS charting application 232 from network disruptions by isolating data exchange through the network from these applications. This shielding can help the mobile EMS charting applications 220A, 220B, 220C, and the cloud EMS charting application 232 to maintain normal interactions with users despite degraded or unavailable network connectivity. Moreover, given that synchronization messages can communicate small portions of ePCR data, even as small as a single data field value, this approach to data transfer is well suited to the EMS environment, where the number of synchronization messages over time is relatively low due to many of the sources of data being manual entry. The latency between entry of a value in a data field of an ePCR in, for example, the charting data store 223A to population of the value in a data field of the ePCR in, for example, the charting data store 223B can be minimal. Minimal latency facilitates concurrent use of multiple mobile EMS charting applications by multiple users to complete a single, cross-device ePCR. Moreover, this minimal latency enables rapid transfer of ePCR data from distinct ePCRs, where two or more devices from two or more distinct EMS teams are exchanging and visually rendering ePCR data generated by both EMS teams using the features described herein. Additionally, synchronization messages can include information regarding changes to ePCR data that in-bulk transfers (e.g., entire ePCRs) do not, such as an originator of the change and the timestamp of the change. This information can be used advantageously to apply adjudication schemes to conflicts that are based on the role of an originator and/or a timing of the change. In sum, the synchronization infrastructure 277 illustrated in FIG. 2C provides support for multiple users to be able to capture data into the same ePCR—regardless of various different states of the devices (online and offline)—and ensure the data is all captured iteratively, as it is received over time, and saved without any conflicts.

As further shown in FIG. 2C, the synchronization services 222A, 222B, 230, and 258 utilize the message queues 265A, 265B, 269, and 271 to store and forward the synchronization messages 273A-273E as is sometimes required due to changing conditions and participants involved in EMS calls. For instance, consider an example in which an initial first responder crew, which may be a basic life support (BLS) crew. In this example, a local dispatch center may receive a call reporting damage to a parking structure and, in response thereto, may contact an EMS agency or fire department closest to the scene to dispatch the first responder crew. The BLS crew, for example, may be a two-person crew consisting of an emergency medical responder (EMR) and an emergency medical technician (EMT) that arrives at the scene of the partially collapsed parking structure and hears several persons in distress both on the street and within the partially collapsed parking structure. The EMR may carry a smartphone hosting the mobile EMS charting application 220B. The EMT may carry a smartphone hosting the mobile EMS charting application 220A. The EMR is logged into an EMS system via the mobile EMS charting application 220B. The EMT is logged into the EMS system via the mobile EMS charting application 220A. The synchronization service 222A is subscribed to an ePCR data channel offered and published by the synchronization service 222B. The synchronization service 222B is subscribed to an ePCR data channel offered and published by the synchronization service 222A.

In this example, the EMR proceeds immediately to locate and assess the state of the person in the parking structure, while the EMT assesses the state of the people on the street. The EMR locates the person in the parking structure, interacts with the mobile EMS charting application 220B to open an ePCR, takes the person's (now patient's) name, and inputs ePCR data specifying that the patient appears alert but is suffering from slurred speech. The mobile EMS charting application 220B stores the ePCR data in the charting data store 223B. In some examples, this ePCR data is associated with (e.g., stored in) a first ePCR that, in turn, is associated with this individual patient. In these examples, this first ePCR will be separate and distinct from any other ePCR generated by the BLS crew for any other patient. Further, in this example, due to the current location of the EMR, the smartphone 212B does not have network connectivity. As such, the synchronization service 222B generates and stores synchronization messages specifying the ePCR data collected by the EMR and stores the synchronization messages in the message queue 265B. In this example, the synchronization service 222B also publishes the synchronization messages to its ePCR data channel, so that processes subscribed therein (either local or remote) can access the synchronization messages.

Meanwhile, the EMT makes her way to the parking structure. During her approach, her smartphone 212A comes within range of the smartphone 212B and receives a BLUETOOTH low energy advertisement from the smartphone 212B. The smartphones 212A and 212B establish a direct connection. The synchronization service 222A detects this connection, determines that new content (in the form of new synchronization messages) is available via the ePCR channel of the synchronization service 222B and retrieves the new content. Next, the synchronization service 222A updates the charting data store 223A with the ePCR data specified in the newly received synchronization messages.

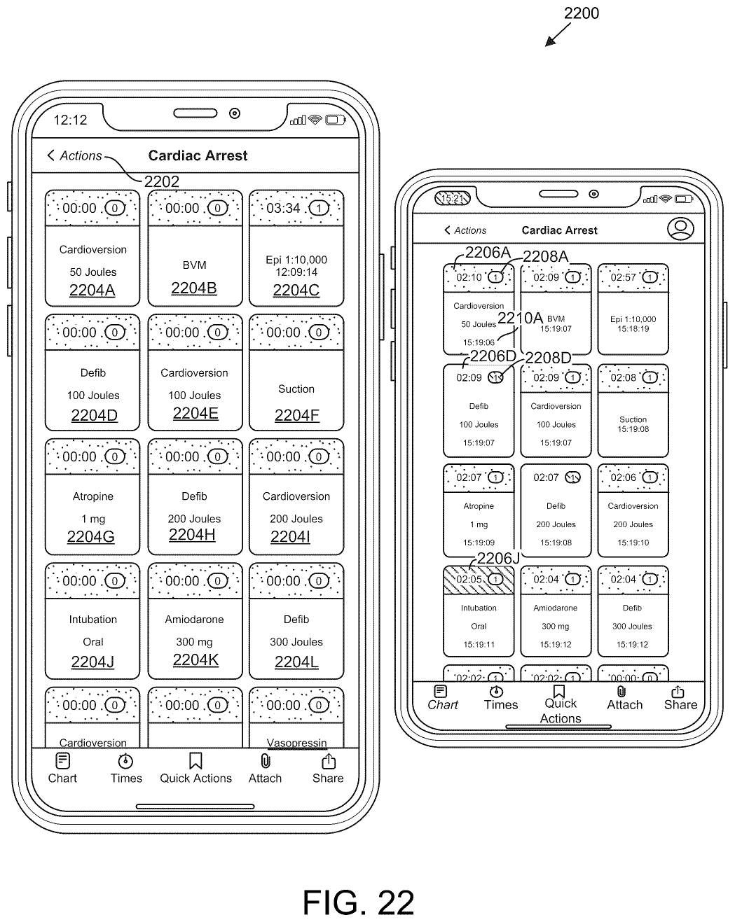

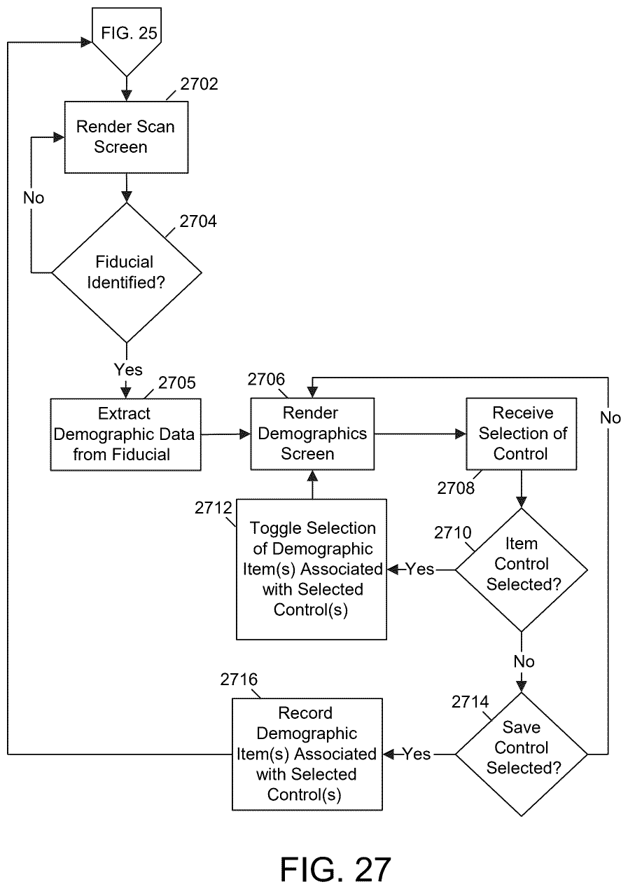

The EMT next assesses the patient and discovers that the EMR misunderstood the patient's name. The EMT opens the ePCR stored within the charting data store 223A by the synchronization service 222A via the mobile EMS charting application 220A and adjusts the patient's name in the ePCR data stored in the charting data store 223A. For example, the EMT may scan a driver's license or other patient identification documentation of the patient using features described with reference to FIG. 1E above and FIGS. 27-29 below. Further, in this example, the EMT determines that the patient is suffering from respiratory distress, administers oxygen to the patient, and documents the administration of the oxygen to the patient via a QA control. QA controls are described further below with reference to FIG. 22. The synchronization service 222A detects these changes and transmits synchronization messages to the synchronization service 222B.

The synchronization service 222B detects a conflict between the value of the patient's name stored in the charting data store 223B and the value of the patient's name specified in the newly received synchronization message. As such, the synchronization service 222B applies an arbitration process to the two values, identifies an arbitrated value of the patient's name and stores the arbitrated value as ePCR data in the charting data store 223B. Any of a variety of arbitration processes may be applied, as is discussed further below with reference to FIG. 34.

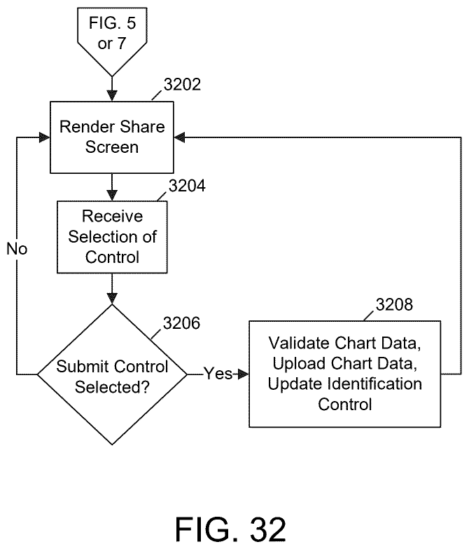

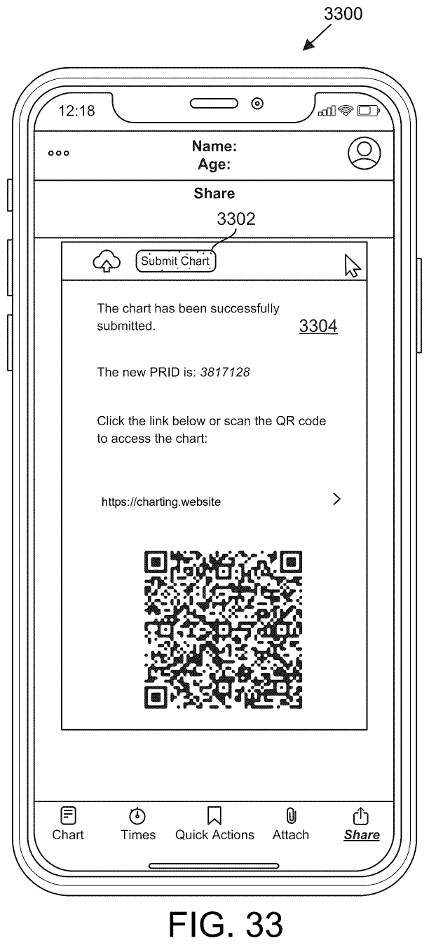

Continuing with this example, another crew arrives at the scene of the partially collapsed parking structure. This advanced life support (ALS) crew includes an advanced emergency medical technician (AEMT) and a paramedic. The ALS crew may be from, for example, a second EMS agency not affiliated with the first agency and located several miles away from the scene. Further, the ALS crew arrives in an ambulance that houses a mobile server (e.g., the mobile server 256 of FIG. 2B). The mobile server hosts the charting application 220C, the charting data store 260, and the synchronization service 258. The AEMT is logged into the EMS system via the mobile EMS charting application 220C. The ALS crew assumes care of the patient and interacts with the mobile EMS charting application 220C to generate a new ePCR and record ePCR data as interventions and other actions are taken. Afterward, the patient is loaded into the ambulance and transported to a nearby hospital for additional treatment. The transfer of patient care from the ALS crew to the hospital can be aided, in some examples, through the ePCR data upload features described further below with reference to FIGS. 32 and 33. It should be noted that, in some examples, the new ePCR generated by the mobile EMS charting application 220C through its interactions with the ALS crew is separate and distinct from any previous ePCR generated via interactions with the BLS crew, even where the new ePCR and a previous ePCR focus on the same patient. In other words, in these examples each ePCR documents, and is specific to, an individual encounter between a patient and one or more providers employed by a healthcare organization (e.g., is per patient and per agency). Moreover, each ePCR (e.g., each of the new ePCR and the previous ePCR) will be completed and validated by a respective agency and submitted for generation of a medical billing claim separately by its respective agency.

In this example, the synchronization services 258 and 230 are subscribed to one another's ePCR data channels. The ambulance also includes a mobile, directional antenna that provides the mobile server with a network connection to a high-speed wireless network (e.g., FirstNet commercially available from AT&T). The ambulance further includes a mobile WiFi router connected to the mobile server and in range of the smartphones 212A and 212B. The smartphones 212A and 212B connect to the mobile router. The smartphones 212A and 212B gain access, via the wireless network, to the trusted CAD system that originally dispatched the EMR and the EMT crew to this location. The CAD system communicates additional dispatch data to the smartphones 212A and 212B. This additional dispatch data specifies that the ambulance housing the mobile server was also dispatched to this location and that the mobile server hosts the trusted synchronization service 258. The additional dispatch data can include, for example, one or more reference codes that identify one or more patients at the scene, one or more identifiers of the call, and/or one or more identifiers of equipment on scene or dispatched to the scene (e.g., internet protocol addresses, service set identifiers, etc.), among other data. In response to receiving the additional dispatch data, the smartphones 212A and 212B subscribe to an ePCR data channel published by the synchronization service 258. The synchronization service 258, being in possession of the same dispatch data, subscribes to ePCR data channels published by the synchronization services 222A and 222B. Upon completion of these subscription processes, the synchronization services 222A, 222B, 230, and 258 are connected to one another and can exchange synchronization messages to maintain the charting data stores 228, 260, 223A and 223B. It should be noted that, if the high-speed wireless network connection is lost, the local WiFi connections may continue to be utilized by the mobile server and the smartphones 212A and 212B to communicate ePCR data via the publication-subscription processes described herein. Moreover, in some examples, should the high-speed wireless network connection be restored, published synchronization messages queued by, for example, the synchronization service 258 may be retrieved and processed by the synchronization service 230.

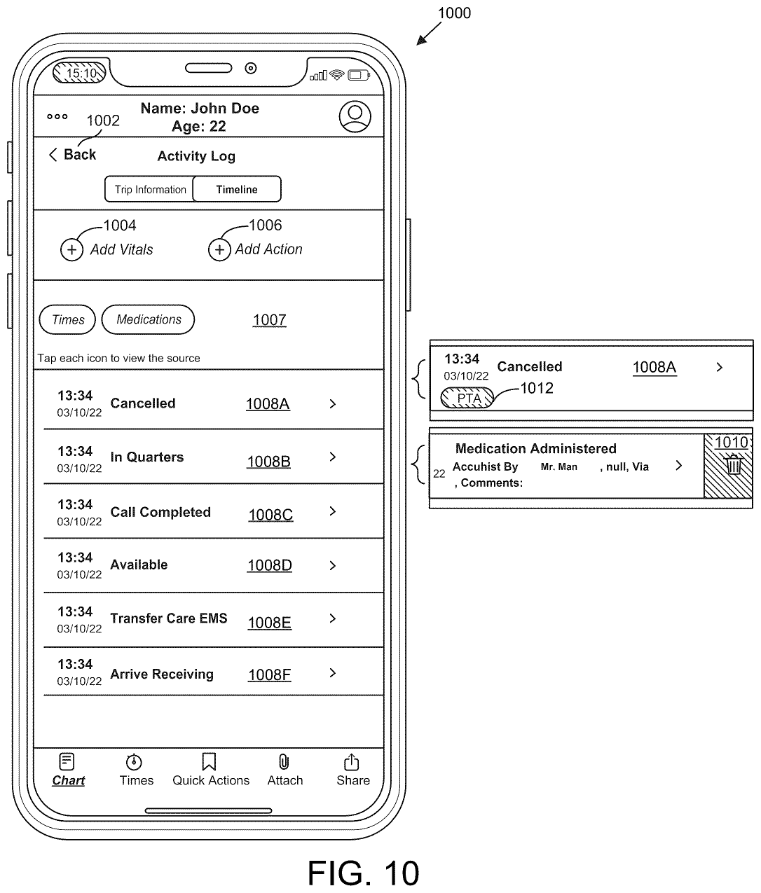

In some implementations, the charting applications 220A and 220B are configured to interact with the EMR and the EMT to authorize the subscription processes described above. For instance, in some examples, the charting applications 220A and 220B are each configured to respond to the additional dispatch data described above (as received from the CAD system or via any other device described herein) by rendering, via a user interface of their host device, a prompt requesting authorization to share the ePCR data generated by the BLS crew for a particular patient with the ALS crew for that same particular patient. In these examples, the charting applications 220A and 220B are each configured to receive user input specifying whether authorization is granted and, if the user input specifies that authorization is granted, allow the synchronization service 258 to subscribe to the synchronization services 222A and 222B. Further, in these examples, the charting applications 220A and 220B are each configured to prevent the synchronization service 258 from subscribing to the synchronization services 222A and 222B if the user input specifies that authorization is denied. This authorization or denial controls whether the ePCR data generated by the BLS crew is published to the synchronization service 258 and, in turn, stored in the charting data store 260 and made available to the ALS crew via the charting application 220C. In some examples, if the ePCR data generated by the BLS crew is made available to the ALS crew via the charting application 220C, the charting application 220C labels or otherwise indicates that the ePCR data originated from the BLS crew. An example of one approach to indicating the source of ePCR data from a first ePCR within a visual representation of a second ePCR is described further below with reference to FIG. 10. In these examples, for a more complete patient treatment and health history record the second ePCR may incorporate data from the first ePCR and the visual representation of the second ePCR includes ePCR data from the first ePCR. However, the data associated with each of the two ePCRs is identifiable and each ePCR provides a separately billable record. In this manner, each agency or crew can track and bill for treatments that they provided in an encounter with a patient but the patient treatment is enhanced by enabling each agency or crew to view actions from another agency or crew within their own record. The caregivers are relieved of the burden of having to open multiple records and/or view records on separate devices in order to provide efficient medical care that retains some continuity even with crew transitions. It should be noted that the inclusion of ePCR data from a first ePCR within the visual representation of a second ePCR, in the system described herein, is different from visual representations rendered by other ePCR systems, which include only ePCR data from a single ePCR.

In some implementations, the charting application 220C is configured to interact with the AEMT to authorize the subscription processes described above. For instance, in some examples, the charting application 220C is configured to respond to dispatch data indicating other instances of the charting application 220 (e.g., the charting applications 220A and 220B) are on scene by rendering, via a user interface of its host device, a prompt requesting authorization to accept the ePCR data generated by the other instances (e.g., the BLS crew). In these examples, the charting application 220C is configured to receive user input specifying whether authorization is granted and, if the user input specifies that authorization is granted, allow the synchronization service 258 to subscribe to the synchronization services 222A and 222B. Further, in these examples, the charting application 220C is configured to prevent the synchronization service 258 from subscribing to the synchronization services 222A and 222B if the user input specifies that authorization is denied. This authorization or denial controls whether the ePCR data generated by the BLS crew is published to the synchronization service 258 and, in turn, stored in the charting data store 260 and made available to the ALS crew via the charting application 220C. In some examples, the ePCR data that is generated by the BLS crew and stored in a first ePCR prior to the arrival (PTA) of the ALS crew is flagged as such when reviewed by ALS crew members within a visual representation of a second ePCR being completed by the ALS crew. One example of a prior-to-arrival (PTA) control that the charting application 220C is configured to render when displaying ePCR data generated by a crew that arrived at a scene prior to the viewer is illustrated and described further below with reference to FIG. 10. It should be noted that, in some examples, the prompting, receiving of input, and grant or denial operations described above are executed on a per patient basis (e.g., one prompt per patient). In other examples, these operations are executed on groups of patients to speed the sharing of ePCR data associated with the patients among onsite EMS personnel.

Implementations that require authorization of subscription processes can be useful where, as described above, two or more crews are not part of affiliated organizations but use instances of the charting application 220. For instance, in the example described above, the charting applications 220A, 220B, and 220C are part of a single EMS charting system, but the charting application 220A and 220B are associated with a first tenant and the charting application 220C is associated with a second tenant. In other examples, the charting applications 220A and 220B are associated with a first EMS charting system that is physically and logically distinct from a second EMS charting system that is associated with the charting application 220C. In these other examples, the devices that host the charting applications 220A, 220B, and 220C may communicate with one another via a common network (e.g., the local network 218 described above with reference to FIGS. 2A and 2B) but the charting applications 220A and 220B may be configured to communicate with a first cloud environment (e.g., the cloud environment 202) that is separate and distinct from a second cloud environment with which the charting application 220C is configured to communicate.

It should be noted that making data available from the first ePCR, generated, for example, by the BLS team, within the charting application at the second device providing the second ePCR, generated, for example, by the ALS team does not merely concatenate the two ePCR records into one single comprehensive ePCR record. As an initial consideration, the information from the first ePCR may be limited to information relevant to the care provided by the ALS team. Thus the information transfer may be selective and limited to clinical information such as medical history, time stamps of interventions, types of interventions, allergy information, medications administered, etc. Information from the first ePCR related to the crew or the response such as crew identification, time of arrival, delays in arrival, narratives, etc. may be excluded. As a further consideration, each ePCR provides a self-contained and complete record of the entire response provided by the associated team for legal liability, billing, quality control, quality improvement, agency records, etc. Additionally, crews that respond to a patient call may originate from EMS agencies that are completely unrelated to one another other than the fact that they happened to each send a crew to a same scene. The methods of storing data and formatting data along with agency requirements for the type and frequency of data collection, the protocols provided, the particular interventions and/or medication provided, the names of medications, agency vernacular, agency procedures, etc. may be different for each agency and thus different for the respective ePCRs. The system described herein enables interoperability between these disparate systems. Moreover, it should be noted that once crews from distinct agencies have agreed to share ePCR data, the system described herein communicates ePCR data of the type/category agreed to in real time or near real time. As such, in some examples, ePCR data entered into a first ePCR by a first crew is communicated automatically to a device of a second crew as the ePCR data is entered, so that the ePCR data can be rendered or otherwise provided in conjunction with ePCR data entered by the second crew into a second ePCR).

Thus, as can be understood in view of the discussion above, features of the EMS charting system enable seamless transition of patient care between care providers at least in some implementations. For example, the transitions may be from a BLS crew to an ALS crew, from EMS (BLS or ALS) to a hospital, rehabilitation center, or pre-scheduled medical service provider (e.g., for dialysis, chemotherapy, infusions, etc.), from a fire department crew to EMS (BLS or ALS), from a ground transportation EMS crew to an air transportation crew, etc.

### EMS Charting Screens and Processes

FIGS. 3, 5, 7, 9, 11, 13, 15, 17, 19, 21, 23, 25, 27, 30, and 32 collectively illustrate a process 300 for generating an ePCR to document an EMS call involving a patient (e.g., the patient 210 of FIG. 2A) and one or more EMS caregivers (e.g., the EMS caregivers 208A-208N of FIG. 2A).

As shown in FIG. 3, the ePCR generation process 300 starts with the mobile EMS charting application controlling a device (e.g., one of the mobile computing devices 212A-212N) that hosts the mobile EMS charting application to render 302 a chart selection screen within a user interface of the host device. FIG. 4 illustrates one example of a chart selection screen 400 that may be rendered 302 in some examples. As shown in FIG. 4, the chart selection screen 400 includes an add control 402, a filter control 404, and existing chart controls 406A-406E. As shown in FIG. 4, each of the existing chart controls 406A-406E can be altered by the mobile EMS charting application to include a more control 408 and a delete control 410.

In some examples, the user (e.g., one of the EMS caregivers 208A-208N of FIG. 2A) can select the new chart control 402 to start a new ePCR to document a service call. Alternatively, the user can select one of the existing chart controls 406A-406E to perform additional processing of a previously started, but incomplete or open (e.g., by another user via another device), ePCR. This additional processing can include revision of data fields stored in the ePCR, adding new values to data fields of the ePCR, and/or uploading the ePCR to a remote server (e.g., the cloud server(s) 226 of FIG. 2A and/or the mobile server 256 of FIG. 2B). Further details regarding the additional processing available via the existing chart controls 406A-406E are described further below. In some examples, the user can select one or more values (e.g., “Incomplete”, “Complete”, “Needs Review”) displayed within the filter control 404 to limit the existing chart controls 406A-406E displayed to those associated with an ePCR storing, or being associated with, the value. Further, the user can swipe one of the existing chart controls 406A-406E to reveal the more control 408 and the delete control 410. The more control 408 can be used to edit the ePCR. The delete control 410 can be used to delete the ePCR.

Returning to the process 300 with reference to FIG. 4, the mobile EMS charting application receives 304 input selecting a control. The mobile EMS charting application determines 306 whether a value within the filter control 404 was selected. If a value within the filter control 404 was selected, the mobile EMS charting application toggles selection of the value and filters 308 the one or more existing chart controls 406A-406E to those associated with ePCRs storing, or being associated with, the currently selected value(s) within the filter control 404. Subsequent to the operation 308, the mobile EMS charting application returns to operation 302. If no value within the filter control 404 was selected, the mobile EMS charting application proceeds to operation 310.

Continuing with the process 300, the mobile EMS charting application determines 310 whether the add control 402 was selected. If the add control 402 was selected, the mobile EMS charting application proceeds to operation 502 illustrated in FIG. 5. If the add control 402 was not selected, the mobile EMS charting application proceeds to operation 312.

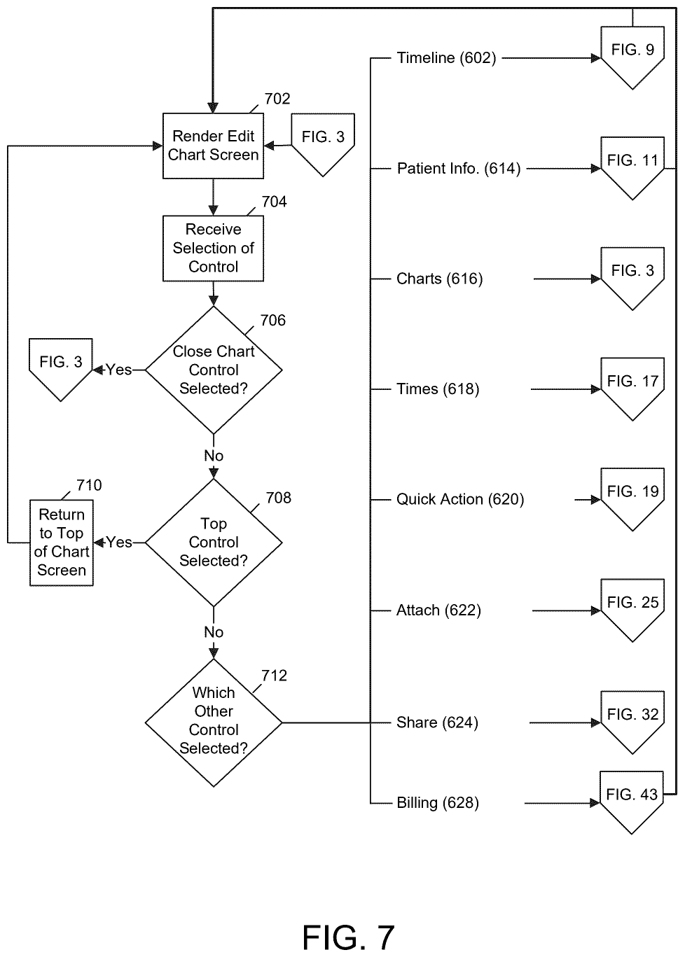

Continuing with the process 300, the mobile EMS charting application determines 312 whether one of the existing chart controls 406A-406E was selected. If one of the existing chart controls 406A-406E was selected, the mobile EMS charting application proceeds to operation 702 illustrated in FIG. 7. If none of the existing chart controls 406A-406E was selected, the mobile EMS charting application proceeds to operation 314.

Continuing with the process 300, the mobile EMS charting application determines 314 whether one of the existing chart controls 406A-406E was swiped. If one of the existing chart controls 406A-406E was swiped, the mobile EMS charting application alters 316 the swiped control to include a more control 408 and a delete control 410 and proceeds to operation 318. If none of the existing chart controls 406A-406E was swiped, the mobile EMS charting application executes 326 a process associated with the selected control. For instance, if one of the controls in the navigation bar was selected, the mobile EMS charting application controls the host device to render a screen associated with the selected control.

Continuing with the process 300, the mobile EMS charting application receives 318 input selecting a control. The mobile EMS charting application determines 320 whether the more control 408 was selected. If the more control 408 was selected, the mobile EMS charting application may proceed to operation 702 illustrated in FIG. 7. If the more control 408 was not selected, the mobile EMS charting application determines 322 whether the delete control 410 was selected. If the delete control 410 was selected, the mobile EMS charting application deletes 324 the ePCR associated with the delete control 410 from ePCR data in the local data store and proceeds to the operation 302. If the delete control 410 was not selected, the mobile EMS charting application proceeds to the operation 306.

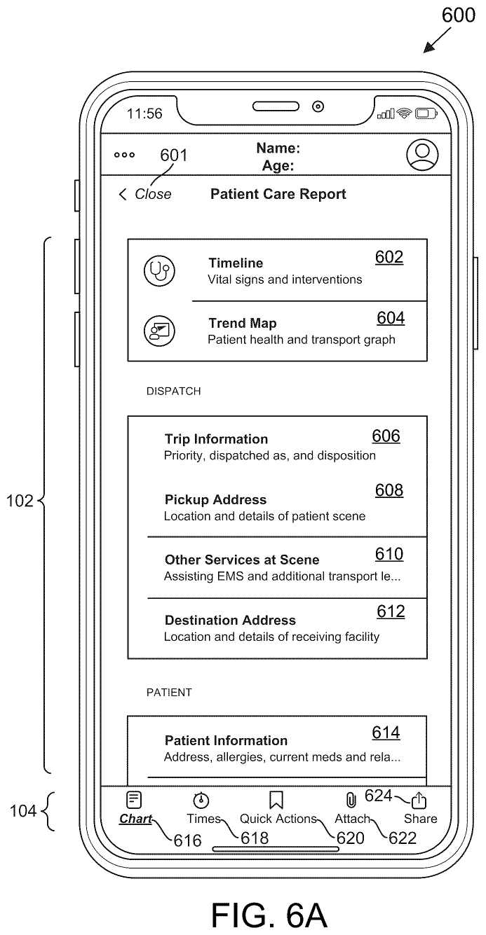

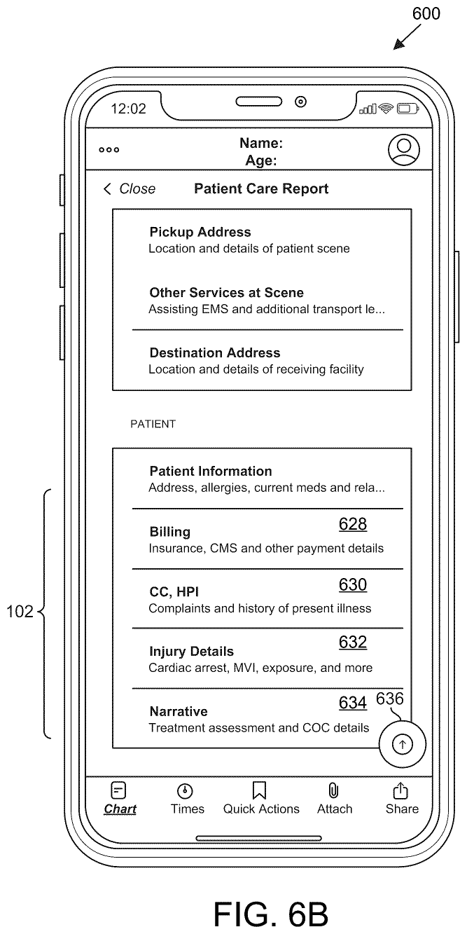

Continuing with the process 300 with reference to FIG. 5, the mobile EMS charting application controls the host device to render 502 a new chart screen within the user interface of the host device. FIGS. 6A and 6B illustrate one example of a new chart screen 600 that may be rendered 502 in some examples. As shown in FIGS. 6A and 6B, the new chart screen 600 includes a close control 601, the section controls 102 of FIG. 1, and the navigation bar 104 of FIG. 1. The section controls 102 include a timeline control 602, a trend map 604, a trip control 606, a pickup address control 608, a services control 610, a destination control 612, a patient information control 614, a billing control 628, a complaint control 630, an injury control 632, a narrative control 634, and a top control 636. The navigation bar includes a chart control 616, a times control 618, a QA control 620, an attach control 622, and a share control 624.

In some examples, the user can select the close control 601 to close the current ePCR and navigate back to the chart selection screen of FIG. 4. The user can select the timeline control 602 to navigate to a timeline screen useful to maintain ePCR data that documents activities performed by the user. One example of a timeline screen 1000 is illustrated and further described with reference to FIG. 10. The user can select the trend map control 604 to navigate to a trends screen that shows graphical representations of dynamic variables, such as patient physiologic parameters.

In some examples, the user can select the trip control 606 to navigate to a trip information screen useful to maintain ePCR data that documents attributes of travel to the EMS call location. One example of a trip information screen (e.g., the trip information screen 138) is illustrated and further described with reference to FIG. 1D. The user can select the pickup address control 608 to navigate to a map screen that displays the EMS call location and information descriptive of the same. In some examples, the pickup address information presented by the map screen is received directly from a CAD system, and the mobile EMS charting application is configured to determine a route between the current location of the user and the pickup address via global positioning system (GPS) services available within the host device. In these examples, the cloud EMS charting application is also configured to display the route in the map screen. The user can select the services control 610 to navigate to a services screen that displays information regarding other EMS caregivers and equipment dispatched to the EMS call. In some examples, the service information presented by the service control 610 is received directly from the CAD system. The user can select the destination address control 612 to navigate to a map screen that displays a receiving facilities location and information descriptive of the same. In some examples, the destination address information presented by the pickup address control 608 is received directly from a CAD system, and the mobile EMS charting application is configured to determine a route between the current location of the user and the destination address via GPS services available within the host device. In these examples, the cloud EMS charting application is also configured to display the route in the map screen.

In some examples, the user can select the patient information control 614 to navigate to a patient information screen useful to maintain ePCR data that documents information regarding the patient. One example of a patient information screen 1200 is illustrated and further described with reference to FIG. 12. The user can select the billing control 628 to navigate to a billing screen useful to maintain ePCR data that documents billing information regarding the patient. In some examples, the mobile EMS charting application is configured to import this data from a billing system. The user can select the complaint control 630 to navigate to a complaint screen useful to maintain ePCR data that documents information regarding the chief complaint and the history of the present illness of the patient. The user can select the injury control 632 to navigate to an injury screen useful to maintain ePCR data that documents information regarding an injury suffered by the patient. The user can select the narrative control 634 to navigate to a narrative screen useful to maintain ePCR data that documents narrative text regarding patient assessment and treatment. The user can select the top control 636 to scroll quickly to the top of the screen 600.

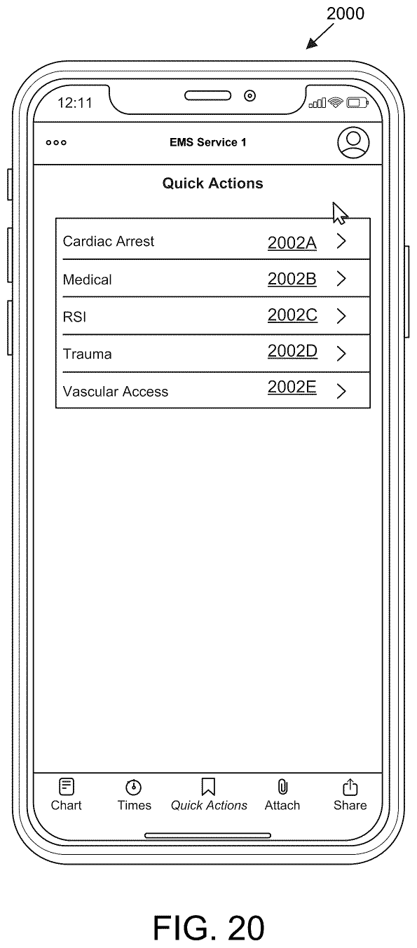

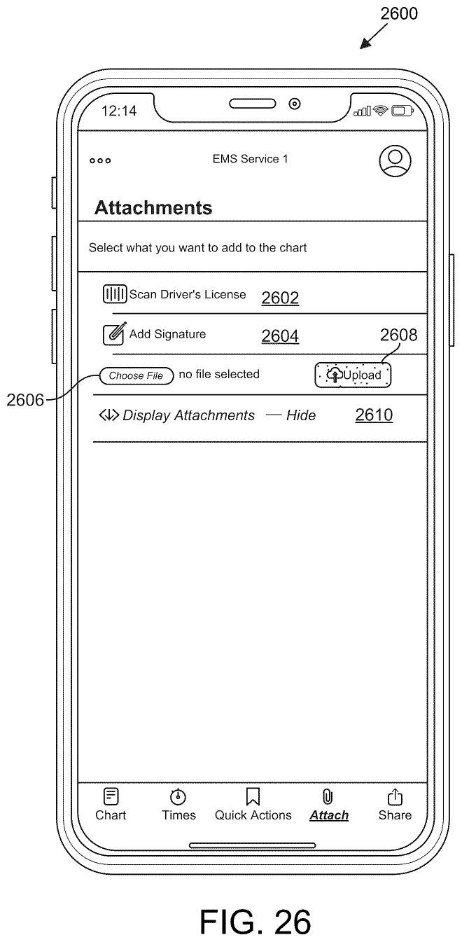

In some examples, the user can select the chart control 616 to navigate quickly to a chart selection screen useful to open a new ePCR or reopen an existing ePCR. One example of a chart selection screen (e.g., the chart selection screen 400) is illustrated and further described with reference to FIG. 4. The user can select the times control 618 to navigate quickly to a times entry screen useful to maintain ePCR data that documents the date and time of events related to the EMS call. One example of a times entry screen (e.g., the times entry screen 150) is illustrated and further described with reference to FIG. 1D. The user can select the QAs control 620 to navigate quickly to a QAs selection screen useful to further navigate to a QA screen. One example of a QAs screen 2000 is illustrated and further described with reference to FIG. 20. The user can select the attach control 622 to navigate quickly to an attachments screen useful to maintain ePCR data that documents signatures and imported patient demographic data. One example of an attachments screen 2600 is illustrated and further described with reference to FIG. 26. The user can select the share control 624 to navigate quickly to a share screen useful to upload completed ePCRs to a cloud EMS charting application (e.g., the cloud EMS charting application 232 of FIG. 2A). One example of a share screen 3300 is illustrated and further described with reference to FIG. 33.

Returning to the process 300 with combined reference to FIGS. 5 and 6, the mobile EMS charting application receives 504 input selecting a control. The mobile EMS charting application determines 506 whether the close control 601 was selected. If the close control 601 was selected, the mobile EMS charting application proceeds to operation 302 of FIG. 3. If the close control 601 was not selected, the mobile EMS charting application proceeds to operation 508.

Continuing with the process 300, the mobile EMS charting application determines 508 whether the top control 636 was selected. If the top control 636 was selected, the mobile EMS charting application controls the host device to scroll 510 to the top of the screen 600 and proceeds to the operation 504. If the top control 636 was not selected, the mobile EMS charting application proceeds to operation 512.

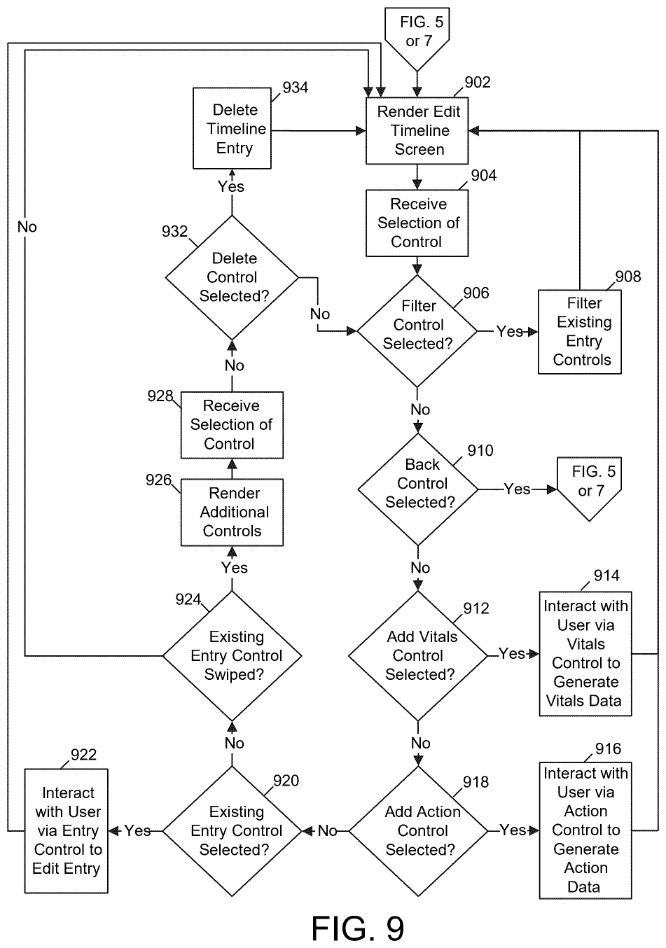

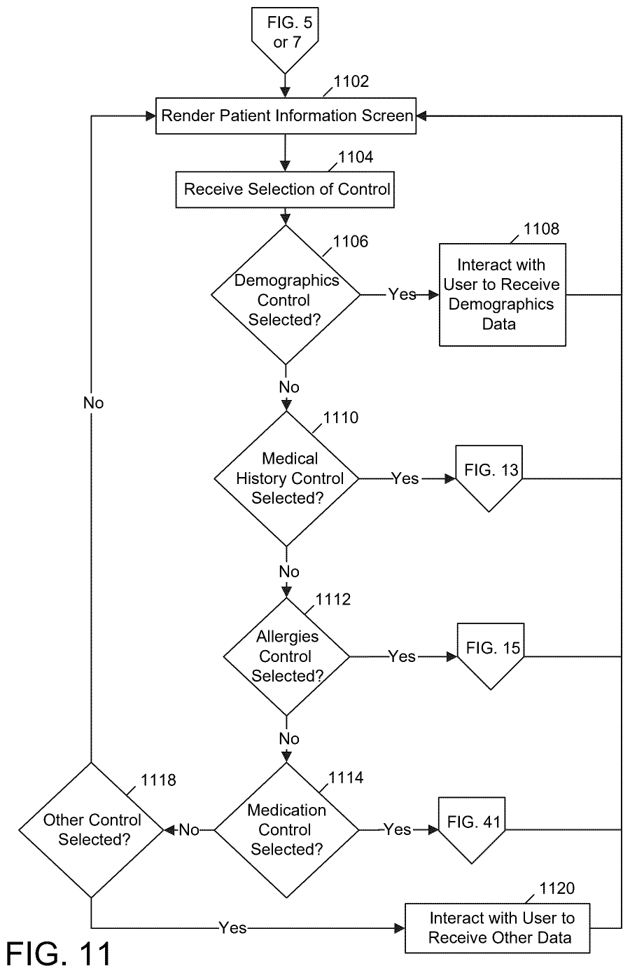

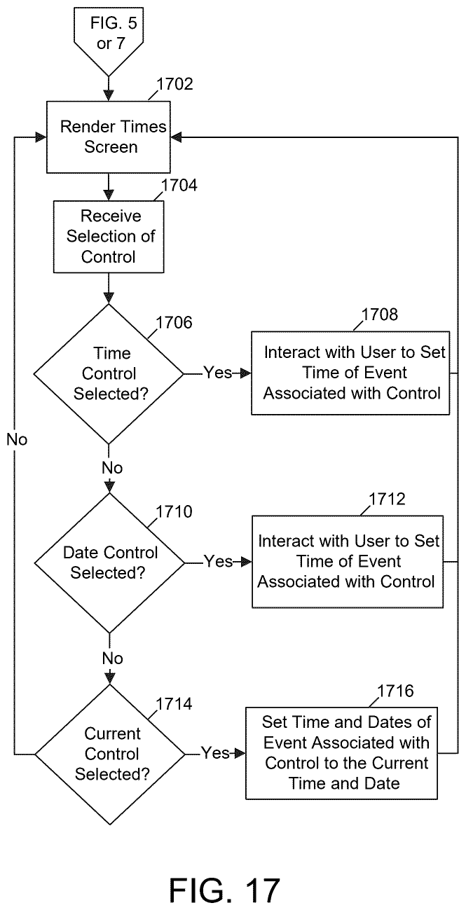

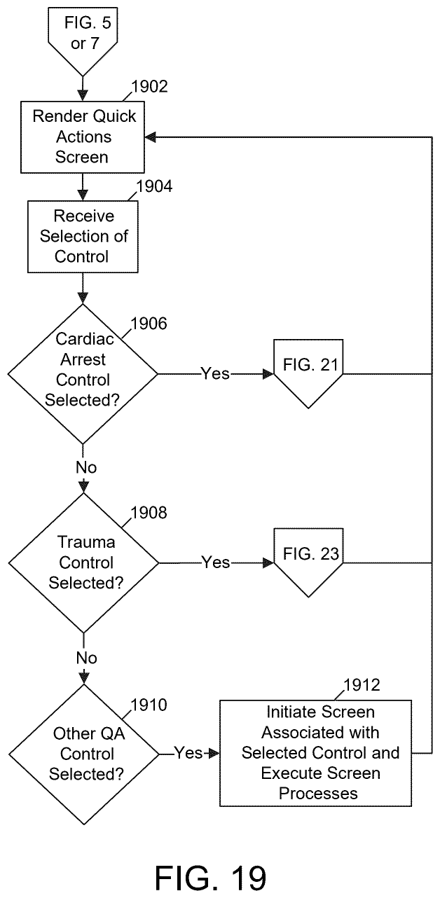

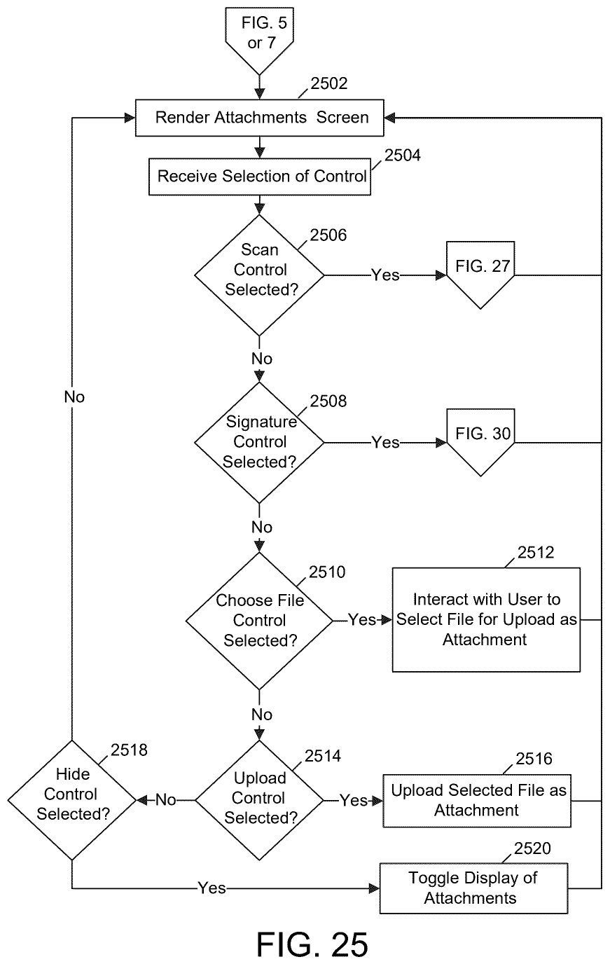

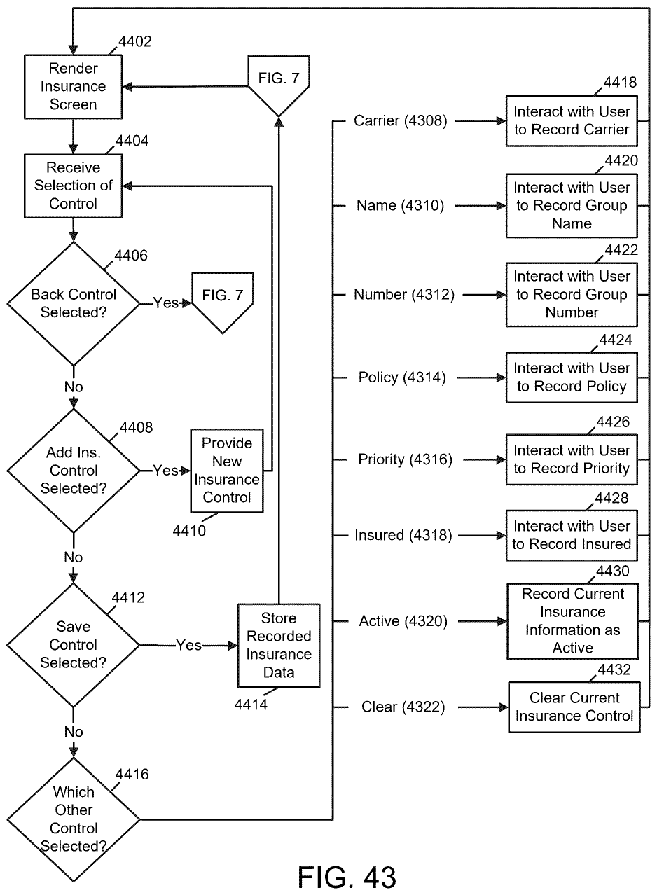

Continuing with the process 300, the mobile EMS charting application determines 512 which other control was selected. If the timeline control 602 was selected, the mobile EMS charting application proceeds to operation 902 illustrated in FIG. 9. If the patient information control 614 was selected, the mobile EMS charting application proceeds to operation 1102 illustrated in FIG. 11. If the chart control 616 was selected, the mobile EMS charting application proceeds to operation 302 illustrated in FIG. 3. If the times control 618 was selected, the mobile EMS charting application proceeds to operation 1702 illustrated in FIG. 17. If the QA control 620 was selected, the mobile EMS charting application proceeds to operation 1902 illustrated in FIG. 19. If the attach control 622 was selected, the mobile EMS charting application proceeds to operation 2502 illustrated in FIG. 25. If the share control 624 was selected, the mobile EMS charting application proceeds to operation 3202 illustrated in FIG. 32. If the billing control 628 was selected, the mobile EMS charting application proceeds to operation 4402 illustrated in FIG. 43.

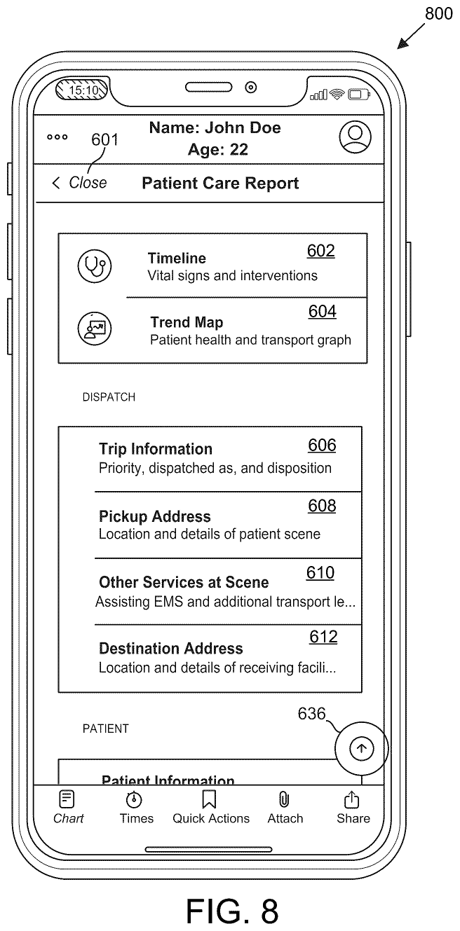

Continuing with the process 300 with reference to FIG. 7, the mobile EMS charting application controls the host device to render 702 a chart edit screen within the user interface of the host device. FIG. 8 illustrates one example of a chart edit screen 800 that may be rendered 702 in some examples. The chart edit screen 800 includes the same controls as the new chart screen 600 described above with reference to FIGS. 6A and 6B. As such, descriptions of these controls will not be repeated here, but it should be noted that the mobile EMS charting application operates on a pre-existing ePCR via the screen 800, rather than creating and operating on a new ePCR as is the case with regard to the screen 600.

Continuing with the process 300, the remainder of the operations illustrated in FIG. 7 (i.e., operation 704-712) are executed as do corresponding operations illustrated in FIG. 5 (i.e., 504-512). As such, descriptions of these operations will not be repeated here, but it should be noted that the mobile EMS charting application operates on a pre-existing ePCR via the operations illustrated in FIG. 7, rather than operating on a new ePCR as is the case with regard to the operations illustrated in FIG. 5.

Continuing with the process 300 with reference to FIG. 9, the mobile EMS charting application controls the host device to render 902 a timeline edit screen within the user interface of the host device. FIG. 10 illustrates one example of a timeline edit screen 1000 that may be rendered 902 in some examples. In some examples, the mobile EMS charting application puts entries in chronological order based on the timestamps created at data entry. As shown in FIG. 10, the timeline edit screen 600 includes a back control 1002, an add vitals control 1004, an add action control 1006, a filter control 1007, and existing entry controls 1008A-1008F. As shown in FIG. 10, each of the existing entry controls 1008A-1008F can be altered by the mobile EMS charting application to include a delete control 1010 or to include a PTA control 1012 that indicates the existing entry control reflects ePCR data generated prior to arrival of the device hosting the mobile EMS charting application. For instance, in some examples, the mobile EMS charting application is configured to read, from local memory, a timestamp that specifies the arrival time of the user or the user's crew at the scene of a patient who is the subject of the ePCR. In these examples, the mobile EMS charting application is further configured to include the PTA control 1012 within any existing entry controls 1008A-1008F that are associated with timestamps that predate the arrival timestamp. Such timeline entries may be created, for example, within a distinct ePCR by another instance of the mobile EMS charting application under the control of another user associated with a different crew and made available to this instance of the mobile EMS charting application via an authorized subscription process. In this way, the mobile EMS charting application enables a subsequent user to see interventions and other activities performed by an earlier crew within the context of the overall encounter timeline.

In some examples, the user can select the back control 1002 to navigate to the previously displayed screen. The user can select the add vitals control 1004 to access a screen configured to receive patient vitals and to add ePCR data to a local data store that specifies the patient vitals. The user can select the add action control 1006 to access a screen configured to receive information descriptive of an action of the user and to add ePCR data to a local data store that specifies the caregiver action. The user can select one or more entry types (e.g., “Times” or “Medications”) displayed within the filter control 1007 to limit the existing entry controls 1008A-1008F to those entries of the selected type. The user can select one of the existing entry controls 1008A-1008F to review and modify the ePCR data field(s) specified by the entry. Further, the user can swipe one of the existing entry controls 1008A-1008F to reveal the delete control 1010 and select the delete control 1010 to delete the entry associated with the swiped control.

Returning to the process 300 with combined reference to FIGS. 9 and 10, the mobile EMS charting application receives 904 input selecting a control. The mobile EMS charting application determines 906 whether an entry type within the filter control 1007 was selected. If a type within the filter control 1007 was selected, the mobile EMS charting application toggles selection of the type and filters 908 the one or more existing entry controls 1008A-1008F to those entries of the selected type. Subsequent to the operation 908, the mobile EMS charting application returns to operation 902. If no value within the filter control 1107 was selected, the mobile EMS charting application proceeds to operation 910.

Continuing with the process 300, the mobile EMS charting application determines 910 whether the back control 1002 was selected. If the back control 1002 was selected, the mobile EMS charting application proceeds to either operation 502 of FIG. 5 (e.g., if the EMS charting device proceeded to operation 902 from operation 512 of FIG. 5) or operation 702 of FIG. 7 (e.g., if the EMS charting device proceeded to operation 902 from operation 712 of FIG. 7). If the back control 1002 was not selected, the mobile EMS charting application proceeds to operation 912.

Continuing with the process 300, the mobile EMS charting application determines 912 whether the add vitals control 1004 was selected. If the add vitals control 1004 was selected, the mobile EMS charting application interacts 914 with the user to receive vitals data and stores, in a local data store, a timeline entry specifying the vitals data. The timeline entry specifies both the vitals data and a timestamp indicating when the vitals data was received. Subsequent to execution of the operation 914, the mobile EMS charting application proceeds to the operation 902. If the add vitals control 1004 was not selected, the mobile EMS charting application proceeds to operation 918.

Continuing with the process 300, the mobile EMS charting application determines 918 whether the add action control 1006 was selected. If the add action control 1006 was selected, the mobile EMS charting application interacts 916 with the user to receive action data and stores, in a local data store, a timeline entry specifying the action data (e.g., data specifying an intervention). The timeline entry specifies both the action data and a timestamp indicating when the action data was received. Subsequent to execution of the operation 916, the mobile EMS charting application proceeds to the operation 902. If the add action control 1006 was not selected, the mobile EMS charting application proceeds to operation 920.

Continuing with the process 300, the mobile EMS charting application determines 920 whether one of the existing entry controls 1008A-1008F was selected. If one of the existing entry controls 1008A-1008F was selected, the mobile EMS charting application interacts 922 with the user to edit the timeline entry associated with the selected entry control. Subsequent to execution of the operation 922, the mobile EMS charting application proceeds to the operation 902. If none of the existing entry controls 1008A-1008F was selected, the mobile EMS charting application proceeds to operation 924.

Continuing with the process 300, the mobile EMS charting application determines 924 whether one of the existing entry controls 1008A-1008F was swiped. If one of the existing entry controls 1008A-1008F was swiped, the mobile EMS charting application alters 926 the swiped control to include the delete control 1010 and proceeds to operation 928. If none of the existing entry controls 1008A-1008F was swiped, the mobile EMS charting application proceeds to operation 902.

Continuing with the process 300, the mobile EMS charting application receives 928 input selecting a control. The mobile EMS charting application determines 932 whether the delete control 1010 was selected. If the delete control 1010 was selected, the mobile EMS charting application deletes 934 the timeline entry associated with the delete control 1010 from the local data store and proceeds to the operation 902. If the delete control 1010 was not selected, the mobile EMS charting application proceeds to the operation 906.

It should be noted that, using the timeline screen 1000, the user can iteratively add entries to the timeline and thereby build a chronological list of actions taken within the EMS call.

Continuing with the process 300 with reference to FIG. 11, the mobile EMS charting application controls the host device to render 1102 a patient information screen within the user interface of the host device. FIG. 12 illustrates one example of a patient information screen 1200 that may be rendered 1102 in some examples. As shown in FIG. 12, the patient information screen 1200 includes a demographics control 1202, a medical history control 1204, an allergies control 1206, a medication control 1208, and other patient information control 1210.

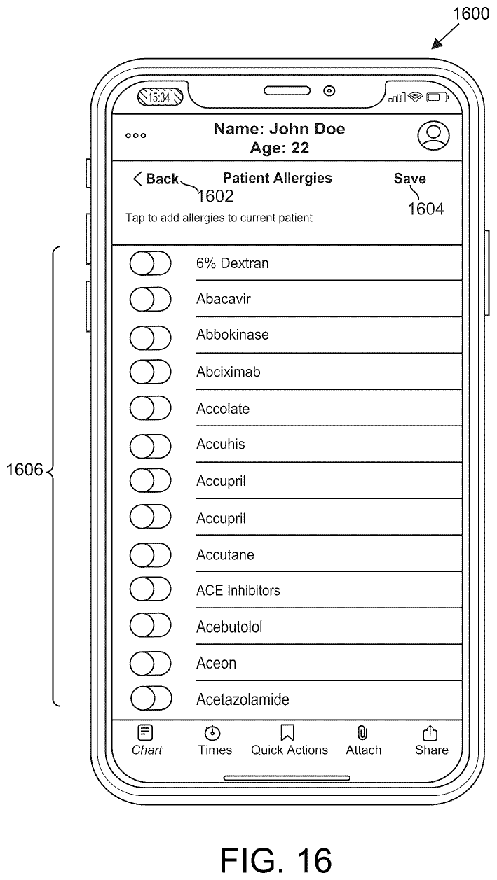

In some examples, the user can select the demographics control 1202 to navigate to a demographics screen useful to maintain ePCR data that documents demographic data regarding the patient. The user can select the medical history control 1204 to navigate to a medical history screen useful to maintain ePCR data that documents the patient's medical history. One example of a medical history screen 1400 is illustrated and further described with reference to FIG. 14. The user can select the allergies control 1206 to navigate to an allergies screen useful to maintain ePCR data that documents the patient's allergies. One example of an allergy screen 1600 is illustrated and further described with reference to FIG. 16. The user can select the medication control 1208 to navigate to a medication screen useful to maintain ePCR data that documents the medications prescribed to the patient. The user can select the other patient information control 1210 to navigate to an additional information screen useful to maintain ePCR data that documents other information regarding the patient, such as the patient's physician, immunization record, and information regarding the patient's relatives.

Returning to the process 300 with combined reference to FIGS. 11 and 12, the mobile EMS charting application receives 1104 input selecting a control. The mobile EMS charting application determines 1106 whether the demographics control 1202 was selected. If the demographics control 1202 was selected, the mobile EMS charting application interacts 1108 with the user to receive patient demographic data (e.g., via a demographics screen) and stores the received demographic data as ePCR data in the local data store. Subsequent to execution of the operation 1108, the mobile EMS charting application proceeds to the operation 1102. If the demographics control 1202 was not selected, the mobile EMS charting application proceeds to operation 1110.

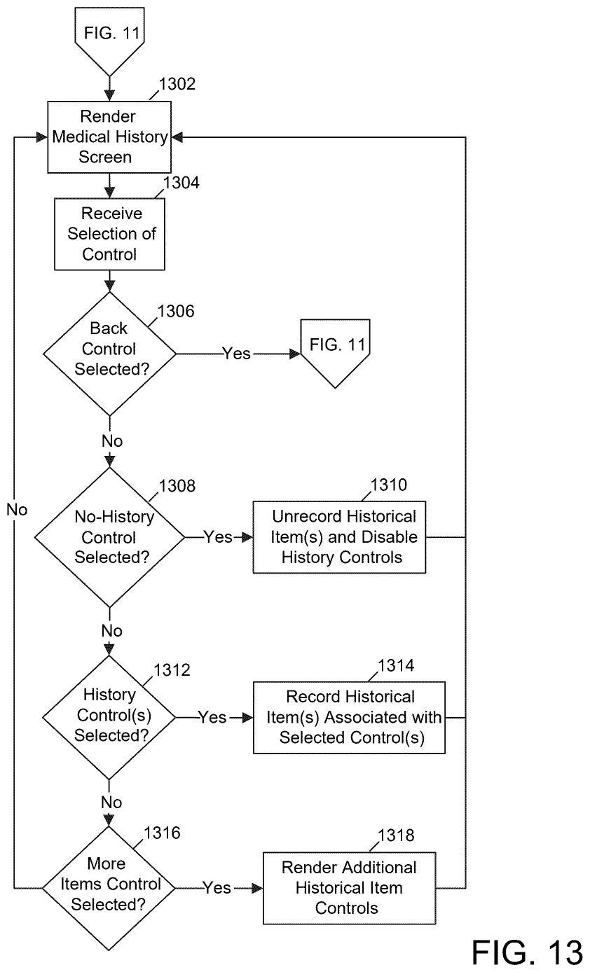

Continuing with the process 300, the mobile EMS charting application determines 1110 whether the medical history control 1204 was selected. If the medical history control 1204 was selected, the mobile EMS charting application proceeds to operation 1302 illustrated in FIG. 13. If the medical history control 1204 was not selected, the mobile EMS charting application proceeds to operation 1112.

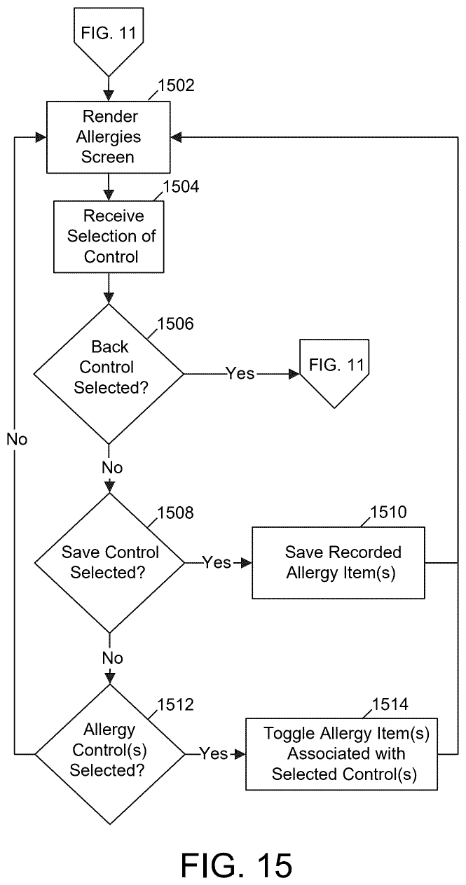

Continuing with the process 300, the mobile EMS charting application determines 1112 whether the allergies control 1206 was selected. If the allergies control 1206 was selected, the mobile EMS charting application proceeds to operation 1502 illustrated in FIG. 15. If the allergies control 1206 was not selected, the mobile EMS charting application proceeds to operation 1114.

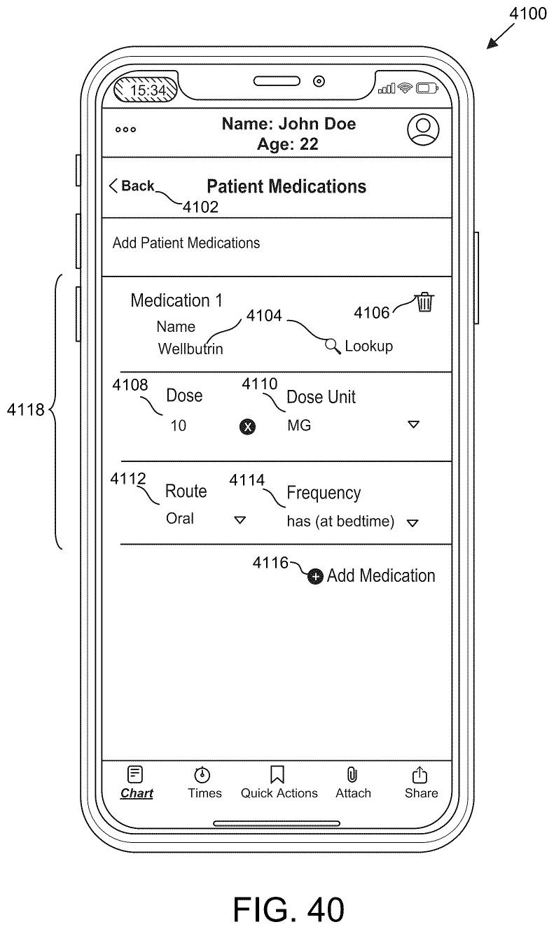

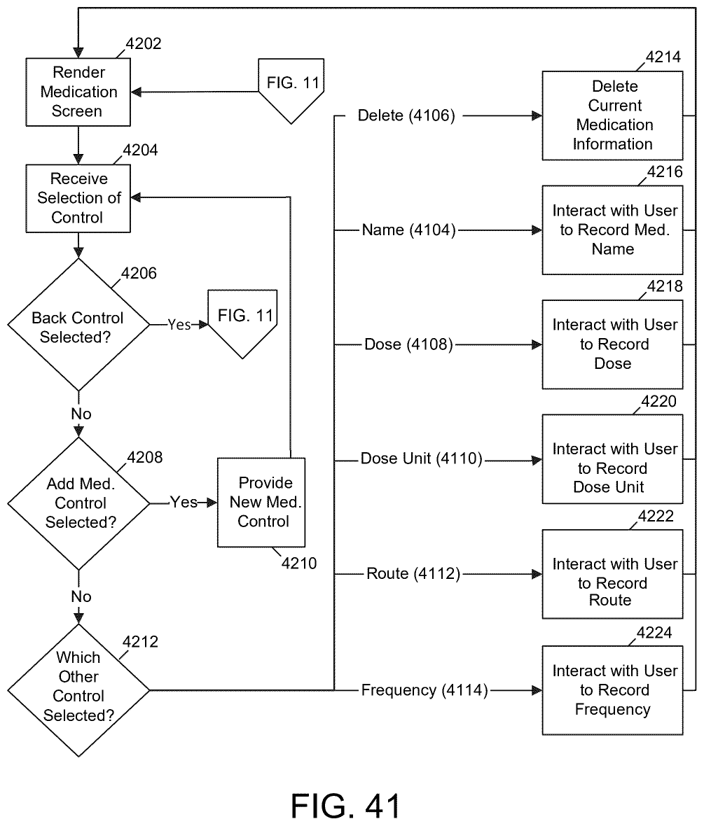

Continuing with the process 300, the mobile EMS charting application determines 1114 whether the medication control 1208 was selected. If the medication control 1208 was selected, the mobile EMS charting application proceed to operation 4202 illustrated in FIG. 41 and interacts 1116 with the user to receive patient medication data (e.g., via a medication screen illustrated in FIG. 40) and stores the received medication data as ePCR data in a local data store. Subsequent to execution of the operation 1116, the mobile EMS charting application proceeds to the operation 1102. If the medication control 1208 was not selected, the mobile EMS charting application proceeds to operation 1118.

Continuing with the process 300, the mobile EMS charting application determines 1118 whether the other patient information control 1210 was selected. If the other patient information control 1210 was selected, the mobile EMS charting application interacts 1120 with the user to receive the other patient data (e.g., via another patient information screen) and stores the received data as ePCR data in a local data store. Subsequent to execution of the operation 1120, the mobile EMS charting application proceeds to the operation 1102. If the other patient information control 1210 was not selected, the mobile EMS charting application proceeds to operation 1102.

Continuing with the process 300 with reference to FIG. 13, the mobile EMS charting application controls the host device to render 1302 a medical history screen within the user interface of the host device. FIG. 14 illustrates one example of a medical history screen 1400 that may be rendered 1302 in some examples. As shown in FIG. 14, the history screen 1400 includes a back control 1402, a no-history control 1404, a more history control 1406, and history controls 1408.

In some examples, the user can select the back control 1402 to navigate to the previously displayed screen. The user can select the no-history control 1404 to disable the more history control 1406 and the history controls 1408 and/or delete previously recorded ePCR data specifying the patient's medical history. The user can select one or more of the history controls 1408 to indicate that the patient's medical history includes the medical conditions associated with the selected history controls 1408. The user can select the more history control 1406 to navigate to another set of history controls 1408 associated with different medical conditions.

Returning to the process 300 with combined reference to FIGS. 13 and 14, the mobile EMS charting application receives 1304 input selecting a control. The mobile EMS charting application determines 1306 whether the back control 1402 was selected. If the back control 1402 was selected, the mobile EMS charting application proceeds to operation 1102 of FIG. 11. If the back control 1402 was not selected, the mobile EMS charting application proceeds to operation 1308.

Continuing with the process 300, the mobile EMS charting application determines 1308 whether the no-history control 1404 was selected. If the no-history control 1404 was selected, the mobile EMS charting application disables 1310 the more history control 1406 and the history controls 1408. Further, in some examples, the mobile EMS charting application deletes 1310 any previously recorded medical history for the patient from within the ePCR data stored in the local data store in response to selection of the no-history control 1404. If the no-history control 1404 was not selected, the mobile EMS charting application proceeds to operation 1312.

Continuing with the process 300, the mobile EMS charting application determines 1312 whether one of the history controls 1408 was selected. If one of the history controls 1408 was selected, the mobile EMS charting application toggles selection of the history control. If the toggle results in the history control being selected, the mobile EMS charting application associates 1314, within ePCR data housed in the local data store, the patient and the medical history item associated with the selected history control. If the toggle results in deselection of the history control, the mobile EMS charting application disassociates 1314, within ePCR data stored within the local data store, the patient and the medical history item associated with the deselected history control. If none of the history controls 1408 were selected, the mobile EMS charting application proceeds to operation 1312.

Continuing with the process 300, the mobile EMS charting application determines 1316 whether the more history control 1406 was selected. If the more history control 1406 was selected, the mobile EMS charting application controls the host device to render 1318 additional history controls for potential selection by the user. In some examples, the additional history controls can be comprehensive. If the more history control 1408 was not selected, the mobile EMS charting application proceeds to operation 1302.

It should be noted that, in some examples, the mobile EMS charting application is configured to position each of the history controls 1408 based on its frequency of use, with more frequently used controls being positioned toward the upper left of the history controls 1408. Alternatively or additionally, some examples of the mobile EMS charting application are configured to position the history controls 1408 based on other factors, such as statistical likelihood, specific regional configuration, EMS call type, chief complaint, etc. In an implementation, the mobile EMS charting application may be configured to allow customization of the history controls. For example, a medical director of an agency may configure and customize the history screens based on agency patient demographics, call types, etc.

Continuing with the process 300 with reference to FIG. 15, the mobile EMS charting application controls the host device to render 1502 an allergies screen within the user interface of the host device. FIG. 16 illustrates one example of an allergies screen 1600 that may be rendered 1502 in some examples. As shown in FIG. 16, the allergies screen 1600 includes a back control 1602, a save control 1604, and allergy controls 1606.

In some examples, the user can select the back control 1602 to navigate to the previously displayed screen. The user can select one or more of the allergy controls 1606 to indicate that the patient is allergic to the allergens associated with the selected allergy controls 1606. The user can select the save control 1604 to record ePCR data specifying that the patient's allergies include allergens associated with selected allergy controls.

Returning to the process 300 with combined reference to FIGS. 15 and 16, the mobile EMS charting application receives 1504 input selecting a control. The mobile EMS charting application determines 1506 whether the back control 1602 was selected. If the back control 1602 was selected, the mobile EMS charting application proceeds to operation 1102 of FIG. 11. If the back control 1602 was not selected, the mobile EMS charting application proceeds to operation 1508.

Continuing with the process 300, the mobile EMS charting application determines whether the save control 1604 was selected. If the save control 1604 was selected, the mobile EMS charting application stores 1510 associations, within ePCR data housed in the local data store, between the patient and all allergens associated with controls selected from the allergy controls 1606. If the save control 1604 was not selected, the mobile EMS charting application proceeds to operation 1512.

Continuing with the process 300, the mobile EMS charting application determines 1512 whether one of the allergy controls 1606 was selected. If one of the allergy controls 1606 was selected, the mobile EMS charting application toggles 1514 selection of the selected control. If none of the allergy controls 1606 were selected, the mobile EMS charting application proceeds to operation 1502.

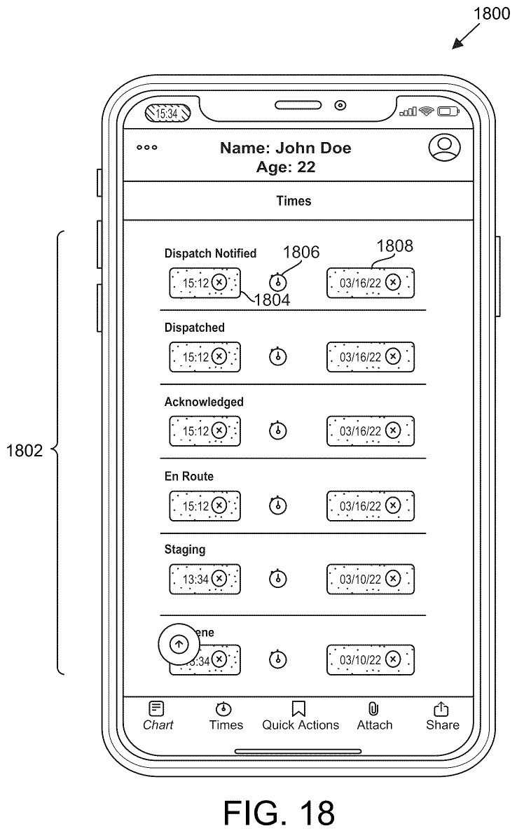

Continuing with the process 300 with reference to FIG. 17, the mobile EMS charting application controls the host device to render 1702 a times entry screen within the user interface of the host device. FIG. 18 illustrates one example of a times entry screen 1800 that may be rendered 1702 in some examples. As shown in FIG. 18, the times entry screen 1800 includes time and date controls 1802. The time and date controls include a current timestamp control 1806, dispatch notified time control 1804, and dispatch notified date control 1808.

In some examples, the user can interact with the time and date controls 1802 to specify dates and times of recordable events within the EMS call. For instance, in some examples, the user can interact with a time control and a date control (e.g., the dispatch notified time control 1804 and the dispatch notified date control 1808) to record ePCR data that specifies the time and date of an event in the past. Alternatively or additionally, the user can select a current timestamp control (e.g., the current timestamp control 1806) to record ePCR data that specifies an event occurred at the current time and date.

Returning to the process 300 with combined reference to FIGS. 17 and 18, the mobile EMS charting application receives 1704 input selecting a control. The mobile EMS charting application determines 1706 whether the dispatch notified time control 1804 was selected. If the dispatch notified time control 1804 was selected, the mobile EMS charting application interacts 1708 with the user to receive a time value and stores the time value in association with the dispatch notified event as ePCR data within the local data store. If the dispatch notified time control 1804 was not selected, the mobile EMS charting application proceeds to operation 1710.

Continuing with the process 300, the mobile EMS charting application determines 1710 whether the dispatch notified date control 1808 was selected. If the dispatch notified date control 1808 was selected, the mobile EMS charting application interacts 1712 with the user to receive a date value and stores the date value in association with the dispatch notified event as ePCR data within the local data store. If the dispatch notified date control 1808 was not selected, the mobile EMS charting application proceeds to operation 1714.

Continuing with the process 300, the mobile EMS charting application determines 1714 whether the current timestamp control 1806 was selected. If the current timestamp control 1806 was selected, the mobile EMS charting application stores 1716 the current time value and the current date value in association with the dispatch notified event as ePCR data within the local data store. If the current timestamp control 1806 was not selected, the mobile EMS charting application proceeds to operation 1702.

It should be appreciated that the operation illustrated in FIG. 17 can be applied to time controls, date controls, and current timestamp controls associated with other events, such as the events illustrated in FIG. 18.

Continuing with the process 300 with reference to FIG. 19, the mobile EMS charting application controls the host device to render 1902 a QAs screen within the user interface of the host device. FIG. 20 illustrates one example of a QAs screen 2000 that may be rendered 1902 in some examples. As shown in FIG. 20, the QAs screen 2000 includes a cardiac arrest QA control 2002A, a medical QA control 2002B, an RSI QA control 2002C, a trauma QA control 2002D, and a vascular access QA control 2002E. These particular controls are not limiting of the disclosure and other QA controls (e.g., a sepsis control, a respiratory distress control, etc.) may be configured in some implementations.

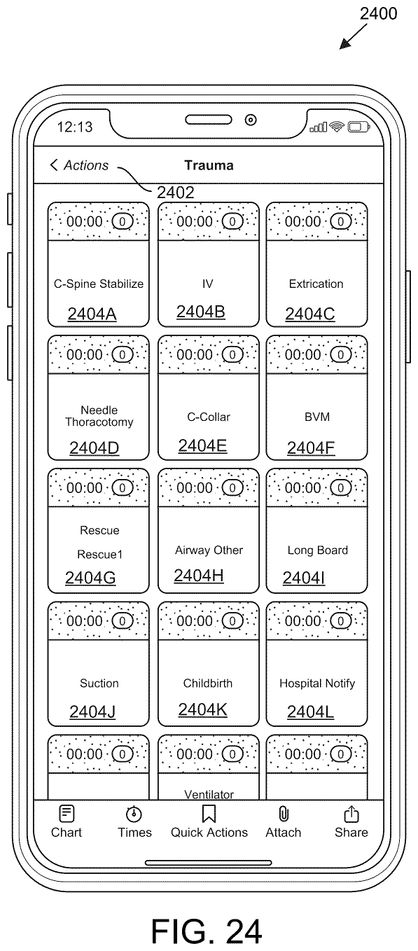

In some examples, the user can select the cardiac arrest QA control 2002A to navigate to a cardiac arrest QA screen useful to maintain ePCR data that documents actions performed by an EMS caregiver to treat a cardiac arrest suffered by the patient. One example of a cardiac arrest QA screen 2200 is illustrated and further described with reference to FIG. 22. The user can select the medical QA control 2002B to navigate to a medical QA screen useful to maintain ePCR data that documents actions performed by an EMS caregiver to treat the patient medically. The user can select the RSI QA control 2002C to navigate to an RSI QA screen useful to maintain ePCR data that documents actions performed by an EMS caregiver during rapid sequence intubation of the patient. The user can select the trauma QA control 2002D to navigate to a trauma QA screen useful to maintain ePCR data that documents actions performed by an EMS caregiver to treat trauma in the patient. One example of a trauma QA screen 2400 is illustrated and further described with reference to FIG. 24. The user can select the vascular access QA control 2002E to navigate to a vascular access QA screen useful to maintain ePCR data that documents actions performed by an EMS caregiver during vascular access of the patient.

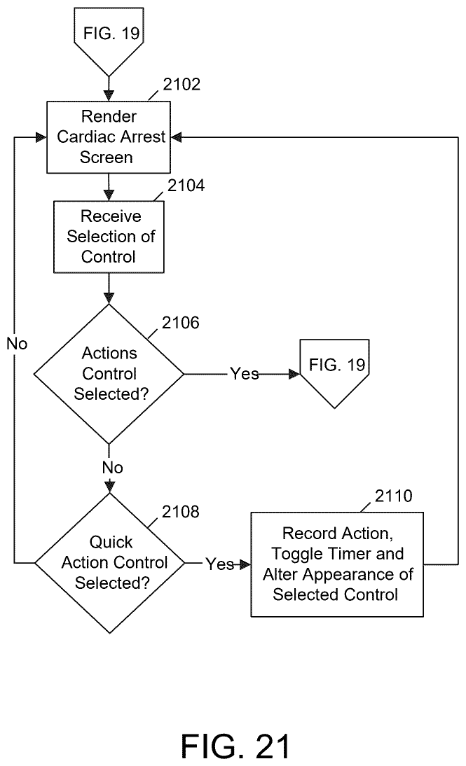

Returning to the process 300 with combined reference to FIGS. 19 and 20, the mobile EMS charting application receives 1904 input selecting a control. The mobile EMS charting application determines 1906 whether the cardiac arrest QA control 2002A was selected. If the cardiac arrest QA control 2002A was selected, the mobile EMS charting application proceeds to operation 2102 illustrated in FIG. 21. If the cardiac arrest QA control 2002A was not selected, the mobile EMS charting application proceeds to operation 1908.

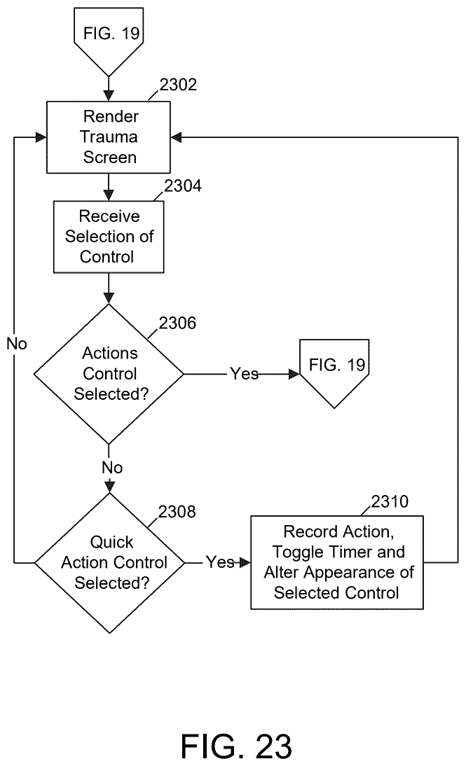

Continuing with the process 300, the mobile EMS charting application determines 1908 whether the trauma QA control 2002D was selected. If the trauma QA control 2002D was selected, the mobile EMS charting application proceeds to operation 2302 illustrated in FIG. 23. If the trauma QA control 2002D was not selected, the mobile EMS charting application proceeds to operation 1910.

Continuing with the process 300, the mobile EMS charting application determines 1910 whether another QA control (e.g., any of controls 2002B, 2002C, or 2002E) was selected. If another QA control was selected, the mobile EMS charting application initiates 1912 a screen associated with the selected control. The screen includes QA controls relevant to procedures and interventions associated with the selected QA control.

It should be noted that, in some examples, the mobile EMS charting application can be configured to create QA controls and screens other than those described herein. For instance, in some examples, QA controls and screens are created for EMS call types encountered within a particular geographic region or EMS agency.

Continuing with the process 300 with reference to FIG. 21, the mobile EMS charting application controls the host device to render 2102 a cardiac arrest QA screen within the user interface of the host device. FIG. 22 illustrates one example of a cardiac arrest QA screen 2200 that may be rendered 2102 in some examples. As shown in FIG. 22, the cardiac arrest QA screen 2200 includes an actions control 2202 and QA controls 2204A-2204L.

In some examples, the user can select the actions control 2202 to navigate to the previous screen (e.g., the QAs screen 2000 of FIG. 20). The user can select one or more of the QA controls 2204A-2204L to document an action performed by the EMS caregiver. In response to receiving input selecting one of the QA controls 2204A-2204L, the mobile EMS charting application may highlight the control and iteratively display time elapsed since the control was last selected.

Returning to the process 300 with combined reference to FIGS. 21 and 22, the mobile EMS charting application receives 2104 input selecting a control. The mobile EMS charting application determines 2106 whether the actions control 2202 was selected. If the actions control 2202 was selected, the mobile EMS charting application proceeds to operation 1902 illustrated in FIG. 19. If the actions control 2202 was not selected, the mobile EMS charting application proceeds to operation 2108.

Continuing with the process 300, the mobile EMS charting application determines 2108 whether one of the QA controls 2204A-2204L was selected. If one of the QA controls 2204A-2204L was selected, the mobile EMS charting application executes 2110 a sequence of operations. This sequence includes recording the action as ePCR data within the local data store, toggling a configurable timer associated with the selected control, and altering the appearance of the selected control. If none of the QA controls 2204A-2204L are selected, the mobile EMS charting application proceeds to operation 2102.

The graphical elements 2206A, 2206D, 2208J, 2208A, and 2208D illustrate alterations the mobile EMS charting application is configured to effect in some examples. As shown in FIG. 22, the blue header 2206A indicates that the QA control 2204A is toggled off and the integer 1 within the white circle of the header indicates that the QA control 2204A has been toggled on once. The timestamp 2210A indicates the time when the QA control 2204A was toggled on. The time duration indicated in the blue header 2206A indicates that the QA control was toggled off 2 minutes and 10 seconds after it was toggled on. The gray header 2206D indicates that the QA control 2204D is toggled on and has a timer active that has not transgressed a configurable threshold duration specific to the QA control 2204D. The red header 2206J indicates that the QA control 2204J is toggled on and that its timer has transgressed a configurable threshold duration (e.g., 2 minutes).

The timestamps 2210A presented by a QA control can aid a user in tracking timing details for repeated or follow-up activities specified within emergency response and treatment protocols, so that the user does not have to track times individually. Thus the QA control helps the user to perform prescribed protocols accurately. The highlighting presented by the QA controls, e.g. the red header 2206J, supports the user by enabling quick recognition of actions that may need tending to. With these QA controls as well as other GUI features, (e.g., single touch timers, medical history controls, medication scanning, customization, etc.), the mobile EMS charting application described herein supports and improves the care provided by EMS. Thus the mobile EMS charting application not only provides efficient and intuitive recordation tools for an ePCR but also provides the user with added value by assisting with protocol adherence and workflow management.

It should be noted that, in some examples, the mobile EMS charting application is further configured to notify the user of expired times using other mechanisms, such as in-app notifications, push notifications, and the like. In an implementation, the mobile EMS charting application 220 may leverage cellular network communications to enable notifications, reminders, and other smartphone capabilities like texting, location services (e.g., via a GPS and/or cellular location determination) and may interact with one or more native smartphone applications.

Continuing with the process 300 with reference to FIG. 23, the mobile EMS charting application controls the host device to render 2302 a trauma QA screen within the user interface of the host device. FIG. 24 illustrates one example of a trauma QA screen 2400 that may be rendered 2302 in some examples. As shown in FIG. 24, the trauma QA screen 2400 includes an actions control 2402 and QA controls 2404A-2404L.

In some examples, the user can select the actions control 2402 to navigate to the previous screen (e.g., the QAs screen 2000 of FIG. 20). The user can select one or more of the QA controls 2404A-2404L to document an action performed by the EMS caregiver. In response to receiving input selecting one of the QA controls 2404A-2404L, the mobile EMS charting application may highlight the control and iteratively display time elapsed since the control was last selected.

Returning to the process 300 with combined reference to FIGS. 23 and 24, the mobile EMS charting application receives 2304 input selecting a control. The mobile EMS charting application determines 2306 whether the actions control 2402 was selected. If the actions control 2402 was selected, the mobile EMS charting application proceeds to operation 1902 illustrated in FIG. 19. If the actions control 2402 was not selected, the mobile EMS charting application proceeds to operation 2308.

Continuing with the process 300, the mobile EMS charting application determines 2308 whether one of the QA controls 2404A-2404L was selected. If one of the QA controls 2404A-2404L was selected, the mobile EMS charting application executes 2310 a sequence of operations. This sequence includes recording the action as ePCR data within the local data store, toggling a configurable timer associated with the selected control, and altering the appearance of the selected control. If none of the QA controls 2404A-2404L are selected, the mobile EMS charting application proceeds to operation 2302.

Continuing with the process 300 with reference to FIG. 25, the mobile EMS charting application controls the host device to render 2502 an attachments screen within the user interface of the host device. FIG. 26 illustrates one example of an attachments screen 2600 that may be rendered 2502 in some examples. As shown in FIG. 26, the attachments screen 2600 includes a patient identification document scan control 2602 (e.g., for use in scanning a driver's license or other patient identification documentation), a signature control 2604, a file control 2606, an upload control 2608, and a display attachments control 2610.

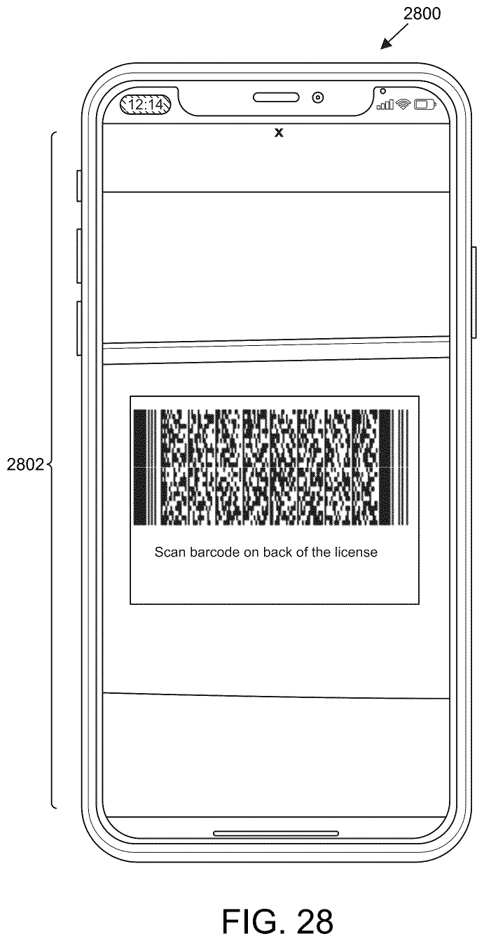

In some examples, the user can select the scan control 2602 to navigate to a scan screen useful to acquire patient demographic data from the patient's driver's license or other patient identification documentation. One example of a scan screen 2800 is illustrated and further described with reference to FIG. 28. The user can select the add signatures control 2604 to navigate to a signatures screen useful to acquire signatures of individuals involved in the EMS call. One example of a signatures screen 3100 is illustrated and further described with reference to FIG. 31. The user can select the choose file control 2606 to navigate to a screen that enables the user to select one or more files to attach to the ePCR and upload to the remote server. The user can select the upload control 2608 to initiate upload of currently selected files. The user can select the display attachments control 2610 to toggle viewing of any attachments already locally stored.

Returning to the process 300 with combined reference to FIGS. 25 and 26, the mobile EMS charting application receives 2504 input selecting a control. The mobile EMS charting application determines 2506 whether the scan control 2602 was selected. If the scan control 2602 was selected, the mobile EMS charting application proceeds to operation 2702 illustrated in FIG. 27. If the scan control 2602 was not selected, the mobile EMS charting application proceeds to operation 2508.

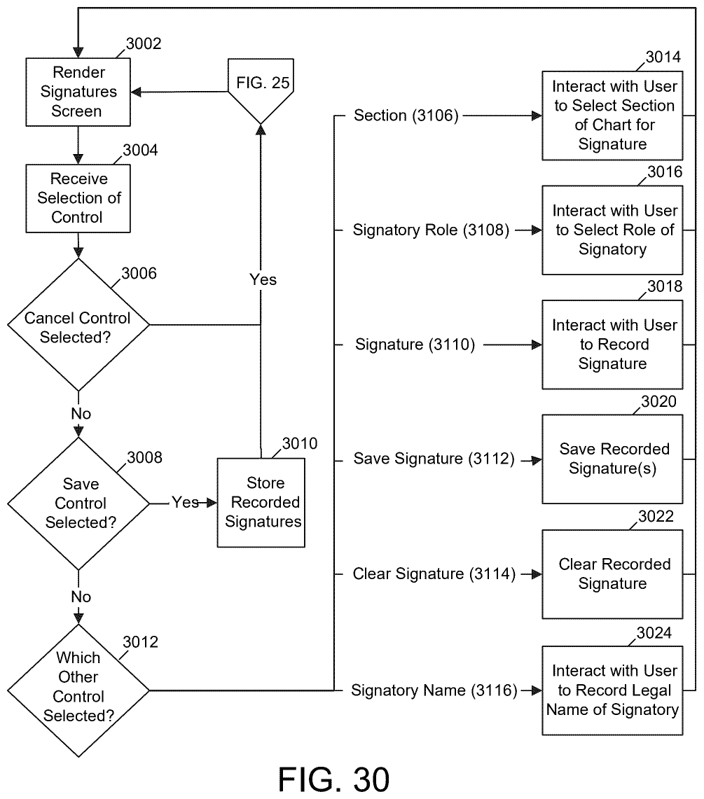

Continuing with the process 300, the mobile EMS charting application determines 2508 whether the signature control 2604 was selected. If the signature control 2604 was selected, the mobile EMS charting application proceeds to operation 3002 illustrated in FIG. 30. If the signature control 2604 was not selected, the mobile EMS charting application proceeds to operation 2510.

Continuing with the process 300, the mobile EMS charting application determines 2510 whether the file control 2606 was selected. If the file control 2606 was selected, the mobile EMS charting application interacts 2512 (e.g., via a file selection screen) with the user to receive an identifier of a file that is accessible via the host device. The mobile EMS charting application stores file identifiers received via the file control 2606 as identifiers of attachments to the ePCR in the local data store. If the file control 2606 was not selected, the mobile EMS charting application proceeds to operation 2514.

Continuing with the process 300, the mobile EMS charting application determines 2514 whether the upload control 2608 was selected. If the upload control 2608 was selected, the mobile EMS charting application uploads 2516 to the remote server, as attachments to the ePCR, one or more files previously identified via the file control 2606. If the upload control 2608 was not selected, the mobile EMS charting application proceeds to operation 2518.

Continuing with the process 300, the mobile EMS charting application determines 2518 whether the hide control 2610 was selected. If the hide control 2610 was selected, the mobile EMS charting application toggles 2520 the hide control 2610 between hiding identifiers of attachments and displaying identifiers of attachments. If the hide control 2610 was not selected, the mobile EMS charting application proceeds to operation 2502.

Continuing with the process 300 with reference to FIG. 27, the mobile EMS charting application controls the host device to render 2702 a scan screen within the user interface of the host device. FIG. 28 illustrates one example of a scan screen 2800 that may be rendered 2702 in some examples. As shown in FIG. 28, the scan screen 2800 includes a viewfinder control 2802.

In some examples, the user can manipulate the host device or the driver's license or other patient identification documentation to position a fiducial of the driver's license or other patient identification documentation within the active area of the viewfinder control 2802 to initiate the scanning thereof.

Continuing with the process 300 with combined reference to FIGS. 27 and 28, the mobile EMS charting application controls a camera of the host device to acquire images and attempts to identify 2704 a fiducial within the acquired images. If a fiducial is identified, the mobile EMS charting application proceeds to operation 2705. If a fiducial is not identified, the mobile EMS charting application proceeds to operation 2702.

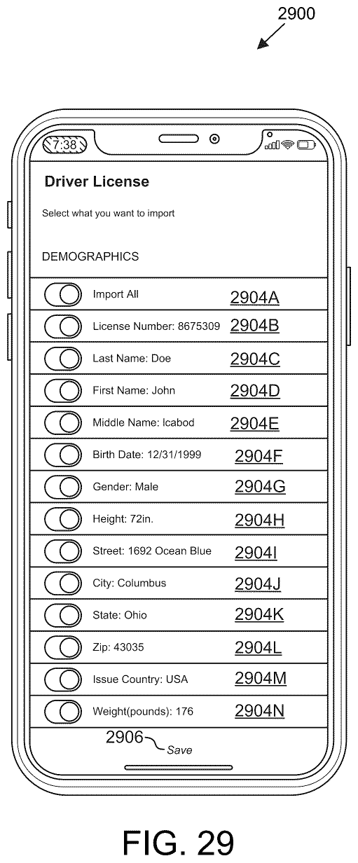

Continuing with the process 300, the mobile EMS charting application extracts 2705 demographic data from the fiducial by, for example, decoding the fiducial, parsing the decoded fiducial to identify demographic data fields specified therein, and storing the demographic data fields in local memory. In some examples, the operation 2705 may further include API-based interoperations involving the mobile EMS charting application and one or more other elements of a SaaS platform (e.g., the SaaS platform described below with reference to FIG. 37A). For instance, in some implementations, the mobile EMS charting application may provide the extracted demographic data (e.g., first name, last name, address, date of birth, and/or other demographic data of the patient) to a data analytics platform 951 (e.g., as provided by the data analytics system server(s) 952 as shown in FIG. 37A and as described in regard to FIG. 37B). The data analytics platform 951 may apply predictive analytics to data accessed from a plurality of data sources 3770 to discover more and/or corrected or alternative demographic data (e.g., social security number, aliases or other former names) prior to proceeding to the operation 2706. In some implementations, the data analytics platform may perform other analytic functions including, for example, but not limited to, insurance discovery (e.g., finding insurance coverage for a patient that the patient or biller may not have realized), insurance verification, predictive estimates of a patient's ability to pay which may include deductible or prior authorization information, to name a few examples. Insurance discovery by the data analytics platform may enable the data analytics platform to provide insurance identification information for insurance coverage found by the data analytics system. Next, the mobile EMS charting application controls the host device to render 2706 a demographics screen within the user interface of the host device. FIG. 29 illustrates one example of an importation screen 2900 for the driver's license or other patient identification documentation that may be rendered 2706 in some examples. As shown in FIG. 29, the importation screen 2900 includes demographic item controls 2904A-2904N and a save control 2906.

In some examples, the user can select of any one of the item controls 2904A-2904N to toggle between selection and deselection of the control. The user can select the save control 2906 to store acquired values associated with selected demographic item controls as ePCR data in the local data store.

Continuing with the process 300 with combined reference to FIGS. 27 and 29, the mobile EMS charting application receives 2708 input selecting a control. The mobile EMS charting application determines 2710 whether one of the item controls 2904A-2904N was selected. If one of the item controls 2904A-2904N was selected, the mobile EMS charting application toggles 2712 the control between selection and deselection. If none of the item controls 2904A-2904N was selected, the mobile EMS charting application proceeds to operation 2714.

Continuing with the process 300, the mobile EMS charting application determines 2714 whether the save control 2906 was selected. If the save control 2906 was selected, the EMS charting stores 2716 the demographic data field values associated with the currently selected item controls 2904A-2904N as ePCR data in the local data store. Subsequent to the operation 2716, the mobile EMS charting application proceeds to the operation 2502 of FIG. 25. If the save control 2906 was not selected, the mobile EMS charting application proceeds to the operation 2706.

In some examples, the mobile EMS charting application is configured to toggle selection of all of the item controls 2904A-2904N in response to selection of the item control 2904A.

Continuing with the process 300 with reference to FIG. 30, the mobile EMS charting application controls the host device to render 3002 a signatures screen within the user interface of the host device. FIG. 31 illustrates one example of a signatures screen 3100 that may be rendered 3002 in some examples. As shown in FIG. 31, the signatures screen 3100 includes a cancel control 3102, a save control 3104, a responsible section control 3106, a role control 3108, a signature control 3110, a save signature control 3112, a clear control 3114, and a printed name control 3116.

In some examples, the user can select the cancel control 3102 to abort signature capture and navigate to the previous screen (e.g., the attachments screen 2600 of FIG. 26). The user can select the save control 3104 to complete signature capture and store capture signature as ePCR data in a local data store. The user can interact with the responsible section control 3106 to select a section of a document for which the signatory is responsible. The user can interact with the role control 3108 to select a capacity in which the signatory is acting. The user can interact with the signature control 3110 to record a signature (e.g., via a stylus or finger/touch drawing). The user can select the save signature to store an image of the signature as ePCR data in a local data store. The user can select the clear control to erase a previous, unsatisfactory signature. The user can interact with the printed name control 3116 to input the name of the signatory.

Returning to the process 300 with combined reference to FIGS. 30 and 31, the mobile EMS charting application receives 3004 input selecting a control. The mobile EMS charting application determines 3006 whether the cancel control 3102 was selected. If the cancel control 3102 was selected, the mobile EMS charting application proceeds to the operation 2502 of FIG. 25. If the cancel control 3102 was not selected, the mobile EMS charting application proceeds to operation 3008.

Continuing with the process 300, the mobile EMS charting application determines 3008 whether the save control 3104 was selected. If the save control 3104 was selected, the mobile EMS charting application stores 3010 signature information previously acquired via the current instance of the screen 3100 as ePCR data in the local data store. If the save control 3104 was not selected, the mobile EMS charting application proceeds to operation 3012.

Continuing with the process 300, the mobile EMS charting application determines 3012 which other control was selected. If the section control 3106 was selected, the mobile EMS charting application interacts 3014 with the user to receive an identifier of the ePCR section to which a signature pertains. If the signatory role control 3108 was selected, the mobile EMS charting application interacts 3016 with the user to receive an identifier of the official capacity under which the signature is given. If the signature control 3110 was selected, the mobile EMS charting application interacts 3018 with the user to receive and record the user's signature (e.g., via a stylus, finger, etc.). If the save signature control 3112 was selected, the mobile EMS charting application saves 3020 the recorded signature as ePCR data in the local data store. If the clear signature control 3114 was selected, the mobile EMS charting application clears 3022 any signature presently displayed within the signature control 3110. If the printed name control 3116 was selected, the mobile EMS charting application interacts 3024 with the receive the signatory's name in text.

Continuing with the process 300 with reference to FIG. 32, the mobile EMS charting application controls the host device to render 3202 a share screen within the user interface of the host device. FIG. 33 illustrates one example of a share screen 3300 that may be rendered 3202 in some examples. As shown in FIG. 32, the share screen 3300 control includes a submit control 3302 and a share control 3304.

In some examples, the user can select the submit control 3302 to upload the ePCR to the remote server. Upon a successful upload, the mobile EMS charting application displays information identifying the ePCR in the share control 3304. As shown in FIG. 33, this information can include a serial number, a URL, and/or a fiducial through which other instances of the mobile EMS charting application may access the ePCR via the remote server.

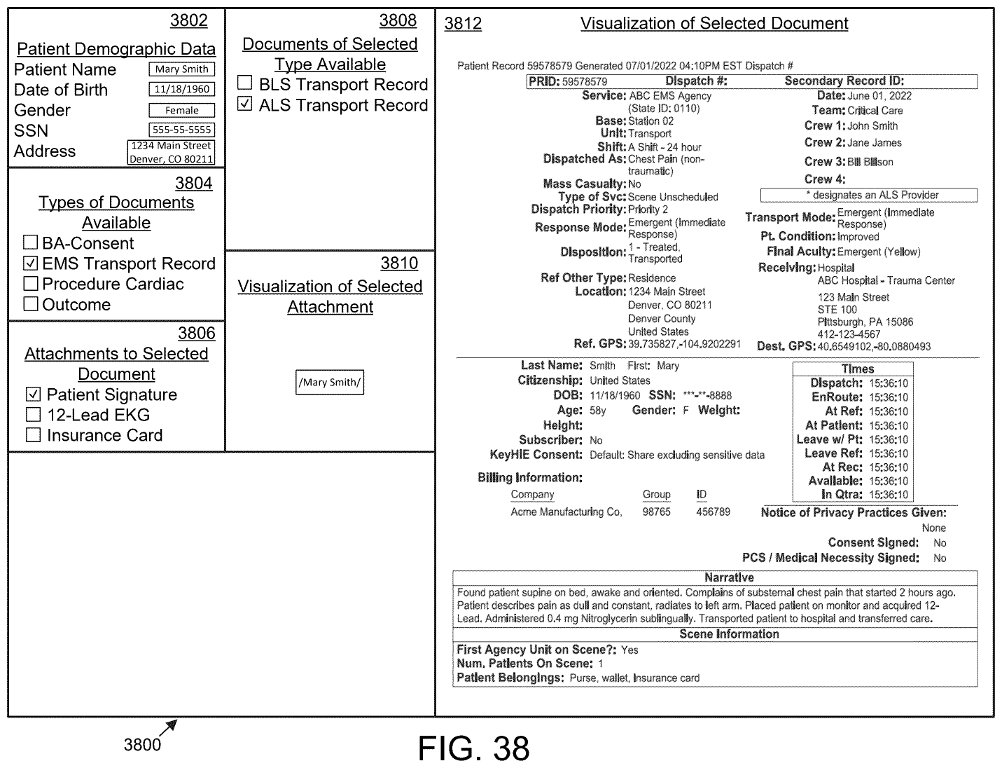

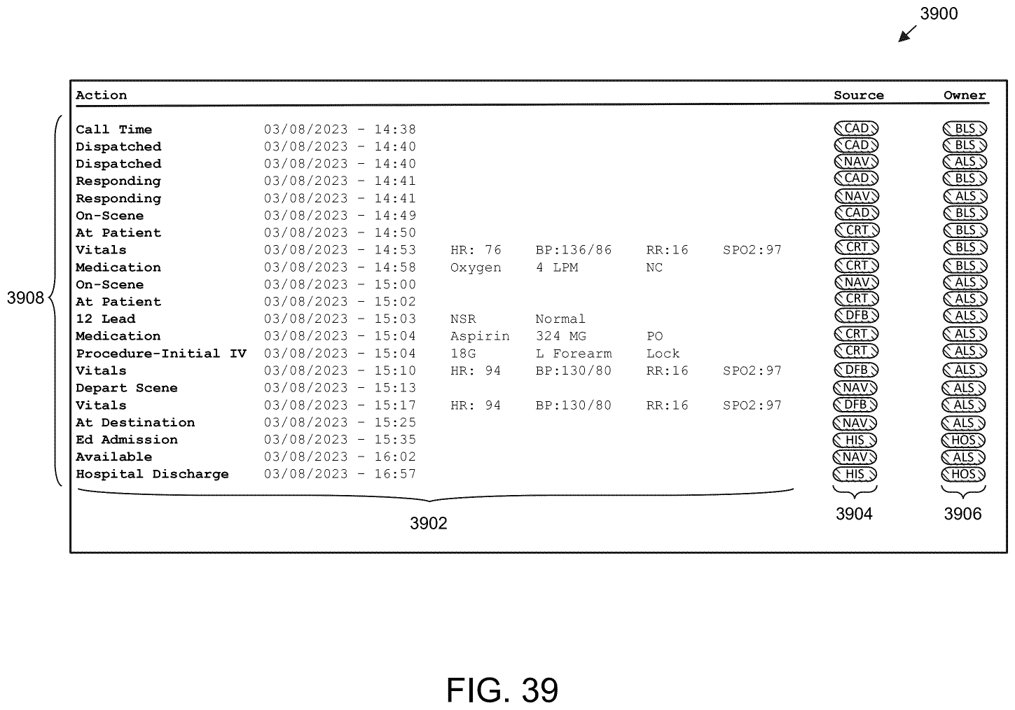

Returning to the process 300 with combined reference to FIGS. 32 and 33, the mobile EMS charting application receives 3204 input selecting a control. The mobile EMS charting application determines 3206 whether the submit control 3302 was selected. If the submit control 3302 was selected, the mobile EMS charting application validates 3208 the ePCR data for the open ePCR, uploads the ePCR data to the remote server, and controls the host device to render identification information of the uploaded ePCR within the share control 3304. This identification information can include, for example, a serial number, a URL, and/or a fiducial through which other instances of the mobile EMS charting application may access the ePCR data via the remote server. For instance, in some examples, the uploaded ePCR data is accessible by hospital personnel and other users via the remote server through the screens illustrated in FIGS. 38 and 39, which are described further below. Additionally or alternatively, in some examples, the ePCR data is uploaded in a standard format (e.g., HL7) to the hospital's information system (system(s) 3772 and/or repository 3705) using features of the SaaS platform and the integration platform as described herein with reference to FIGS. 37 and 40. If the submit control 3302 was not selected, the mobile EMS charting application proceeds to the operation 3202.

It should be noted that the validation executed by the mobile EMS charting application can include checking for population of required data fields and/or ensuring that the populated value is acceptable.

Continuing with the process 300 with reference to FIG. 41, the mobile EMS charting application controls the host device to render 4202 a medication screen within the user interface of the host device. FIG. 40 illustrates one example of a medications screen 4100 that may be rendered 4202 in some examples. As shown in FIG. 40, the medications screen 4100 includes a back control 4102, a delete control 4106, an add medication control 4116 and a medication control group 4118 that includes a name control 4104, a dose control 4108, a dose unit control 4110, a route control 4112, and a frequency control 4114.

In some examples, the user can select the back control 4102 to navigate to the previous screen. The user can interact with the name control 4104 to specify and store a medication of the patient as ePCR data in a local data store. The user can select the delete control 4106 to remove the medication information of the patient that is currently selected and displayed in the medication control group 4118 from the ePCR data in the local data store. The medication control group 4118 is associated with and specifies attributes of a medication taken by or otherwise associated with a patient. The user can interact with the dose control 4108 to specify a dose amount for the medication currently selected and displayed in the medication control group 4118. The user can interact with the dose unit control 4110 to record units in which the dose is specified. The user can interact with the route control 4112 to specify the route by which the medication is taken. The user can interact with the frequency control 4114 to specify the frequency with which the medication is taken. The user can select the add medication control 4116 to access another medication control group to specify another medication taken by the patient.

Returning to the process 300 with combined reference to FIGS. 40 and 41, the mobile EMS charting application receives 4204 input selecting a control. The mobile EMS charting application determines 4206 whether the back control 4102 was selected. If the back control 4102 was selected, the mobile EMS charting application proceeds to the operation 1102 of FIG. 11. In some examples, the mobile EMS charting application stores, as ePCR data in association with the patient in a local data store, all medication information currently entered in the screen 4100 prior to proceeding to the operation 1102 of FIG. 11. If the back control 4102 was not selected, the mobile EMS charting application proceeds to operation 4208.

Continuing with the process 300, the mobile EMS charting application determines 4208 whether the add medication control 4116 was selected. If the add medication control 4116 was selected, the mobile EMS charting application provides 4210 another set of medication controls (e.g., controls 4104-4114 of the medication control group 4118) to enable the user to enter information regarding the additional medication. If the add medication control 4116 was not selected, the mobile EMS charting application proceeds to operation 4212.

Continuing with the process 300, the mobile EMS charting application determines 4212 which other control was selected. If the delete control 4106 was selected, the mobile EMS charting application removes 4214, from the local data store, medication information regarding the medication specified by the medication control group 4118. If the name control 4104 was selected, the mobile EMS charting application interacts 4216 with the user to identify and record a medication name in association with the patient as ePCR data within a local data store. This interaction may include receiving user input (e.g., gestures) spelling the medication name and/or receiving search terms (e.g., partial names), depending on whether the user selects the text box or the search tool icons within the name control 4104. If the dose control 4108 was selected, the mobile EMS charting application interacts 4218 with the user to record, as ePCR data in a local data store, a dose amount for the medication specified by the medication control group 4118. If the dose unit control 4110 was selected, the mobile EMS charting application interacts 4220 with the user to record, as ePCR data in a local data store, a dose unit for the currently specified dose amount. If the route control 4112 was selected, the mobile EMS charting application interacts 4222 with the user to record, as ePCR data in a local data store, a route of administration of the medication specified by the medication control group 4118. If the frequency control 4114 was selected, the mobile EMS charting application interacts 4224 with the user to record, as ePCR data in a local data store, a frequency with which the medication specified by the medication control group 4118 is administered.

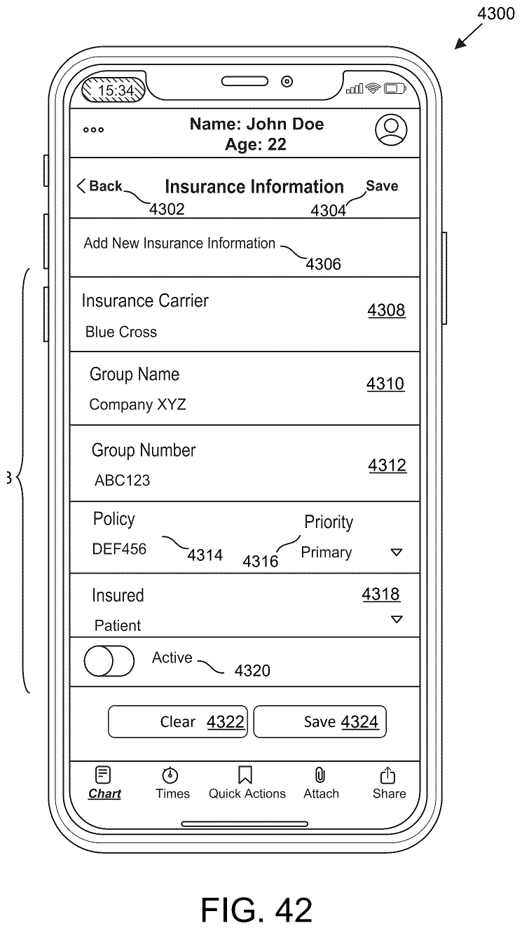

Continuing with the process 300 with reference to FIG. 43, the mobile EMS charting application controls the host device to render 4402 an insurance screen within the user interface of the host device. FIG. 42 illustrates one example of an insurance screen 4300 that may be rendered 4402 in some examples. As shown in FIG. 42, the insurance screen 4300 includes a back control 4302, save controls 4304 and 4324, a clear control 4322, an add insurance control 4306 and an insurance control group 4328 that includes a carrier control 4308, a group name control 4310, a group number control 4312, a policy control 4314, a priority control 4316, an insured control 4318, and an active control 4320. The insurance control group 4328 is associated with and specifies attributes of an insurance policy held by or otherwise associated with a patient.

In some examples, the user can select the back control 4302 to navigate to the previous screen. The user can select the save control 4304 to store, as ePCR data in a local data store, the insurance information of the patient that is currently entered and displayed in the insurance control group 4328. The user can select the add insurance control 4306 to access another insurance control group to specify additional insurance information for the patient. The user can interact with the carrier control 4308 to specify an insurance carrier for the patient. The user can interact with the group name control 4310 to specify a group name for patient's insurance policy. The user can interact with the group number control 4312 to specify a group number for the patient's insurance policy. The user can interact with the policy control 4314 to specify a policy identifier for the patient's insurance policy. The user can interact with the priority control 4316 to specify the priority (primary, secondary, etc.) of the insurance policy of the patient specified in the insurance control group 4328. The user can interact with the insured control 4318 to specify the holder of the patient's insurance policy. The user can select the active control 4320 to toggle the status of the currently selected insurance policy specified in the insurance control group 4328 between active and inactive. The user can select the clear control 4322 to remove the patient insurance information currently selected and displayed in the insurance control group 4328 from the ePCR data in the local data store.

Returning to the process 300 with combined reference to FIGS. 42 and 43, the mobile EMS charting application receives 4404 input selecting a control. The mobile EMS charting application determines 4406 whether the back control 4302 was selected. If the back control 4302 was selected, the mobile EMS charting application proceeds to the operation 702 of FIG. 7. If the back control 4302 was not selected, the mobile EMS charting application proceeds to operation 4408.

Continuing with the process 300, the mobile EMS charting application determines 4408 whether the add insurance control 4306 was selected. If the add insurance control 4306 was selected, the mobile EMS charting application provides 4410 another set of insurance controls (e.g., controls 4308-4320 of the insurance control group) to enable the user to enter information regarding the additional insurance policy. If the add insurance control 4306 was not selected, the mobile EMS charting application proceeds to operation 4412.

Continuing with the process 300, the mobile EMS charting application determines 4412 whether the save control 4304 was selected. If the save control 4304 was selected, the mobile EMS charting application stores 4414 associations, within ePCR data housed in the local data store, the insurance information entered in the screen 4300 and associations between the patient and insurance information. If the save control 1604 was not selected, the mobile EMS charting application proceeds to operation 4416.

Continuing with the process 300, the mobile EMS charting application determines 4416 which other control was selected. If the carrier control 4308 was selected, the mobile EMS charting application interacts 4418 with the user to identify and record, as ePCR data within a local data store, a carrier name in association with the insurance policy specified by the insurance control group 4328. If the group name control 4310 was selected, the mobile EMS charting application interacts 4420 with the user to identify and record, as ePCR data within a local data store, a group name in association with the insurance policy specified by the insurance control group 4328. If the group number control 4312 was selected, the mobile EMS charting application interacts 4422 with the user to identify and record, as ePCR data within a local data store, a group number in association with the insurance policy specified by the insurance control group 4328. If the policy control 4314 was selected, the mobile EMS charting application interacts 4424 with the user to identify and record, as ePCR data within a local data store, a policy identifier in association with the insurance policy specified by the insurance control group 4328. If the priority control 4316 was selected, the mobile EMS charting application interacts 4426 with the user to identify and record, as ePCR data within a local data store, a priority (e.g., primary, secondary, etc.) identifier in association with the insurance policy specified by the insurance control group 4328. This interaction may include, for example, selection from a list of predefined priorities. If the insured control 4318 was selected, the mobile EMS charting application interacts 4428 with the user to identify and record, as ePCR data within a local data store, an identifier of the holder of a patient's insurance policy in association with the insurance policy specified by the insurance control group 4328. This interaction may include, for example, selection from a list of predefined holder identifiers (patient, parent, guardian, etc.). If the active control 4320 was selected, the mobile EMS charting application interacts 4430 with the user to toggle and record, as ePCR data within a local data store, an indicator of whether the insurance policy specified by the insurance control group 4328 is active and inactive. If the clear control 4322 was selected, the mobile EMS charting application removes 4432, from the local data store, information regarding the insurance policy specified by the insurance control group 4328.

In an implementation, the mobile EMS charting application may interface with the data analytics platform 951 (e.g., as described in regard to FIG. 37B) to automatically populate, verify, and/or supplement insurance information. For example, the charting application may provide demographic, insurance or other patient information to the data analytics platform 951. The data analytics platform 951 may then use predictive intelligence tools including, for example, insurance verification and/or insurance discovery, based on the data available in the data sources 3770 to verify or supplement the insurance information and provide the verified and/or supplemented information back to the charting application for population of the charting application fields. For example, the insurance verification information may include an indication that one or more insurance policies are currently active and in effect and/or information about specific coverage (e.g., procedures, reimbursement limits, provider requirements, etc.).

In some examples, the mobile EMS charting application may interoperate with the data analytics platform 951 to obtain insurance information specifying one or more of the following characteristics of an insurance policy held by the patient: carrier name, group name, group number, policy identifier, policy holder, or policy status (e.g., active or inactive). Further, in some examples, the mobile EMS charting application may interoperate with the the data analytics platform 951 to obtain insurance information specifying one or more additional insurance policies applicable to the patient encounter and the policy priority (e.g., primary, secondary, tertiary, etc.) of each.

FIGS. 1A-1F, 4, 6A, 6B, 8, 10, 12, 14, 16, 18, 20, 22, 24, 26, 28, 29, 31, 33, 40, and 42 collectively illustrate a GUI presented by a mobile EMS charting application in some examples. Other examples are possible without departing from the scope of this disclosure. For instance, in one example, the screens illustrated in the FIGS. listed above are capable of being rendered within a dark mode that emits less light and, thereby, causes little or no adjustment to the irises of a user's eyes while the user inputs data field entries in low light environments via the screens.

Turning now to FIG. 38, one example of a chart review screen 3800 that may be rendered by a host device under control of an EMS charting application (e.g., the charting application 220 and/or the charting application 232 of FIGS. 2A and 2B), or a hospital's information system, is illustrated. As shown in FIG. 38, the chart review screen 3800 includes a demographic data control 3802, a document type selection control 3804, an attachment selection control 3806, a document selection control 3808, an attachment visualization control 3810, and a document visualization control 3812.

In some examples, the EMS charting application is configured to display, via the demographic data control 3802, demographic data of a patient, such as name, date of birth, gender, social security number, and address of the patient. The EMS charting application can be further configured to display, via the document type selection control 3804, a list of medical document types that are available for review via the EMS charting application. Examples of such documents include consent forms, EMS transport records, outcome documents, and documents related to medical procedural documentation pertinent to the patient. In some examples, the EMS charting application is also configured to receive user input selecting one or more of the document types displayed in the document type selection control 3804 and, in response to such input, display a list of medical documents of the selected type that are pertinent to the patient via the document selection control 3808. In the example illustrated within FIG. 38, the medical document type selected in the document type selection control 3804 is an EMS transport record type, and the list of medical documents displayed in the document selection control 3808 includes both a BLS transport record and an ALS transport record. The BLS transport record and/or the ALS transport record may include an ePCR.

In some examples, the EMS charting application is configured to receive user input selecting one or more of the documents displayed in the document selection control 3808 and, in response to such selection, display a visual representation of the selected document in the document visualization control 3812 and display a list of attachments to the selected document via the attachment selection control 3806. In the example illustrated within FIG. 38, the medical document selected in the document selection control 3808 is the ALS transport record and the list of attachments displayed in the attachment selection control 3806 includes a signature, 12-Lead EKG, and an insurance card of the patient.

In some examples, the EMS charting application is configured to receive user input selecting one or more of the attachments displayed in the attachment selection control 3806 and, in response to such selection, display a visual representation of the selected attachment in the attachment visualization control 3810. In the example illustrated within FIG. 38, the attachment selected in the attachment selection control 3806 is the signature of the patient.

Turning now to FIG. 39, one example of a chronology screen 3900 that may be rendered by a host device under control of an EMS charting application (e.g., the charting application 220 and/or the charting application 232 of FIGS. 2A and 2B) is illustrated. The chronology screen 3900 presents the patient encounter from initial call through hospital discharge as a synchronized timeline of data merged from the various systems used during the patient encounter. As shown in FIG. 39, the chronology screen 3900 includes a set of time entry control groups 3908 that each include a time entry control 3902, a source control 3904, and an owner control 3906. Each time entry control group 3908 specifies and is associated with an event within a timeline related to medical treatment of a patient. The information available in the chronology screen enables the EMS charting system to provide filtered search functions that enable a user to filter and/or sort the data collected in the EMS charting system for a patient across the various crews and agencies associated with a patient over multiple transitions in care. For example, a user may filter and/or sort between operational data (e.g., an arrival time, a departure time, an admission time, etc.) and clinical data (e.g., medication, 12 lead, vitals, etc.). The user may also sort and/or filter based on the source of the data, the intervention type, the time stamp, and/or the owner of the data.

In some examples, the EMS charting application is configured to display, via the set of time entry control groups 3908, timestamped event data that provides a holistic view of a patient's treatment. Examples of events that can be indicated by any of the time entry controls 3902 include receipt of an emergency call, dispatch of an EMS crew, response initiation by the EMS crew, arrival of the EMS crew on scene, recordation of patient vitals, and administration of interventions, such as medications, procedures, and the like. The EMS charting application can be further configured to display a source of the event data via the source controls 3904. Examples of event data sources include CAD systems (“CAD” in FIG. 39), navigation systems (“NAV” in FIG. 39), charting systems (“CRT” in FIG. 39), hospital information systems (“HIS” in FIG. 39), and medical devices, such as defibrillators (“DFB” in FIG. 39). The EMS charting application can be further configured to display an owner of the source of the event data via the owner controls 3906. Examples of owners include hospitals (“HOS” in FIG. 39) and agencies that employ BLS crews (“BLS” in FIG. 39) and ALS crews (“ALS” in FIG. 39), among others. In some examples, the EMS charting application is configured to receive user input selecting one or more of the source controls 3904 and/or the owner controls 3906 and to filter the set of time entry control groups 3908 to include only those members that are associated with (e.g., in the same row within the chronology screen 3900 as) the selected source controls 3904 and/or owner controls 3906.

### Example Synchronization Processes

As described above with reference to FIGS. 2A and 2B, in at least some examples of the EMS charting system, synchronization services (e.g., the synchronization services 222, 230, and 258) interoperate to ensure a common view of ePCR data is available to the cloud EMS charting applications (e.g., the cloud EMS charting application 232) and mobile EMS charting applications (e.g., the mobile EMS charting application 220) implemented within the EMS charting system, despite dynamic field conditions and system usage. In these examples, the synchronization services monitor local data stores (e.g., the charting data stores 223, 260, and 228) for changes. Further, in these examples, the synchronization services communicate changes to subscribed processes (e.g., other synchronization service instances) via synchronization messages specifying the changes. The synchronization services process the synchronization messages to maintain data stores local to the synchronization services.

In some situations, a synchronization service may encounter ePCR data fields that are in conflict. For example with reference to FIG. 2A, a first instance of the mobile EMS charting application hosted by the mobile computing device 212A may transmit a synchronization message specifying that the first name of the patient 210 is “John” to the synchronization service 258. Further a second instance of the mobile EMS charting application hosted by the mobile computing device 212B may transmit a synchronization message specifying that the first name of the patient 210 is “Joe” to the synchronization service 258. In this situation the synchronization service 258 is presented with ePCR data fields that are in conflict.

FIG. 34 illustrates a synchronization process 3400 executed by the synchronization services to adjudicate such conflicts. As shown in FIG. 34, the process 3400 starts with the synchronization service receiving 3402 values of a particular ePCR data field from multiple sources (e.g., from two or more instances of the synchronization service 222 hosted by different host devices). The values may be addressed to ePCR data fields that are static for the duration of the EMS call (e.g., patient name, date of birth, social security number, etc.) or dynamic within the EMS call (patient blood pressure, heart rate, etc.). In some examples, static data fields are identifiable within ePCR data through a data field identifier and dynamic data fields are identifiable with ePCR data through a data field identifier and a sample identifier (e.g., a timestamp).

Continuing with the process 3400, the synchronization service identifies 3404 a conflict in the values received in the operation 3402. For instance, in some examples, the synchronization service identifies a conflict where the values of the data fields are not equivalent. Alternatively or additionally, the synchronization service may identify a conflict if the values deviate by more than a configurable threshold value/distance that is specific to the data field.

Continuing with the process 3400, the synchronization service identifies 3406 an adjudication scheme to apply to the conflict. For instance, in some examples, the synchronization service identifies the adjudication scheme by locating an association between the data field identifier and an identifier of the adjudication scheme within a data structure storing configurable associations between such identifiers. The adjudication scheme identified can vary by data field. Some example adjudication schemes include first in wins, last in wins, majority vote wins (where 3 or more copies are received), authoritative source wins, and/or some combination of the above. Other example adjudication schemes include a machine learning model trained using data from previously documented EMS calls.

In some examples, the synchronization service determines the authoritative source based on the role of the originator of a copy of the data field. For instance, in some examples, the synchronization service adjudicates conflicts in preference of a highest-ranking role for the data field as defined within a data structure storing associations between identifiers of roles, identifiers of data fields, and ranks for the role with regard to the data field. For instance, in one example in which the synchronization service adjudicates conflicts using the authoritative source method, the synchronization service would recognize and store a copy of a data field originating from a user working as a paramedic over a copy of the data field originating from a user working as an AEMT and would recognize a copy of a data field originating from a user working as an AEMT over a copy of the data field originating from a user working as a EMT. Alternatively or additionally, the synchronization service may determine the authoritative source based on the domain of the data field in conflict. For instance, in one example, the synchronization service adjudicates conflicts regarding vital signs to a first EMT in charge of collection of vital signs, adjudicates conflicts regarding medications to a second EMT in charge of medications, and adjudicates conflicts regarding any data field value in favor of a paramedic. It should be noted that application of adjudication schemes can be configured by an EMS agency, in some examples. Furthermore, in an implementation, adjudication schemes can be configured to apply to particular data fields, particular types of EMS calls, particular shifts or crews, the agency overall, etc.

Continuing with the process 3400, the synchronization service executes 3408 the adjudication scheme, which generates a confidence metric indicative of the likelihood that the correct value of the data field was recognized and stored in the data field. It should be noted that, where the adjudication scheme considers timestamp values, the synchronization service may apply a time adjustment to some timestamps associated with one or more conflicting values.

Continuing with the process 3400, the synchronization service determines 3410 whether the confidence metric generated by the operation 3408 transgresses a configurable threshold value specific to the data field. If the confidence metric transgresses the threshold value, the process 3400 ends. If the confidence metric does not transgress the threshold value, the synchronization service requests 3412 user verification of the adjudicated data field and the process 3400 ends. The request may take the form of any human readable communication, such as a text message, email, in-app notification, and the like.

### Example Mobile Computing Device and Medical Devices

FIG. 35 illustrates one example of a mobile computing device 3500 that may be used to implement the mobile EMS charting application. In various examples, the mobile computing device 3500 is implemented using any of a variety of programmable devices (e.g., a device with data storage and at least one processor in data communication with the data storage). In some examples, the mobile computing device 3500 includes a plurality of interfaces 3504, one or more processors 3506, and a data storage device 3508 coupled to one another via a communication mechanism, such as a bus. In these examples, the mobile computing device 3500 also includes a battery 3510 to power the device and may include one or more antennas 3502. The data storage device 3508 can include a non-transitory data storage medium to store an operating system and one or more software applications or programs. These programs can include instructions executable by the one or more processors to implement the features disclosed herein and the methods that they execute. The plurality of interfaces in the mobile computing device 3500 include a user interface, a network interface configured to communicate with a network (e.g., the networks 206, 218 of FIG. 2A), and/or a medical device interface configured to exchange information with a medical device (e.g., one of the medical device(s) 214 of FIG. 2A). For instance, the plurality of interfaces 3504 may include a touchscreen, a camera, an NFC tag, and/or an RFID tag.

Particular examples of the mobile computing device 3500 include medical devices, wearable devices, smartphones, tablet computers, and laptop computers. Wearable devices that may serve as the mobile computing device 3500 include various garments with integrated technologies, watches, anklets, necklaces, belt buckles, and glasses.

In examples where the mobile computing device 3500 is implemented via a smartphone, a dedicated software application (i.e., the mobile EMS charting application) may be downloaded to the smartphone to facilitate the interactions described herein. For instance, the mobile EMS charting application may be written for an Android, iOS, Windows, or other operating system of the smartphone.

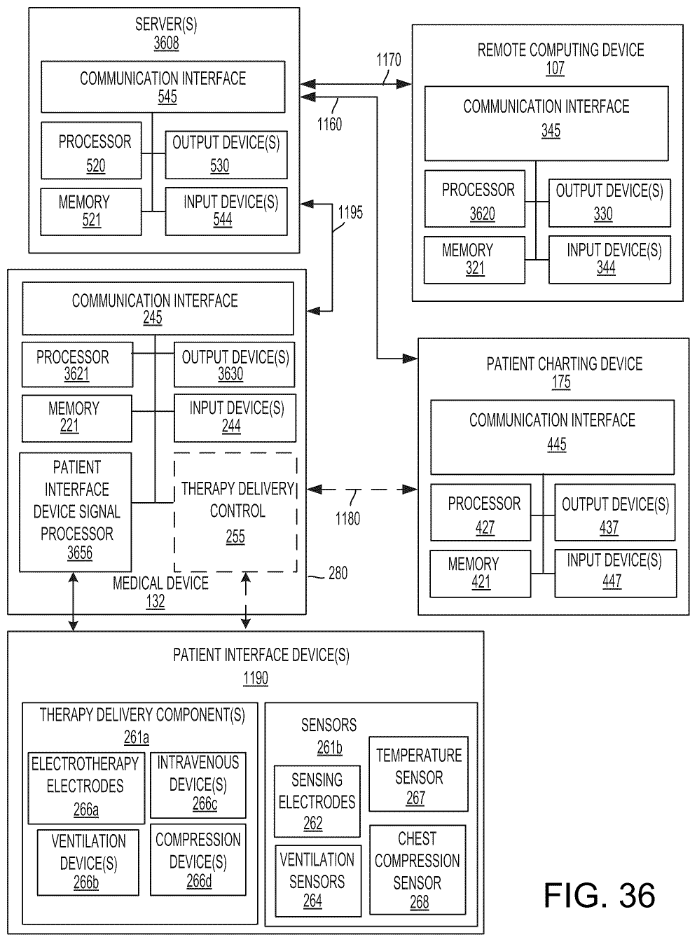

Referring to FIG. 36, a block diagram of examples of computing and medical device components are shown schematically.

The medical device 132 can include a processor 3621, a memory 221, one or more output devices 3630, one or more user input devices 244, and a communications interface 245. The communications interface 245 can include any of a variety of transmitters and/or receivers. For instance, in some examples, the communications interface 245 includes one or more of an NFC tag, an RFID tag, a barcode, and a QR code.

In various implementations, the medical devices 132 can include a defibrillator, patient monitor, defibrillator/monitor, an automated compression device, a therapeutic cooling device, an extracorporeal membrane oxygenation (ECMO) device, a ventilation device, combinations thereof, or another type of medical device configured to couple to one or more therapy delivery components to provide therapy to the patient. In an implementation, the medical devices 132 can include an integrated therapy delivery/monitoring device within a single housing 280. The single housing 280 can surround, at least in part, a patient interface device signal processor 3656 and/or a therapy delivery control 255.

The patient interface devices 1190 can include one or more therapy delivery component(s) 261a and/or one or more sensor device(s) 261b. The medical device 132 can be configured to couple to the one or more therapy delivery component(s) 261a. In combination, the medical device 132 and the one or more therapy delivery components can provide therapeutic treatment to a patient. In an implementation, the medical device 132 can include or incorporate the therapy delivery component(s) 261a. The therapy delivery component(s) 261a are configured to deliver therapy to the patient and can be configured to couple to the patient. For example, the therapy delivery component(s) 261a can include one or more of electrotherapy electrodes including defibrillation electrodes and/or pacing electrodes, chest compression devices (e.g., one or more belts or a piston), ventilation devices (e.g., a mask and/or tubes), drug delivery devices, etc. The medical device 132 can include the one or more therapy delivery component(s) 261a and/or can be configured to couple to the one or more therapy delivery component(s) 261a in order to provide medical therapy to the patient. The therapy delivery component(s) 261a can be configured to couple to the patient. For example, a care provider may attach the electrodes to the patient, and the medical device 132 (e.g., a defibrillator or defibrillator/patient monitor) may provide electrotherapy to the patient via the defibrillation electrodes. These examples are not limiting of the disclosure as other types of medical devices, therapy delivery components, sensors, and therapy are within the scope of the disclosure.

The medical devices 132 can include, for example, a therapeutic medical device capable of delivering a medical therapy. For example, the medical therapy can be electrical therapy (e.g. defibrillation, cardiac pacing, synchronized cardioversion, diaphragmatic or phrenic nerve stimulation) and the medical devices 132 can include a defibrillator, a defibrillator/monitor and/or another medical device configured to provide electrotherapy. As another example, the medical therapy can be chest compression therapy for treatment of cardiac arrest and the medical device 132 can be a mechanical chest compression device such as a belt-based chest compression device or a piston-based chest compression device. As other examples, the medical therapy can be ventilation therapy, therapeutic cooling or other temperature management, invasive hemodynamic support therapy (e.g. Extracorporeal Membrane Oxygenation (ECMO)), etc. and the medical device 132 can be a device configured to provide a respective therapy. In an implementation, the medical device 132 can be a combination of one or more of these examples. The therapeutic medical device can include patient monitoring capabilities via one or more sensors. These types of medical therapy and devices are examples only and not limiting of the disclosure.

The medical devices 132 can include, incorporate, and/or be configured to couple to the one or more sensor(s) 261b which can be configured to couple to the patient. The sensor(s) 261b are configured to provide signals indicative of sensor data to the medical device 132. The sensor(s) 261b can be configured to couple to the patient. For example, the sensor(s) 261b can include cardiac sensing electrodes, a chest compression sensor, and/or ventilation sensors. The one or more sensors 261b can generate signals indicative of physiological parameters of the patient. For example, the physiological parameters can include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. Additionally or alternatively, the one or more sensors 261b can generate signals indicative of chest compression parameters, ventilation parameters, drug delivery parameters, fluid delivery parameters, etc.

In addition to delivering therapy to the patient, the therapy delivery component(s) 261a can include, be coupled to, and/or function as sensors and provide signals indicative of sensor data (e.g., second sensor data) to the medical device 132. For example, the defibrillation electrodes can be configured as cardiac sensing electrodes as well as electrotherapy delivery devices and can provide signals indicative of transthoracic impedance, ECG, heart rate and/or other physiological parameters. As another example, a therapeutic cooling device can be an intravenous cooling device. Such a cooling device can include an intravenous (IV) device as a therapy delivery component configured to deliver cooling therapy and sense the patient's temperature. For example, the IV device can be a catheter that includes saline balloons configured to adjust the patient's temperature via circulation of temperature controlled saline solution. In addition, the catheter can include a temperature probe configured to sense the patient's temperature. As a further example, an IV device can provide therapy via drug delivery and/or fluid management. The IV device can also monitor and/or enabling monitoring of a patient via blood sampling and/or venous pressure monitoring (e.g., central venous pressure (CVP) monitoring).

The medical devices 132 can be configured to receive the sensor signals (e.g., from the therapy delivery component(s) 261a and/or the sensor(s) 261b) and to process the sensor signals to determine and collect the patient data. The patient data can include patient data which can characterize a status and/or condition of the patient (e.g., physiological data such as ECG, heart rate, respiration rate, temperature, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data can characterize the delivery of therapy (e.g., chest compression data such as compression depth, compression rate, etc.) and/or the patient data can characterize a status and/or condition of the medical equipment used to treat the patient (e.g., device data such as shock time, shock duration, attachment of electrodes, power-on, etc.).

In some examples, the components of 3621, 221, 3630, 244, 245, and 255 of the medical device 132 are communicatively coupled (directly and/or indirectly) to each other for bi-directional communication.

Although shown as separate entities in FIG. 36, the one or more of the components of the medical device 132 can be combined into one or more discrete components and/or can be part of the processor 3621. The processor 3621 and the memory 221 can include and/or be coupled to associated circuitry to perform the functions described herein.

In an implementation, the medical devices 132 can include a therapeutic medical device configured to deliver medical therapy to the patient. Thus, the medical devices 132 can optionally include the therapy delivery control module 255. For example, the therapy delivery control module 255 can be an electrotherapy delivery circuit that includes one or more capacitors configured to store electrical energy for a pacing pulse or a defibrillating pulse. The electrotherapy delivery circuit can further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 255 can be a compression device electro-mechanical controller configured to control a mechanical compression device. As a further example, the therapy delivery control module 255 can be an electro-mechanical controller configured to control drug delivery, temperature management, ventilation, and/or other type of therapy delivery. Alternatively, some examples of the medical devices 132 may not be configured to deliver medical therapy to the patient but can be configured to provide patient monitoring and/or diagnostic care. As shown in FIG. 36, in some examples, the therapy delivery control 255 exchanges messages 1180 with the charting device 175 (e.g., the patient charting device). These messages can include patient data descriptive of therapy provided to the patient or other patient data stored on the medical devices 132. This patient data can be used by an ePCR application in generating an ePCR documenting a dispatched EMS event.

The medical devices 132 can incorporate and/or be configured to couple to one or more patient interface devices 1190. The patient interface devices 1190 can include one or more therapy delivery component(s) 261a and one or more sensor(s) 261b. The one or more therapy delivery component(s) 261a and the one or more sensor(s) 261b sensor can provide one or more signals to the medical devices 132 via wired and/or wireless connection(s).

The one or more therapy delivery component(s) 261a can include electrotherapy electrodes (e.g., the electrotherapy electrodes 266a), ventilation device(s) (e.g., the ventilation devices 266b), intravenous device(s) (e.g., the intravenous devices 266c), compression device(s) (e.g., the compression devices 266d), etc. For example, the electrotherapy electrodes can include defibrillation electrodes, pacing electrodes, and/or combinations thereof. The ventilation devices can include a tube, a mask, an abdominal and/or chest compressor (e.g., a belt, a cuirass, etc.), etc. and combinations thereof. The intravenous devices can include drug delivery devices, fluid delivery devices, and combinations thereof. The compression devices can include mechanical compression devices such as abdominal compressors, chest compressors, belts, pistons, and combinations thereof. In various implementations, the therapy delivery component(s) 261a can be configured to provide sensor data and/or be coupled to and/or incorporate sensors. For example, the electrotherapy electrodes can provide sensor data such as transthoracic impedance, ECG, heart rate, etc. Further the electrotherapy electrodes can include and or be coupled to a chest compression sensor. As another example, the ventilation devices can be coupled to and/or incorporate flow sensors, gas species sensors (e.g., oxygen sensor, carbon dioxide sensor, etc.), etc. As a further example, the intravenous devices can be coupled to and/or incorporate temperature sensors, flow sensors, blood pressure sensors, etc. As yet another example, the compression devices can be coupled to and/or incorporate chest compression sensors, patient position sensors, etc. The therapy delivery control module 255 can be configured to couple to and control the therapy delivery component(s) 261a.

In various implementations, the sensor(s) 261b can include one or more sensor devices configured to provide sensor data that includes, for example, but not limited to ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, images and/or videos via ultrasound, laryngoscopy, and/or other medical imaging techniques, near-infrared reflectance spectroscopy, pneumography, cardiography, and/or patient movement. Images and/or videos can be two-dimensional or three-dimensional.

The sensor(s) 261b can include sensing electrodes (e.g., the sensing electrodes 262), ventilation sensors (e.g., the ventilation sensors 264), temperature sensors (e.g., the temperature sensor 267), chest compression sensors (e.g., the chest compression sensor 268), etc. For example, the sensing electrodes can include cardiac sensing electrodes. The cardiac sensing electrodes can be conductive and/or capacitive electrodes configured to measure changes in a patient's electrophysiology, for example to measure the patient's ECG information. In an implementation, the sensing electrodes can be configured to measure the transthoracic impedance and/or a heart rate of the patient. The ventilation sensors can include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, and combinations thereof. The temperature sensors can include an infrared thermometer, a contact thermometer, a remote thermometer, a liquid crystal thermometer, a thermocouple, a thermistor, etc. and can measure patient temperature internally and/or externally. The chest compression sensor can include one or more motion sensors including, for example, one or more accelerometers, one or more force sensors, one or more magnetic sensors, one or more velocity sensors, one or more displacement sensors, etc. The chest compression sensor can be, for example, but not limited to, a compression puck, a smartphone, a hand-held device, a wearable device, etc. The chest compression sensor can be configured to detect chest motion imparted by a rescuer and/or an automated chest compression device (e.g., a belt system, a piston system, etc.). The chest compression sensor can provide signals indicative of chest compression data including displacement data, velocity data, release velocity data, acceleration data, compression rate data, dwell time data, hold time data, blood flow data, blood pressure data, etc. In an implementation, the sensing electrodes and/or the electrotherapy electrodes can include or be configured to couple to the chest compression sensor.

Continuing with FIG. 36, examples of components of the charting device 175 are shown schematically. In an implementation, the charting device 175 can be configured as a computing device. The charting device 175 can include a processor 427, a memory 421, one or more output devices 437, one or more user input devices 447, and a communications interface 445. FIG. 36 also illustrates schematic examples of components of the computing device 107. As shown in FIG. 36, the computing device 107 can include a processor 3620, a memory 321, one or more output devices 330, one or more user input devices 344, and a communications interface 345. FIG. 36 further illustrates schematic examples of components of the server(s) 3608. As shown in FIG. 36, the server(s) 3608 can include a processor 520, a memory 521, one or more output devices 530, one or more user input devices 544, and a communications interface 545.

Each of the charting device 175 (e.g., the charting device) and the computing device 107 can be a computer system, such as a desktop, notebook, mobile, portable, or other type of computing system. Each of these devices 175 and 107 can include server(s) and/or access server(s) via a monitor and/or other connected user interface device. Although described as server(s), the server(s) 3608 can be another type of computing system including for example a desktop, notebook, mobile, portable, or other type of computing system.

As shown in FIG. 36, each of the devices 175 and 107, along with the server(s) 3608 and the medical devices 132, includes a bus or other interconnection mechanism that communicably couples the processor, memory, output devices, input devices, and communication interface included therein. The bus can include a PCI/PCI-X or SCSI based system bus depending on the storage devices used, for example.

The processors 3621, 3620, 427, and 520 can each include a processor, such as, but not limited to, an Intel® Itanium® or Itanium 2® processor(s), or AMD® Opteron® or Athlon MP® processor(s), or any of a Motorola® line of processors. The communication interfaces 245, 345, 445, and 545 can each be any of an RS-232 port for use with a modem-based dialup connection, a 10/100 Ethernet port, or a Gigabit port using copper or fiber, for example. The communication interfaces 245, 345, 445, and 545 may be chosen depending on a network(s) such a Local Area Network (LAN), Wide Area Network (WAN), or any network to which the medical devices 132, the charting device 175, the computing device 107, and/or the server(s) 3608 may connect. The memories 221, 321, 421, and 521 can be Random Access Memory (RAM), Read Only Memory (ROM), Flash memory, and/or another dynamic volatile and/or non-volatile storage device(s). The memories 221, 321, 421, and 521 can be used to store information and instructions. For example, hard disks such as the Adaptec® family of SCSI drives, an optical disc, an array of disks such as RAID (e.g. the Adaptec family of RAID drives), or any other mass storage devices may be used. The components described above are meant to exemplify some types of possibilities. In no way should the aforementioned examples limit the scope of the disclosure. The memories 221, 321, 421, and 521 can further include removable storage media such as external hard-drives, floppy drives, flash drives, IOMEGA® Zip Drives, Compact Disc-Read Only Memory (CD-ROM), Compact Disc-Re-Writable (CD-RW), or Digital Video Disk-Read Only Memory (DVD-ROM), for example.

Continuing with FIG. 36, the server(s) 3608 can include, for example, the one or more storage server(s) and one or more application server(s). In some examples, the server(s) 3608 are configured to exchange messages 1170 with the computing device 107. These messages can include charting and/or case data as described above. In some examples, the server(s) 3608 are configured to exchange messages 1160 with the charting device 175 via an ePCR API. These messages can include data descriptive of ePCRs generated by the charting device 175. In some examples, the server(s) 3608 are configured to exchange messages 1195 with the medical devices 132. These messages can include data descriptive of a patient being treated via the medical device and/or treatment being delivered by the medical devices 132.

Some examples of the present disclosure include various steps, some of which can be performed by hardware components or can be embodied in machine-executable instructions. These machine-executable instructions can be stored on a non-transitory data storage medium and can be used to cause a general-purpose or a special-purpose processor programmed with the instructions to perform the steps. The non-transitory data storage medium can further store an operating system and the machine-executable instructions can be included within one or more software applications or programs, such as the ePCR application. These programs can implement the features disclosed herein and the methods that they execute. Alternatively, the steps can be performed by a combination of hardware, software, and/or firmware, on one device and/or distributed across multiple devices and/or processors. In addition, some examples of the present disclosure can be performed or implemented, at least in part (e.g., one or more modules), on one or more computer systems, mainframes (e.g., IBM mainframes such as the IBM zSeries, Unisys ClearPath Mainframes, HP Integrity NonStop server(s), NEC Express series, and others), or client-server type systems. In addition, specific hardware aspects of examples of the present disclosure can incorporate one or more of these systems, or portions thereof.

FIG. 37A illustrates an example of a logical and physical architecture of a mobile EMS charting application 220 as part of a SaaS platform. In an implementation, the mobile EMS charting application 220 executing on the mobile device 212A, for example, a smartphone in a mobile EMS environment 3704, may communicatively couple to a charting system server 3718. The mobile EMS environment 3704 may include a navigation device 436 that is configured to communicatively couple to a navigation system server 3728, a CAD system server 3730, and one or more global positioning systems and/or cellular positioning systems. The positioning system 3740 may use global positioning (e.g., satellite positioning) and/or cellular positioning data to locate the navigation device 436. The mobile EMS charting application 220 may use the positioning data to determine a context for the mobile device 212A. The mobile EMS environment 3704 may further include one or more medical device(s) 3732. In various implementations, the medical device(s) 3732 can include a patient treatment device, or another kind of device that includes patient monitoring and/or patient treatment capabilities, according to examples of the present disclosure. For example, the medical device(s) 3732 can include a defibrillator and can be configured to deliver therapeutic electric shocks to the patient. In some examples, the medical device(s) 3732 can deliver other types of treatments, such as ventilation, operating a respirator, and/or administering drugs or other medication. The mobile EMS environment 3704 may also include an emergency vehicle, such as an ambulance, a fire engine, an EMS crew transport vehicle, and/or a helicopter.

In such an implementation, the mobile EMS charting application 220 may receive and utilize data from other elements of an EMS SaaS platform 3726 executing in a cloud environment 3702. The SaaS platform 3726 may implement one or more services via inclusion of one or more CAD system server(s) 3730, one or more navigation system server(s) 3728, one or more patient charting system server(s) 3722, one or more medical billing system server(s) 3767, a medical device case data store 3724, a charting system data store 3720, one or more integration platform server(s) 498, and one or more data analytics system server(s) 952. Although all of the CAD system server 3730, the navigation system server(s) 3728, the patient charting system server(s) 3722, the medical billing system server(s) 3767, a medical device case data store 3724, a charting system data store 3720, the integration platform server(s) 497, and the data analytics system server(s) 952 are illustrated as being components of the SaaS platform 3726 for simplicity, additionally or alternatively some or all of these may be part of separate SaaS platforms and the example of FIG. 37A is not limiting of the disclosure. In some implementations, the elements of the SaaS platform in FIG. 37A may exist as two or more separate elements with at least one being the SaaS platform 3726 and at least one being separate from the SaaS platform 3726. For example, a first company and a second company may provide first medical billing services and second medical billing services, respectively. The first company may also provide a charting system and enable interoperation between the first medical billing services and the charting system via a single SaaS platform. However the charting system may also be communicatively coupled to and able to interoperate with the second medical billing services offered outside of the SaaS platform hosting the charting system. This example may similarly apply to the other elements of the SaaS platform 3726 as shown in FIG. 37A. In an implementation, entities within a common SaaS platform 3726 may share a centralized data storage. For example, if a first company provides a CAD system, a navigation system, a medical billing system, a charting system, a data analytics system, and medical devices, then all of these systems may have access to a central and common data store associated with the SaaS platform 3726. The central and common data store may be a cloud-based storage associated with a single business entity or with an account of a single business entity. In an implementation, the SaaS platform 3726 enables sharing of information (e.g., patient data) between entities of the platform and enables the mobile EMS charting application 220 to enhance patient care through advanced caregiver guidance and recordation based on this sharing. However, if, for example, a second company provides a charting system, that second company may access services from the SaaS platform 3726, but the second company's charting system data store may be separate from that of the SaaS platform 3726. In some examples, the SaaS platform 3726 is configured to enable sharing of the information via its inclusion of an integration platform (e.g., the integration platform 497 described further below with reference to FIG. 37C) that is made up of the integration platform server(s) 498. For example, the integration platform server(s) 498 may be configured to expose and implement an API that passes information from one SaaS platform entity to another without accessing the information itself, thereby ensuring security, private communications between SaaS platform members. The integration platform 497 may also provide data formatting translation services that enable interoperability between services provided by different business entities. Thus, CAD services, navigation services, medical billing services, charting services, medical records, data analytics, and/or medical devices from a first business entity may exchange information with CAD services, navigation services, medical billing services, charting services, medical records, data analytics, and/or medical devices from a second business entity even where the two business entities follow different data structures or formats. For example, initiation of a call by the CAD 3730 and communication, via the integration platform 497, to the mobile EMS charting application 220 of the initiated call may enable the mobile EMS charting application 220 to query, again via the integration platform 497, a medical record repository 3705. The mobile EMS charting application 220 may store query results in the ePCR and/or generate caregiver prompts based on the query result. Further, the mobile EMS charting application 220 may provide ePCR data to the charting system server 3718 and/or the billing system server 3767, via the integration platform 497. Alternatively, via interoperation with the integration platform 497, the CAD 3730 may communicate with the charting system 3718 and the charting system 3718 may then communicate with the medical record repository 3705 and provide query results to the mobile EMS charting application 220. For instance, the charting system 3718 may receive demographic data (a driver's license number or other patient reference code) from the EMS charting application 220 (e.g., via a scanned driver's license or other patient identification documentation) and may use the received demographic data to query, via the integration platform 497, the other elements of the SaaS platform 3726 for additional demographic data, medical history, allergies, medications, and/or insurance information of the patient. Results to these queries returned to the EMS charting application 220 can be accessed by users via screens such as those described above (e.g., with reference to FIG. 12, among others). For example, via interoperation with the integration platform 497, the medical billing system 3767 may receive and/or provide charting information and/or patient care information (e.g., based on a medical history provided by billing records) to the mobile EMS charting application 220 during or after the medical event via communications with the charting system server 3718. In some implementations, the patient reference code is created by the SaaS platform 3726 or by the mobile EMS charting application 220. This patient reference code is shared with all of the processes implemented within the SaaS platform 3726 to provide for an internal reference that can be used to mark and track the patient's information within the SaaS platform 3726. In an implementation, the charting system 3718 may interface with the data analytics system server(s) 952 to utilize the capabilities of a data analytics platform (e.g., as described in regard to FIGS. 37A and 37B).

As shown in FIG. 37A, the cloud environment 3702 may be implemented within a data center or other high capacity computing facility with high speed internet connectivity. The platform 3726 may include a plurality of dedicated servers (e.g., a farm or cluster of computer systems) within the data center that are interconnected via a high speed, private network. Each of the servers illustrated within the platform 3726 may be one or more physical and/or one or more virtual servers. The servers can include one or more application servers, web servers, and/or database servers. The servers can include enterprise servers configured to support an organization as a single tenant and/or cloud servers configured to support multiple organizations as multiple tenants.

It should be noted that the software applications hosted by servers within the platform 3726 are configured to expose APIs that enable the software applications to communicate with one another. These APIs are configured to receive, process, and respond to commands issued by software applications hosted on the same server or a different server in the platform. For instance, these APIs enable any of the servers in the platform 3726 to transmit queries, information, patient reference codes etc. and otherwise communicate with one or more other servers in the platform 3726 and/or with the mobile EMS charting application 220.

The APIs may be implemented using a variety of interoperability standards and architectural styles. For instance, in one example, the APIs are web services interfaces implemented using a REST architectural style. In this example, the APIs communicate with a client process using HTTP along with JavaScript Object Notation and/or extensible markup language. In some examples, portions of the HTTP communications can be encrypted to increase security. Alternatively or additionally, in some examples, the APIs are implemented as a NET web API that responds to HTTP posts to particular uniform resource locators. Alternatively or additionally, in some examples, the APIs are implemented using simple file transfer protocol commands and/or a proprietary application protocol accessible via a transmission control protocol socket. Thus, the APIs described herein are not limited to a particular implementation.

The network within the cloud environment 3702 and the local network with the mobile EMS environment 3704 can include one or more communication networks through which the computing devices within these environments send, receive, and/or exchange data. In various implementations, the network can include a cellular communication network and/or a computer network. In some examples, the network includes and supports wireless network and/or wired connections. For instance, in these examples, the network may support one or more networking standards such as GSM, CDMA, USB, BLUETOOTH, CAN, ZIGBEE, Wireless Ethernet, Ethernet, and TCP/IP, among others. The network may include both private networks, such as LANs, and public networks, such as the Internet. It should be noted that, in some examples, the network may include one or more intermediate devices involved in the routing of packets from one endpoint to another. However, in other examples, the network can involve only two endpoints that each have a network connection directly with the other.

The data store 3720 may be implemented by, for example, a database (e.g., a relational database) and stored on a non-transitory storage medium. The data store 3720 is configured to store ePCRs generated by the mobile EMS charting application 220. It should be noted that ePCR data stored in the data store 3720 may be used to populate ePCRs subsequently generated by instances of the charting application 220 via API interoperations between the charting application 220 and the charting system server 3718.

In some examples, the charting system server 3718 is configured to interoperate via the integration platform 497 with one or more of the CAD system server 3730, the navigation system server 3728, the billing system server 3767, the case data store 3724, the data analytics system server 952, the hospital system 3772 and/or the medical record repository 3705, and/or multiple instances of the mobile EMS charting application 220 to acquire charting information. The charting information may include dispatch records, patient identification data, medical records for patients, and/or other information available from these interoperable servers and systems. Although all of the CAD system server 3730, the navigation system server 3728, the billing system server 3767, the case data store 3724, the data analytics system server 952, the hospital system 3772 and/or medical record repository 3705 are illustrated as being interoperable through the integration platform 497 for simplicity, additionally or alternatively some or all of these may be communicatively coupled and interoperable via other information exchange services and systems and the example of FIG. 37A is not limiting of the disclosure. It should be noted that, in some examples, the charting system server 3718 is configured to periodically update medical records by interoperating with the other servers in the platform 3726 and/or devices within the mobile EMS environment 3704. For instance, in one example, the charting system server 3718 periodically requests updated billing codes from the billing system server 3767 and updates medical records stored in the data store 3720 accordingly. These billing codes are a source of information for previous medical treatments. For instance, billing codes can indicate that a patient received treatment for asthma, treatment for cardiac arrest, treatment for a drug overdose, prescription information, and/or recent surgeries. This information may be clinically actionable and relevant. For example, stitches from recent surgeries could reopen. Devices implanted during surgery may need to be addressed. Treatments for drug overdose may indicate a need to avoid opioids. Repeated treatments and prescriptions could indicate chronic conditions and/or contraindications. Moreover, the interval between periodic updates can be adjusted based on the level of interaction between a patient and the systems described herein. For example, where a patient is actively involved in an EMS encounter, the interval can be decreased to minutes or even seconds. This can allow, for example, for an electrocardiogram (EKG) to be imported to, for instance, the charting system server 3718 and for the charting system server 3718 to relay the EKG to the mobile device 212A for display by the mobile EMS charting application 220.

The CAD system server 3730 may receive requests to record calls from a public safety answering point 411 and process the requests to generate and store call records. The CAD system server 3730 may transmit dispatch requests to an EMS agency 412 to dispatch EMS personnel (e.g., the EMS caregivers 208A-208N of FIG. 2A) to service calls. The CAD system server 3730 may transmit addresses to call locations to the mobile EMS charting application 220 so that the mobile EMS charting application 220 can acquire routes to call locations by interoperating with the positioning system 3740 and/or the navigation system server 3728. In an implementation, the mobile EMS charting application 220 may provide real time, step by step directions to call locations via the routes.

The case data store 3724 receives case files uploaded by the medical devices 3732. The case data store 3724 can be implemented by, for example, a database (e.g., a relational database) and stored on a non-transitory storage medium. In an implementation, the case data store 3724 includes a plurality of records that store case data derived from case files from a plurality of medical devices used to treat patients during encounters. Moreover, in some examples, the case data store 3724 can store complete copies of the case files themselves (e.g., as large binary objects). The case data stored in the case data store 3724 can document patient encounters from the point of view of medical devices. As such, case data generated by a medical device during a patient encounter can include an identifier of the medical device, physiologic parameter values of the patient recorded by the medical device during the encounter, characteristics of treatment provided by the medical device to a patient during the encounter, actions taken by care providers during the encounter, and timestamps associated with medical device case data. For instance, where the medical device is a defibrillator, the case data can include patient physiological parameters such as ECG data for the patient, as well as characteristics of therapeutic shocks delivered by the defibrillator to the patient, CPR performance data, and timestamps reflecting a power-on time for the defibrillator and associated with recorded case data, among other information. The mobile EMS charting application 220 may receive case data from the medical device(s) 3732 via the charting system server 3718 and/or via short-range communications with the medical device(s) 3732.

The data stores 3720 and 3724 can be organized according to a variety of physical and/or logical structures. In at least one example, the data stores 3720 and 3724 are implemented within a relational database having a highly normalized schema and accessible via a structured query language (SQL) engine, such as ORACLE or SQL-SERVER. This schema can, in some implementations, include columns and data that enable the data stores 3720 and 3724 to house data for multiple tenants. In addition, although the description provided above illustrates the data stores 3720 and 3724 as relational databases, the examples described herein are not limited to that particular physical form. Other databases may include flat files maintained by an operating system and including serialized, proprietary data structures, hierarchical database, xml files, NoSQL databases, document-oriented databases and the like. Thus, the data stores 3720 and 3724 as described herein are not limited to a particular implementation.

Continuing with FIG. 37A, the billing system server 3767 implements a medical billing system. The billing system server 3767 can store patient identification data, information regarding claims involving patients, payments status of the claims, and the like. The patient identification data stored in the billing system server 3767 can include, for example, patient provider and insurance information.

Continuing with FIG. 37A, the data analytics system server 952 may perform derive insights from data accessed from a variety of disparate data sources 3770 such as insurance companies, credit records, medical records, etc. These data sources may provide, for example, patient financial data, medical payer data, credit score data, patient demographic data, historic medical claims data, medical provider data, etc. The data analytics system server 952 may apply machine learning analysis and or other statistic data analysis techniques to identify patterns in the various data records to produce complex predictive intelligence, for example by deriving insights, metrics and/or calculating estimates using the data from the disparate data sources. The output of the data analytics system server 952 may include predictive intelligence regarding remittance value for medical services from individual payers or for combined payers (e.g., primary insurance plus secondary insurance, primary insurance plus patient deductible value, etc.). This information may include predicted payment schedules, prior authorization information or deductible information. In some implementations, the data analytics system server 952 may provide demographic verification and insurance discovery tools. The system server 952 may provide predictive intelligence output in various formats, including stored data files or data rendered in a graphical user interface in the form of short textual and/or numerical answers, charts, graphs, interactive spreadsheets, and/or interactive reports, in some examples.

Continuing with FIG. 37A, one or more hospital system(s) 946 originate admin records for sharing with the integration platform 497. The hospital system(s) 946, for example, may obtain medical information regarding a patient from a medical records repository 3705 via the integration platform 497 and add the patient information (e.g., demographics information, etc.) into a new admin record.

Interoperations facilitated by the integration platform 497 between the mobile EMS charting application 220 and the various elements of the SaaS platform 3726 may enable the mobile EMS charting application 220 to provide various types of information relevant to the patient care and the EMS interaction as shown in Table 3. The information in Table 3 is exemplary and is not limiting of the disclosure. These examples are of queries from the EMS caregiver that the mobile EMS charting application 220 may recognize and respond to via API interoperations with one or more of the CAD system server 3730, the navigation system server 3728, the billing system server 3767, the charting system server 3718, the medical record repository 3705, the charting data store 3720, the case data store 3724, hospital system(s) 3772, data sources 3770, and the data analytics system server 952. The API interoperations with the billing system server 3767, the medical record repository 3705, the charting data store 3720, and the case data store 3724 may occur via the integration platform 497.

TABLE 3

| Category | Examples |
|---|---|
| Historical Patient Care | Previous transports of patient by agency |
| | Previous risks and/or complications for |
| | the patient |
| | Progressive information as patient call |
| | evolves |
| Predictive patient care | Current level of service |
| analysis | |
| | Level of care data points |
| | ICD coding suggestions based on |
| | assessments, protocols, medications, |
| | airway, and/or procedures |
| Patient care reimbursement | Self-pay patient |
| analysis | |
| | Confirmation of health insurance |
| | Preferred providers |
| | Necessary pre-authorizations |
| Patient care knowledge base | Current medication interactions |
| | Passive protocols for patient |
| | Active protocols for patient |
| | Next step in a protocol (e.g., trauma, |
| | sepsis, cardiac, respiratory, etc.) |
| Patient Coordination of Care | Coordinate care with destination |
| with Dispatch | |
| | Send current patient report to hospital |
| | while patient is en route |
| | Send historical patient report to hospital |
| Dispatch and Navigation | Find closest medical facility for medical |
| | care needed by patient based on |
| | provided EMS care |
| | Find best location for transport vehicle at |
| | the emergency scene |
| | Schedule medical procedure and |
| | transport for a future date (e.g., record |
| | and provide basic information and |
| | specialty care transport interactive |
| | dialog) |
| Data Exchange and analytics | Previous patient hospital outcomes |
| | Crew level of service and vehicle |
| | allocation for particular time period |
| | Analysis of destinations filtered by |
| | patient care requirements |
| Billing | Open an account, provide insurance |
| | information, confirm patient |
| | demographics, claims checklist, medical |
| | necessity analysis, closest facility analysis, |
| | insurance eligibility analysis, billing |
| | worklists, billing rules |

In combination, the systems illustrated in FIG. 37A can produce accurate and comprehensive documentation that improves continuity of patient care and overall patient health outcomes. More specifically, continuity of care may benefit from a record that thoroughly describes symptoms, physiological metrics, and treatments provided.

FIG. 37B illustrates an example of a logical and physical architecture of data analytics platform 951 as part of the SaaS platform 3726. The data analytics platform 951, for example, may gather and/or network the various information sources, in some examples, from one or more claims data sources 4006, benefits information sources 4008, and/or service providers 4012. The data analytics platform 951 may generate, through analyzing the gathered and/or networked information using machine learning classifiers and/or other clustering and pattern identification algorithms, complex predictive intelligence for a set of clients 4004. The clients 4004 may access the data analytics platform 951 via a networked connection. The data analytics platform 951, in some implementations, includes a demographic verification engine 4014 for verifying and/or supplementing patient information with demographic information or other patient record information obtained from an external source. The demographic verification engine 4014, for example, may compare external information with patient data 4040 maintained by the medical provider and update or supplement the information where appropriate.

In some implementations, the data analytics platform 951 includes a coverage verification engine 4016 configured to automatically submit patient information to a payer system to confirm active coverage of the patient by the payer. The coverage verification engine 4016, for example, may be invoked where the coverage for the patient is uncertain or where the most recent coverage verification for the patient was obtained outside a threshold window of time such as, in some examples, over one month, over three months, or longer than six months. In some embodiments, the coverage verification engine 4016 coordinates with the demographic verification engine 4014 to update and/or supplement demographic information related to a patient.

In some implementations, the data analytics platform 951 includes a payer identification engine 4018 for automatically matching one or more payers with a patient based on demographic and/or other known information regarding the patient. The payers, for example, may include primary insurance providers, Medicare, and Medicaid. Further, the payers may include liability payers such as automotive liability insurance, homeowner insurance, worker compensation insurance, and/or business liability insurance. In some implementations, the payer identification engine 4018 automatically confirms coverage by calling the coverage verification engine 4016 for each likely payer candidate. Upon confirming, in some implementations, the response from a particular payer may identify one or more secondary payers. For example, when verifying coverage of a primary payer, the primary payer may confirm active coverage and further identify one or more secondary insurance sources on record as being available to the patient.

In some implementations, the data analytics platform 951 includes a payer preference identification engine 4020 for analyzing available payers in view of services and/or products that will be submitted for reimbursement to identify the most appropriate and/or most desirable payers for claim submission. The data analytics platform 951, in some implementations, includes a payer payment pattern engine 4022 configured to analyze historic reimbursement data for a payer to identify trends or patterns in amounts, timing, and/or denials.

In some implementations, the data analytics platform 951 includes a patient payment pattern engine 4024 to identify patient payment trends from historic claims (e.g., claims data 4056 cross referenced with patient data 4040) based upon a patient medical debt score or rating. In some embodiments, the patient payment pattern engine 4024 further uses patient demographic data or other information to refine analysis of similar patients. The patient payment pattern engine 4024 identifies patient payment outcomes related to patients similar to the payer at least in medical credit score or rating.

In some implementations, the data analytics platform 951 includes a payment pattern application engine 4026 configured to apply payer payment patterns produced by the payer payment pattern engine 4022 and/or patient payer patterns produced by the patient payment pattern engine 4024 to calculate payment estimations. Further, in some embodiments, the payment pattern application engine 4026 determines a confidence level or rating associated with the payment estimate. The data analytics platform 951, in some implementations, includes a patient likelihood to pay analysis engine 4028 for determining whether a remaining balance is likely to be repaid by the patient. The patient likelihood to pay analysis engine 4028, in some embodiments, requests a patient financial clearance or risk analysis related to medical debt from a credit reporting agency specializing in healthcare reimbursement data, such as TransUnion LLC of Chicago, Illinois or Experian Health by Experian Information Solutions, Inc. of Costa Mesa, CA. In some implementations, the patient likelihood to pay analysis engine 4028 analyzes historic patient payment trends identified by the patient payment patterns engine. The data analytics platform 951 determines a likelihood of the remaining balance being repaid by the patient based on the patient financial clearance or risk analysis and, in some embodiments, the historic payment trends.

The data analytics platform 951, in some implementations, includes a patient matching engine 4030 for identifying similar patients based upon patient demographic data and/or other information relevant to a patient, such as, in some examples, a medical debt score or rating, one or more services provided to the patient, and/or provider plan coverage. The patient matching engine 4030 may access the patient data 4040 to match features to insured persons in the data repository 4010. The data analytics platform 951, in some implementations, includes a payer pre-approval analysis engine 4032 for determining likelihood of the payer requiring pre-approval for one or more services and/or products recommended by or scheduled for performance by a medical provider. The payer pre-approval analysis engine, for example, may analyze, for example using the payer payment pattern engine 4022, trends in historic claims data 4056 corresponding to a payer of the patient's coverage to identify claims rejections related to one or more products and/or services identified to the payer pre-approval analysis engine 4032.

The data analytics platform 951, in some implementations, includes a payer pre-approval request engine 4034 for automatically submitting a pre-authorization request in relation to a scheduled service or recommended product. In various implementations, the platform 951 may automatically issue a request for authorization from the payer, may automatically notify a third party to request authorization from the payer, or may provide a notice to the user of the platform 951 that the prior authorization is required. The notice to the user may include an identification of the third party to request this authorization.

In some implementations, the data analytics platform 951 includes a liability applicability analysis engine 4035 configured to analyze information related to a medical claim to identify whether liability insurance is likely to apply. The data analytics platform 951, in some implementations, includes a patient information updating engine 4036 for expanding upon patient demographic data that may be insufficient for uniquely identifying the patient and/or that contains ambiguous or contradictory information on comparison to another trustworthy source of patient information.

In some implementations, the data analytics platform 951 includes an assistance availability analysis engine 4038 configured to automatically investigate potential additional sources of medical debt coverage for uninsured or underinsured patients. The assistance availability analysis engine 4038, for example, may analyze patient demographic data to identify additional funding sources such as, in some examples, any federal programs, state programs, charitable foundation programs, and/or foundation discount programs. The additional funding sources may vary by location, type of service provider, income level, age, and/or employment status of the patient. Upon identifying eligibility for at least certain programs, such as the federal Medicare and Medicaid programs, the assistance availability analysis engine 4038 may automatically initiate enrollment in the programs by filling out online form(s) or other eligibility paperwork using the demographic data stored by the data analytics platform 951 in the patient data 4040. The data analytics platform 951, in some implementations, includes a payment trends analysis engine 4039 configured to analyze remittance received from payers and/or patients to identify patterns within the payments. The patterns, in some examples, may include a length of time between claim submission and reimbursement, a relative amount paid compared to amount billed, patterns of payer rejections, and patterns of claims re-filings directed to other sources of payments, such as liability payers.

In some implementations, the data analytics platform 951 includes the projected revenue analysis engine 4070 configured to analyze historical payment patterns, for example obtained from the payment trends analysis engine 4039, in view of recent claims to estimate revenue metrics 4064. The revenue metrics 4064, in some examples, may include remittance by payer, remittance by upcoming revenue cycle, remittance by service type (e.g., billing codes category), and/or remittance by location. In some implementations, the data analytics platform 951 includes a procedure trends analysis engine 4072 configured to analyze historic claims data for patients who underwent major procedures, such as surgical procedures, to identify patterns within the claims data near the time of the procedure and shortly thereafter (e.g., days, 1 week, weeks, 1 month, or up to multiple months) that may be indicative of follow-on services and/or prescription products commonly associated with the major procedure. The patterns, in some examples, may include services commonly paired with each major procedure, common follow-on services to each major procedure, and/or prescriptions commonly paired with each major procedure.

In some implementations, the data analytics platform 951 includes the projected procedure analysis engine 4074 configured to analyze historical major procedure service pairing patterns, for example obtained from the procedure trends analysis engine 4072, in view of recent claims to estimate projected additional claims. In some embodiments, the claims projections are automatically analyzed to better anticipate ordering needs for medical equipment and other products associated with the projected services, staffing needs for the projected services, and/or potential mitigation options to better support the patient in avoiding certain follow-on claims, such as sepsis.

In some implementations, the data analytics platform 951 includes the report generation engine 4078 configured to organize historic and/or forecast metrics such as revenue metrics 4064 and remittance metrics 4066 and prepare presentations related to the metrics.

In some implementations, the data analytics platform 951 includes a coverage coordination analysis engine 4076 for coordinating benefits coverage between multiple payers available to the patient. The coverage coordination analysis engine 4076, for example, may coordinate benefits based on a legal or administrative hierarchy, such as the coordination of benefits (COB) responsibilities published by the United States Centers for Medicare & Medicaid Services (CMS), the Coordination of Benefits Model Regulation by the National Association of Insurance Commissioners (NAIC), and/or U.S. statutes directed to federal employees health benefits regulations.

FIG. 37C illustrates an example of a logical and physical architecture of integration platform 497. In an implementation, the integration platform may be part of the SaaS platform 3726. In some implementations, an integration platform 497 may be configured to exchange data between the various processes implemented within the SaaS platform 3726 and external systems using a single standard data format. The single standard data format may be a common data format recognized or used by one or more entities of the cloud environment and/or the SaaS platform. The integration platform 497 may translate or convert data that is stored in, received from, or provided to the data sources 3770, the data analytics system 952, and/or other processes implemented with the SaaS platform 3726 in a format other than the single standard data format to the single standard data format. For example, the integration platform 497 may convert a comma separated values (CSV) format, an extensible markup language (XML) or other text file format, or JavaScript® Object Notation (JSON) formatted files to the communication standard. The integration platform 497 may provide the data analytics system 952, and/or other processes implemented with the SaaS platform 3726 with access to the data sources 3770 by translating communications from processes connecting to the integration platform 497 into one or more communications standards recognized and/or utilized by the data sources 3770. The communications standards may include, for example, but not limited to, a Health Level Seven (HL7) standard provided by an HL7 standard integration 199b and/or a Substitutable Medical Applications and Reusable Technologies (SMART) on Fast Healthcare Interoperability Resources (FHIR) standard provided by a SMART on FHIR integration 199c. Optionally, the integration platform 497 may provide the billing system 3767 with access to the data sources 3770. The integration platform 497 may be configured to recognize a variety of communications standards adopted by users of the data sources 3770 such as, in some examples, HL7 version 2.x, HL7 version 3, Clinical Document Architecture (CDA), Continuity of Care Document (CCD), Fast Healthcare Interoperability Resources (FHIR), and/or file transfer protocol (FTP). As such, the integration platform 497 may be considered to be an aggregator that aggregates communications presented in a variety of forms, such as a variety of HL7 standard formats, into a single format (e.g., HL7 version 3, FHIR) or a set of formats (e.g., one version of the HL7 standard plus SMART on FHIR). In some implementations, the HL7 standard integration 199b is used for connecting to the data sources 3770 via a web application, while the SMART on FHIR integration 199c is used when accessing the data sources 3770 via a portal (e.g., using a browser or uniform resource identifier (URI)-based connection, such as a uniform resource locator (URL)-based connection).

In an implementation, a data analytics system (e.g., as provided by one or more data analytics system servers 952) may be communicatively coupled via multiple networked connections to a plurality of data sources 3770. The data sources 3770 may include patient data corresponding to a plurality of patients, payor data corresponding to a plurality of payers, provider data corresponding to a plurality of providers, and/or combinations thereof. These resources may be physically separated (e.g., geographically separated and/or resources associated with physically separated servers) and/or communicatively separated (e.g., associated with different business entities and/or accounts where there communicative couplings are unavailable between entities and/or accounts). For example, multiple medical records sources may be associated with different hospitals or medical provider systems, multiple sources of provider data may be associated with different medical service providers, the one or more sources of payor data may be associated with different insurance companies, etc. There may be instances of communications between some of the data sources 3770. For example, a medical provider may access patient data for their own patients without access to the patient data for patients associated with a different provider, a medical provider may access insurance payor data, each payor may access their own historic claims data without access to the historic claims data of another payor, etc.

In addition, the one or more data sources 3770 may be associated with one or more data resource access interface(s) 3715. A data resource access interface is configured to interact with a respective data resource. For example, each hospital, medical services provider, payor, insurance company, etc. may provide their own access interface such as an automated data entry system and/or a user terminal for data entry. The data resource access interface(s) 3715 (e.g., access software, hardware, firmware, and combinations thereof) may enable a particular data source to receive and store data and/or to provide access to previously stored data). In an implementation, the access interface(s) 3715 may enable automated computing systems and/or users, which may include providers, patients, and/or payers, to populate a data source and/or access or create medical records. At an access interface, an entity included in the data sources 3770 may utilize the data analytics services along with their own services and provide data that becomes part of the pool of data available to the data analytics service. For example, a medical billing service may provide a user terminal at which a biller can enter medical claims information for a patient and view output about past claims history from the data analytics service. The medical claims information entered by the biller then becomes part of the pool of information available to the data analytics service and informs future predictive intelligence generated by the data analytics service. The access interface(s) 3715 may enable data transfer between various data sources. For example, an access interface 3715 may enable a recorder of patient data to access data from or provide data to the medical record repository 3705. Although shown outside of the data sources 3770 for clarity of various implementations herein, the medical record repository 3705 may be included in the data sources 3770. As another example, an access interface 3715 may enable a medical claims recorder to access data from or provide data to a payor, provider, or claims and billing platform. Importantly, the data resource access interface(s) 3715 provide access to a respective data source. However, no single data resource access interface(s) 3715 provides access to all of the data sources 3770.

The physical processors described herein are physical processors (i.e., an integrated circuit configured to execute operations on a respective device as specified by software and/or firmware stored in a computer storage medium) operably coupled, respectively, to at least one memory device. The processors may be intelligent hardware devices (for example, but not limited to, a central processing unit (CPU), a graphics processing unit (GPU), a neural processing unit (NPU), one or more microprocessors, a controller or microcontroller, an application specific integrated circuit (ASIC), a digital signal processor (DSP), etc.) designed to perform the functions described herein and operable to carry out instructions on a respective device. Each of the processors may be one or more processors and may be implemented as a combination of hardware devices (e.g., a combination of DSP and a microprocessor, a plurality of microprocessors, one or more microprocessors in conjunction with a DSP core, or another such configuration). Each of the processors may include multiple separate physical entities that may be distributed in an associated computing device. Each of the processors is configured to execute processor-readable, processor-executable software code containing one or more instructions or code for controlling the processors to perform the functions as described herein. The processors may utilize various architectures including but not limited to a complex instruction set computer (CISC) processor, a reduced instruction set computer (RISC) processor, or a minimal instruction set computer (MISC). In various implementations, each processor may be a single-threaded or a multi-threaded processor. The processors may be, for example, but not limited to, an Intel® Itanium® or Itanium 2® processor(s), AMD® Opteron®, Athlon MP® processor(s), a Motorola® line of processor, or an ARM, Intel Pentium Mobile, Intel Core i5 Mobile, AMD A6 Series, AMD Phenom II Quad Core Mobile, or like devices.

The memories refer generally to a computer storage medium, including but not limited to RAM, ROM, FLASH, disc drives, fuse devices, and portable storage media, such as Universal Serial Bus (USB) flash drives, etc. Each of the memories may include, for example, random access memory (RAM), or another dynamic storage device(s) and may include read only memory (ROM) or another static storage device(s) such as programmable read only memory (PROM) chips for storing static information such as instructions for a coupled processor. Each memory may include USB flash drives that may store operating systems and other applications. The USB flash drives may include input/output components, such as a wireless transmitter and/or USB connector that can be inserted into a USB port of another computing device. Each memory may be long term and/or short term and is not to be limited to a particular type of memory or number of memories, or type of media upon which memory is stored. Each memory includes a non-transitory processor-readable storage medium (or media) that stores the processor-readable, processor-executable software code. Each memory may store information and instructions. For example, each memory may include flash memory and/or another storage media may be used, including removable or dedicated memory in a mobile or portable device. As another example, hard disks such as the Adaptec® family of SCSI drives, an optical disc, an array of disks such as RAID (e.g. the Adaptec family of RAID drives), or another mass storage device may be used. Each memory may include removable storage media such as, for example, external hard-drives, floppy drives, flash drives, zip drives, compact disc-read only memory (CD-ROM), compact disc-re-writable (CD-RW), or digital video disk-read only memory (DVD-ROM.

Communicatively coupled devices as described herein may transmit and/or receive information via a wired and/or wireless communicative coupling. The information may include information stored in at least one memory. The information may include, for example, but not be limited to, resuscitative treatment information, physiological information, patient information, rescuer and/or caregiver information, location information, rescue and/or medical treatment center information, etc. The communicative couplings may enable short-range and/or long-range wireless communication capabilities which may include communication via near field communication, ZIGBEE, Wi-Fi, BLUETOOTH, satellite(s), radio waves, a computer network (e.g., the Internet), a cellular network, a LAN, WAN, a mesh network, an ad hoc network, or another network. The communicative couplings may include, for example, an RS-232 port for use with a modem based dialup connection, a copper or fiber 10/100/1000 Ethernet port, or a BLUETOOTH or Wi-Fi interface.

Displays as described herein may provide a graphical user interface (GUI). A particular display may be, for example, but not be limited to, a touchscreen display, an AR display, a liquid crystal display (LCD), and/or a light emitting diode (LED) display. The touchscreen may be, for example, a pressure sensitive touchscreen or a capacitive touchscreen. The touchscreen may capture user input provided via touchscreen gestures and/or provided via exertions of pressure on a particular area of the screen. The displays may provide visual representations of data captured by and/or received at the medical device 132. The visual representations may include still images and/or video images (e.g., animated images).

The computing devices referred to herein may include one or more user input devices such as, for example, a keyboard, a mouse, joystick, trackball, or other pointing device, a microphone, a camera, etc. In an implementation, the user input devices may be configured to capture information, such as, for example, patient medical history (e.g., medical record information including age, gender, weight, body mass index, family history of heart disease, cardiac diagnosis, co-morbidity, medications, previous medical treatments, and/or other physiological information), physical examination results, patient identification, caregiver identification, healthcare facility information, etc.

The processor, memory, communication interfaces, input and/or output devices and other components described above are meant to exemplify some types of possibilities. In no way should the aforementioned examples limit the scope of the disclosure, as they are only exemplary embodiments of these components.

Various modifications and additions can be made to the exemplary embodiments discussed without departing from the scope of the present disclosure. For example, while the embodiments described above refer to particular features, the scope of the disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

It should be noted that the devices described herein can be used in medical settings other than EMS. For instance, some examples can be useful in hospital, clinic, military medical treatment, home, and other non-EMS settings. It should also be noted that EMS care can include both emergency care (e.g., car accident, cardiac arrest, overdose, etc.) and scheduled non-emergency care like a transport for dialysis, chemotherapy, physical therapy, and the like.

Having thus described several aspects of at least one example, it is to be appreciated that various alterations, modifications, and improvements will readily occur to those skilled in the art. For instance, examples disclosed herein can also be used in other contexts. Such alterations, modifications, and improvements are intended to be part of this disclosure and are intended to be within the scope of the examples discussed herein. Accordingly, the foregoing description and drawings are by way of example only.

1-106. (canceled)

107. An emergency medical services (EMS) charting system for documenting an EMS call, the EMS charting system comprising:
a first mobile device configured to
receive user input specifying first data to be stored in a first electronic patient care record (ePCR),
store the first data in the first ePCR in response to receipt of the user input, and
selectively forward the first data stored in the first ePCR to other mobile devices dispatched to the EMS call; and
a second mobile device configured to
receive other user input specifying second data to be stored in a second ePCR,
store the second data in the second ePCR in response to receipt of the other user input,
selectively forward the second data stored in the second ePCR to the other mobile devices dispatched to the EMS call,
receive the first data from the first mobile device,
display, via a user interface, the first data in a common screen with the second data, and
indicate that the first data was generated prior to arrival of the second mobile device at the EMS call.

108. (canceled)

109. The EMS charting system of claim 108, wherein:
the second mobile device is associated with a member of a second EMS team and configured to
receive data specifying that a first EMS team is assigned to the EMS call, the data comprising a reference code of a patient; and
receive the first data generated by the first EMS team prior to arrival of the second EMS team.

110. (canceled)

111. The EMS charting system of claim 109, wherein to receive the data specifying that the first EMS team is assigned to the EMS call comprises to acquire an image of a fiducial.

112. The EMS charting system of claim 109, wherein the second mobile device is configured to receive user input authorizing receipt of the first data prior to receipt of the first data.

113. The EMS charting system of claim 109, wherein the reference code is a driver's license number.

114. The EMS charting system of claim 109, wherein the first mobile device is configured to:
receive additional data generated after a patient is transferred to a healthcare provider other than the first EMS team and the second EMS team; and
display, within a chronology screen, additional data generated by the first EMS team, the second EMS team, and the healthcare provider, the additional data comprising an outcome of the EMS call.

115. (canceled)

116. The EMS charting system of claim 107, wherein the first mobile device is configured to:
display a medications screen comprising one or more medication control groups associated with one or more medications;
receive gesture input specifying medication information regarding the one or more medications; and
store, in response to reception of the gesture input, data specifying the medication information.

117. The EMS charting system of claim 116, wherein:
the one or more medication control groups comprise one or more name controls; and
the medication information comprises one or more names of the one or more medications.

118. The EMS charting system of claim 116, wherein:
the one or more medication control groups comprise one or more dose controls; and
the medication information comprises one or more doses of the one or more medications.

119. (canceled)

120. (canceled)

121. The EMS charting system of claim 116, wherein:
the one or more medication control groups comprise one or more frequency controls; and
the medication information comprises one or more frequencies of the one or more medications.

122. The EMS charting system of claim 107, wherein the first mobile device is configured to:
display a chronology screen comprising a plurality of time entry control groups associated with a plurality of timeline entries, each of the plurality of time entry control groups comprising at least one time entry control and at least one source control; and
display, via the at least one source control, an indication of a source of a corresponding timeline entry of the plurality of timeline entries.

123. The EMS charting system of claim 122, wherein the indication is of a medical device.

124. The EMS charting system of claim 123, wherein the medical device comprises one or more of a patient monitor, defibrillator, ventilator, or automated compression device.

125. The EMS charting system of claim 122, wherein the indication is of an EMS platform service.

126. The EMS charting system of claim 125, wherein the EMS platform service comprises one or more of a computer aided dispatch system, a navigation system, a billing system, a charting system, a case data store, a data analytics system, a hospital information system, or a medical data repository.

127. The EMS charting system of claim 122, wherein first mobile device is configured to provide, via the at least one time entry control, a visual representation of a timeline entry of the plurality of timeline entries.

128. The EMS charting system of claim 127, wherein the visual representation is of one or more of a call time, a dispatch time, a response time, an on-scene time, a time at which an EMS caregiver reached a patient, a time at which vital signs of the patient were measured, a time at which medication was administered to the patient, a time at which patient transport to a hospital initiated, a time at which the patient was admitted to the hospital, or a time at least the patient was discharged from the hospital.

129. The EMS charting system of claim 122, wherein:
each of the plurality of time entry control groups comprises at least one owner control; and
the first mobile device is configured to display, via the at least one owner control, an indication of an owner of a corresponding timeline entry of the plurality of timeline entries.

130. The EMS charting system of claim 129, wherein indication of the owner is an indication of one or more of a basic life support (BLS) team, an advanced life support (ALS) team, or a hospital department.

131. (canceled)

132. The EMS charting system of claim 107, wherein the first mobile device is configured to:
communicate additional data stored in the first ePCR to a data analytics system; and
receive further information regarding the additional data from the data analytics system.

133. The EMS charting system of claim 132, wherein:
the additional data is demographic data; and
the further information supplements the demographic data, corrects the demographics data, or both supplements and corrects the demographics data.

134-137. (canceled)

138. The EMS charting system of claim 107, wherein the first mobile device is configured to:
communicate additional data stored in the first ePCR to a computer aid dispatch (CAD) system; and
receive further information regarding the additional data from the CAD system.

139. The EMS charting system of claim 138, wherein the further information comprises dispatch information.

140. The EMS charting system of claim 139, wherein the dispatch information comprises a patient reference code.

141. The EMS charting system of claim 140, wherein the patient reference code is a driver's license number.

142-146. (canceled)
